SEQUENCE LISTING (208 sequences)

SEQ ID NO: 1 - protein, 205 aa, Clostridium cylindrosporum
  4-UREIDO-5-CARBOXYL-IMIDAZOLE-AMIDE HYDROLASE AND USE THEREOF
MARYGICVIDMLNDFIGESAPLRCPGGEEIVPNLQKLFKWARARMDAGHDDVHLIHIQEAHRKHDADFRVRPVHAIKGTHGSDFIKELYPEDRRCEYIIPKRRHSAFQHTDLDLYLREENLDTVVITGVWTNVCCRSTAADALANAYRVITLTDGNHSKTQAQHESGLVDLSIFTKLTTIDQFTADIDAGVDAWAGAGDQKNEVK

SEQ ID NO: 2 - protein, 200 aa, Sporanaerobacter acetigenes
  4-ureido-5-carboxyl-imidazole-amide hydrolase
MARHAILVIDMLNDFVGEKAPLRCPGGETIIPDLQRLFKWARGREGDDVQLVFIEEAHRENDADFRVRPVHAVKGTWGSDFIPELYPEKGEYIVQKRRHSGFAYTDLDLYLREENIDTVVMTGVWTNVCVRSTATDALARAYKVITLSDGCASKTEEMHEYGLNDLSIFSKIMTIDEYIDAWEKGLDPWEGGGDKENKVK

SEQ ID NO: 3 - protein, 194 aa, Thermosyntropha lipolytica
  4-ureido-5-carboxyl-imidazole-amide hydrolase
MAKDRHCIVIIDMLNDFIGPNAPLRCPDGEKIVPNLQKLVKFAHENGINVVFVQEAHRKDDADFKVRPVHAVKGTWGSDFIPELYPDEEKGDYIVQKRRHSAFAYTDLDLYLREEGIDTVVITGVWTNVCCRSTASDALYRCYKVVVISDCNASATQEMHEAGLRDMSLFAEVITLDEYMSKYGKQNGKFADAE

SEQ ID NO: 4 - protein, 197 aa, Aneurinibacillus migulanus
  4-ureido-5-carboxyl-imidazole-amide hydrolase
MLKGNKHAIVIIDMLNDFIGPKAPLRCEGGEKIVPNIQRLIEFAHENGIQVIHVQEWHRKDDADFRVRPVHAVRGTWGADFIPELRPEEDKGDYIVQKRRHSAFSYTDMDLFLREESIDTVVLTGVWTNVCVRSTGSDALYHGYNVVCISDGTSSQDEVMHESGLRDIGIFGQVFSTDEYIEQMKQKLAEKETNVSA

SEQ ID NO: 5 - protein, 200 aa, Alkaliphilus oremlandii
  4-ureido-5-carboxyl-imidazole-amide hydrolase
MPRHAILVIDMLNDFVGEKAPLRCPGGETIIPDLQKLFKWVRGRENDDVHLVHIQEAHRKNDADFRVRPVHAVDGTWGSDFIQELYPEEGEYIVKKRRHSGFAHTDLDLYLREENIDTVVVTGVWTNVCVRSTATDALANAYKVITLSDGCASKTEEMHEYGLNDLSIFTKVMTIDEYVAAWERGEDPWLGGGDRENKVE

SEQ ID NO: 6 - protein, 196 aa, Fictibacillus enclensis
  4-ureido-5-carboxyl-imidazole-amide hydrolase
MELRGKKHVVVVIDMLNDFIGEKAALRCENGEKIVPAIKELVDFCHEENIQVVFVQEAHRQNDRDFRVRPVHAIKGTWGSQFIPELQPDESKGDYVVQKRRHSAFSYTDFDLFCREEEIDTVVLTGVWTNVCVRSTGSDALYHGYNVICLSDSTASKDDSMHESGLRDIDLFGDIMTTKEYMEAARKKFSNNNQVV

SEQ ID NO: 7 - protein, 182 aa, Bacillus sp.
  4-ureido-5-carboxyl-imidazole-amide hydrolase
MLNDFIDEKGALCCKNGVKIVPKIRELVDFAHDNNIQVVHVQEAHRKNDRDFRVRPVHAVKGTWGSDFIPELKPEEEKGDYIVQKRRHSAFSFTDFDLFLREEEIDTVVLTGVWTNVCVRSTASDALYHGYNVICISDGTTSQDEDMHVSGLRDIDIFGDIFSTEEYKEAVKSKFSQDEQVV

SEQ ID NO: 8 - protein, 200 aa, Bacillus mesonae
  4-ureido-5-carboxyl-imidazole-amide hydrolase
MELRGNKHAVVIIDMLNDFIGEKAALRCENGEKIVPQIRDLIDFAHENNIQVIHVQEAHRKNDRDFRVRPVHAIKGTWGSEFIPELTPEEDKGDYVVQKRRHSAFSYTDFDLFCREEEIDTLVLTGVWTNVCVRSTASDGLYHGYNIICISDATASQDDDMHRSGLRDIDIFGDIMTTDEYKEAAKEKFSQSEQENEQAV

SEQ ID NO: 9 - protein, 242 aa, Bacillus oceanisediminis
  4-ureido-5-carboxyl-imidazole-amide hydrolase
MACGKRELKGDPAGALRRSTRGFEHLERKSTDKDNRANNKERGWVWMELRGNKHCVVIIDMLNDFIGEKAALRCENGEKIVPKIQELIEFAHENNIQVVHVQEAHRKNDRDFRVRPVHAVKGTWGSQFIPELQPDESKGDYVVQKRRHSAFSYTDFDLFLREEEIDTVVLTGVWTNVCVRSTASDALYHGYNVICISDGTASQDDDMHVSGLRDIDIFGDILTTEEYKEMAKKKFSQDNKVV

SEQ ID NO: 10 - protein, 196 aa, Bacillus firmus
  4-ureido-5-carboxyl-imidazole-amide hydrolase
MELRGKKHCVVIIDMLNDFIGEKAALRCENGEKIVPKIQELIQFAHEHNIQVVHVQEAHRKNDRDFRVRPVHAVKGTWGSEFIPELQPDETKGDYVVQKRRHSAFSYTDFDLFLREEEIDTVVLTGVWTNVCVRSTASDALYHGYNVICISDATASQDDDMHVSGLRDMDIFGDILSTESYKKLAKKKFSQDESVV

SEQ ID NO: 11 - protein, 200 aa, Marinisporobacter balticus
  4-ureido-5-carboxyl-imidazole-amide hydrolase
MARHAILVIDMLNDFIGEKAPLRCPGGDDIVPTLQELLKWVRAREDDDIHLVHIQEAHRKNDADFRVRPVHAVKGTWGSDFIKELYPEGEEYIVPKRRHSGFAHTDLDLYLREENIDTVVLTGVWTNVCVRSTASDALYNAYKVITLSDGCASKTEEMHAYGLNDLSIFTKVMTVDAYKKAWENGEDAWAGGGDAENKVK

SEQ ID NO: 12 - protein, 200 aa, Alkaliphilus peptidifermentans
  4-ureido-5-carboxyl-imidazole-amide hydrolase
MPRHAILVIDMLNDFVGVKAPLRCPGGETIIPDLQKLFKWVRSRKDDDIHLIHIQEAHRKNDADFRVRPVHAVKGTWGSDFIPELYPEGDEYIVPKRRHSAFAHTDLDLYLREENIDTVVVTGVWTNVCVRSSATDALANAYKVITLSDGCASKTEEMHLNGLKDLSIFTKIMTIDDYINAWEKGEDPWIGGGDAKNEVE

SEQ ID NO: 13 - protein, 182 aa, Bacillus firmus
  4-ureido-5-carboxyl-imidazole-amide hydrolase
MLNDFIGEKAALRCENGEKIVPKIQELIEFAHNHNIQVVHVQEAHRKNDRDFRVRPVHAVKGTWGSEFIPELQPDESKGDYVVQKRRHSAFSYTDFDLFLREEEIDTVVLTGVWTNVCVRSTASDALYHGYNVICVSDATASQDDDMHVSGLRDIDIFGDILSTEEYKELAKKKFSQDESVV

SEQ ID NO: 14 - protein, 192 aa, Thermoflavimicrobium dichotomicum
  4-ureido-5-carboxyl-imidazole-amide hydrolase
MKGEKHALVIIDMLNDFIGPKATLRCENGEKIVPNIRKLIDFCHEVGIQVIHVQEWHRPNDADFRVRPVHAVRGTWGAEFIPELQPEEEKGDYIVRKRRHSAFSYTDMDLFLREEKIDTVVLTGVWTNVCVRSTGSDALYHGYNVIAISDACASQTEEMHVSGLRDLGIFGEIFSTEEYIRMAREKFGAKAV

SEQ ID NO: 15 - protein, 182 aa, Bacillus oceanisediminis
  4-ureido-5-carboxyl-imidazole-amide hydrolase
MLNDFIGEKAALRCENGEKIVPKIQELIQFAHEHNIQVVHVQEAHRKNDRDFRVRPVHAVKGTWGSEFIPELQPDESKGDYVVQKRRHSAFSYTDFDLFLREEEIDTVVLTGVWTNVCVRSTASDALYHGYNVICISDATASQDDDMHVSGLRDIDIFGDILSTENYKMLAKKKFSQDESVV

SEQ ID NO: 16 - protein, 186 aa, Paenibacillus sp.
  4-ureido-5-carboxyl-imidazole-amide hydrolase
MGKHAIVVIDMLNDFIHPDGALTCPNGAGIVPALQDLIAFSHENEIQVIFVQEAHRKNDADFRVRPVHAIKGSWGSDFIAELTPDESKGDYVVQKRRHSAFSYTDMDLFLREEGIDTVAVTGVWTNVCVRSTASDAMYHTYKVVCISDCCASKDEEMHLSGLRDIGIFGEVVTLEQYKEKYAVVKG

SEQ ID NO: 17 - protein, 200 aa, Caloranaerobacter sp.
  4-ureido-5-carboxyl-imidazole-amide hydrolase
MPRHAILVIDMLNDFIGEKAPLRCPGGEKIVPNLQRLFEWVRNREDDDVVLVHIQEAHRKNDADFRVRPVHAVKGTWGSDFIKELYPQEDEYIVPKRRHSGFAYTDLDLYLREENIDTVVVTGVWTNVCVRSTASDALYHAYKVIALSDGCASKTEEMHEYGLKDMSLFAKVMTIDEYIDAWEKGIDPWLGGGDTENKVE

SEQ ID NO: 18 - protein, 197 aa, Virgibacillus profundi
  4-ureido-5-carboxyl-imidazole-amide hydrolase
MTVLKGKKHALVVIDMLNDFIGEKAALRCANGEKIVPKIREAIDFCHENDIQVINVQEAHRKNDADFRVRPVHAIKGTWGSQFIPELEPQEDKGDYVVQKRRHSAFSFTDFDLFLREEQIDTVVLTGVWTNVCVRSTASDALYQGYNVIALSDGTASKDDDMHRSGLRDMDLFGEIMTTDDYKEGAKKKFSQNRQTV

SEQ ID NO: 19 - protein, 191 aa, Carboxydothermus islandicus
  4-ureido-5-carboxyl-imidazole-amide hydrolase
MKGDRHCIVIIDMLNDFIGPNAPLRCPDGEKIVPNLQKLVEFAHENGINVVFVQEAHRKNDADFRVRPVHAVKGTWGSDFIPELRPDEEKGDYIVQKRRHSAFAYTDLDLYLREEKIDTVVVTGVWTNVCVRSTASDALYHAYNVIAISDCCASANEEMHQAGLRDMALFAEVMTLEEYMREWPNREGARK

SEQ ID NO: 20 - protein, 200 aa, Natronincola peptidivorans
  4-ureido-5-carboxyl-imidazole-amide hydrolase
MPRHAILVIDMLNDFVGEKAPLRCPGGETIIPDLQRLFKWVRNRKGDDVHLVHIQEAHRKHDADFRVRPVHAVKGTWGSDFIPALYPEGDEYIVPKRRHSGFAHTDLDLYLREEAIDTVVVTGVWTNVCVRSTATDALAHAYKVITLSDGCASKTEEMHQYGLNDLSIFTKVMTIDEYIEAWEKGEDPWVGGGDTENRVK

SEQ ID NO: 21 - protein, 203 aa, [Clostridium] ultunense Es
  4-ureido-5-carboxyl-imidazole-amide hydrolase
MTRHAILVIDMLNDFANEKGALYCPGAARITKNLQELLKWARDRKAEGKDDVQIVHIQEAHREDDADFRVRPRHAVRGTWGSDFIEELYPERGEYIVPKRRHSGFAYTDLDLYLREKGIDTVVVTGVWTNVCVRSTATDALARAYKVITLSDGCDSKTEEMHEYGLKDLSIFSKVMTIDEYIDAWQKGLDPWEGGGDPENKVE

SEQ ID NO: 22 - protein, 196 aa, Bacillus firmus
  4-ureido-5-carboxyl-imidazole-amide hydrolase
MELRGKKHCVVIIDMLNDFIGEKAALRCENGEKIVPKIQELIEFAHDHNIQVVHVQEAHRKNDRDFRVRPVHAIKGTWGSEFIPELQPDESKGDYVVQKRRHSAFSYTDFDLFLREEEIDTVVLTGVWTNVCVRSTASDALYHGYNVICVSDATASQDDDMHVSGLRDIDIFGDILSTEEYKELAKKKFSQDESVV

SEQ ID NO: 23 - protein, 186 aa, Paenibacillus typhae
  4-ureido-5-carboxyl-imidazole-amide hydrolase
MGKHAIVIIDMLNDFIHPDGALTCPNGAGIVPALQDLIAFSHENEIQVIFVQEAHRKNDADFRVRPVHAIKGSWGSDFIAELTPDESKGDYVVQKRRHSAFSYTDMDLFLREEGIDTVAVTGVWTNVCVRSTASDAMYHTYKVVCISDCCASKDEEMHQSGLRDIGIFGEVVTLEQYKEKYAVVKG

SEQ ID NO: 24 - protein, 186 aa, Paenibacillus sp.
  4-ureido-5-carboxyl-imidazole-amide hydrolase
MGKHAIVVIDMLNDFIHPDGALTCPNGAGIVPALQDLIAFSHENEIQVIFVQEAHRKNDADFRVRPVHAIKGSWGSDFIAELTPDESKGDYVVQKRRHSAFSYTDMDLFLREEGIDTVAVTGVWTNVCVRSTASDAMYHTYKVVCISDCCASKDEEMHRSGLRDIGIFGEVVTLEQYKEKYAVVKG

SEQ ID NO: 25 - protein, 195 aa, Natribacillus halophilus
  4-ureido-5-carboxyl-imidazole-amide hydrolase
MELKGNKHAVVVIDMLNDFISEGGALRVENNEKIVPKIREMIDFSHENDIQVVFIQEAHRKNDADFKVRPVHAIKGTWGAEFIPELQPDEDKGDYIVQKRRHSAFAHTDFDLFLREEGIDTVVLTGNWTNVCVRSTASDALYYTYNVVCLSDATASKTDEMHKAGLVDMGLFGQVMTTDEYKKGVKQKFSQKQSN

SEQ ID NO: 26 - protein, 203 aa, Caloramator australicus
  4-ureido-5-carboxyl-imidazole-amide hydrolase
MPRHAILVIDMLNDFVGEKATLRCPGADLIVPNLQKLFKWVRERRANGKDEVQIVHIQEAHRKNDADFRVRPVHAVKGTWGSDFIDELKPEGDEYIVQKRRHSGFAYTDLDLYLREENIDTVVLTGVWTNVCVRSTASDAIHRAYKVIVLSDGTQSKTQEMHESGLRDLSLFSKIMTIDEYIDAWEKGIDPWTGAGDRENKVE

SEQ ID NO: 27 - protein, 196 aa, Bacillus sp.
  4-ureido-5-carboxyl-imidazole-amide hydrolase
MELRGKKHCVVIIDMLNDFIGEKAALRCENGEKIVPKIQELIEFAHDHNIQVVHVQEAHRKNDRDFRVRPVHAIKGTWGSEFIPELQPDESKGDYVVQKRRHSAFSYTDFDLFLREEEIDTVVLTGVWTNVCVRSTASDALYHGYNVICVSDATASQDDDMHVSGLRDIDIFGDILSTEEYKELAKTKFSQDESVV

SEQ ID NO: 28 - protein, 186 aa, Paenibacillus sp.
  4-ureido-5-carboxyl-imidazole-amide hydrolase
MAKHAVVIIDMLNDFIHPDGALTCPNGAGIVPAIQELIEFSHENDIQVIFVQEAHRQNDADFRVRPVHAIKGTWGSDFIAELTPQPDKGDYIVQKRRHSAFSYTDMDLFLREEGIDTVVVTGVWTNVCVRSTASDAMYHTYKVKCISDCCASKDEEMHISGLRDIGIFGEVLTLAEYKEVNAVVKG

SEQ ID NO: 29 - protein, 191 aa, Carboxydothermus hydrogenoformans
  4-ureido-5-carboxyl-imidazole-amide hydrolase
MKGDRHCIVIIDMLNDFIGPNAPLRCPDGEKIVPNLQKLVEFAHENGINVVFVQEAHRKNDADFRVRPVHAVKGTWGSDFIPELRPDEEKGDYIVQKRRHSAFAYTDLDLYLREEKIDTVVVTGVWTNVCVRSTASDALYHAYNVIAISDCCASANEEMHQAGLRDMALFAEVMTLEEYMKEWPNREGAKK

SEQ ID NO: 30 - protein, 197 aa, Bacillus sp.
  4-ureido-5-carboxyl-imidazole-amide hydrolase
MAKLKGEKHALVIIDMLNDFIGEKAVLRCENGEKIVSKIREAIDFCHDNGIQVVHVQEAHRKNDADFRVRPVHAIKGTWGSQFIDELQPEEDKGDYIVQKRRHSAFSYTDFDLFLREEKIDTVVLTGVWTNVCVRSTASDALYHGYNVIALSDATASQDEDMHRGGLRDIGIFGEVMTTDEYMDAAKEKFSQDAQTV

SEQ ID NO: 31 - protein, 187 aa, Bacillus sp.
  4-ureido-5-carboxyl-imidazole-amide hydrolase
MVIIDMLNDFIGEKAALRCENGEKIVPKIQELIQFAHEHNIQVVHVQEAHRKNDRDFRVRPVHAVKGTWGSEFIPELQPDESKGDYVVQKRRHSAFSYTDFDLFLREEEIDTVVLTGVWTNVCVRSTASDALYHGYNVICISDATASQDDDMHVSGLRDIDIFGDILSTENYKMLAKKKFSQDESVV

SEQ ID NO: 32 - protein, 203 aa, Paraclostridium benzoelyticum
  4-ureido-5-carboxyl-imidazole-amide hydrolase
MKRHAILVIDMLNDFVGEKAPLRCPGGEKIIPDLQKLFAWVRERNARGLDDVQLVHIQEAHRKNDADFRVRPVHAVKGTWGSDFIKELYPQGEEYIVPKRRHSGFAHTDLDLYLREENIDTVVVTGVWTNVCVRSTASDALYNAYKVITLSDGVHSKNDEMHEAGLRDLSIFTKVMTIDEYIDAWEKNLDPWVGGGDTENKVE

SEQ ID NO: 33 - protein, 207 aa, Gottschalkia acidurici
  4-ureido-5-carboxyl-imidazole-amide hydrolase
MSNQHRYGIVVIDMLNDFIGEKAPLRCPGGEEIVPNLQKLFKWVRERNAAGKDDVQLIHIQEAHRKNDADFRVRPVHAIKGTWGSDFIKELYPEGEEYAIPKRRHSSFWHTDLDLYLREENLDTVVVTGVWTNVCVRSTAADALALAYRVITLRDGVHSKDEAQHQNGLVDLSIFTKVIDIDEFTSAIDAGNDPWAGAGDQQNKVQG

SEQ ID NO: 34 - protein, 196 aa, Bacillus notoginsengisoli
  4-ureido-5-carboxyl-imidazole-amide hydrolase
MELRGNKHAIVIIDMLNDFIGEKAALRCEAGEKIVPKIRELIDFAHVNNIQVIHVQEAHRKNDRDFRVRPVHAIKGTWGSQFIPELTPEEDKGDYVVQKRRHSAFSYTDFDLFCREEEIDTLVLTGVWTNVCVRSTASDGLYHGYNIICVSDATASQDEDMHRSGLRDIDIFGDIMTTDEYKEAAKEKFSHNEQAV

SEQ ID NO: 35 - protein, 203 aa, Romboutsia lituseburensis
  4-ureido-5-carboxyl-imidazole-amide hydrolase
MPRHGIVVIDMLNDFVGEKAPLRCPGGEKIIPNLQKLFAWVRERNARGLDDIQLIHIQEAHRKNDADFRVRPVHAVKGTWGSDFIKELYPQGEEYTIPKRRHSAFAHTDLDLYLREENLDTVVVTGVWTNVCVRSTASDALYNAYKVITLSDGVHSKNDEMHEAGLRDLSIFTKIMTIDDYIDAWEKGIDPWQGGGDKENKVE

SEQ ID NO: 36 - protein, 191 aa, Carboxydothermus pertinax
  4-ureido-5-carboxyl-imidazole-amide hydrolase
MKGDRHCIVIIDMLNDFIGPNAPLRCPDGEKIVPNLQKLVEFAHEKGINVVFVQEAHRKNDADFRVRPVHAIKGTWGSDFIPELRPDEEKGDYIVQKRRHSAFAYTDLDLYLREEKIDTVVVTGVWTNVCVRSTASDALYHAYNVIAISDCCASKDEEMHQAGLRDMALFAEVMTLEEYMKEWPNREGAKR

SEQ ID NO: 37 - protein, 203 aa, Acidaminobacter hydrogenoformans
  4-ureido-5-carboxyl-imidazole-amide hydrolase
MPRHGIVVIDMLNDFIGEKAMLRCPGGDDIIAPLQKLFAWVRERNAQGNDDIQLIHIQEAHRKNDADFRVRPVHAIAGTWGSDFIPELYPEGEEYKIPKRRHSAFQHTDLDLYLKEENLDTVVVTGVWTNVCVRSTAADALANAYKVITLSDCVASKTEEMHEHGLVDLSIFTKVMTLDEYMAAWEKGEDPWLGGGDAENKVE

SEQ ID NO: 38 - protein, 203 aa, Soehngenia saccharolytica
  4-ureido-5-carboxyl-imidazole-amide hydrolase
MTKHAILVIDMLNDFADPNGVLYCPNSRKIIPTMQKLFDWVRERNRNGNDDVQLIFIQEAHRKNDADFKVRPVHAVKGTWGSDFIDELRPLDNEYIVQKRRHSAFAHTDLDLYCREEKIDTIVVTGTWTNVCVRSSASDALYYAYKVITLSDAVQSKTEEMHLSGLRDLSIFTKVMTVDEYIEAWDKGVDPWEGGGDKENKVE

SEQ ID NO: 39 - protein, 200 aa, Tindallia californiensis
  4-ureido-5-carboxyl-imidazole-amide hydrolase
MPRHAILVIDMLNDFVGEKAPLRCPGGDDIIEPLQKLFEWVRNRQDDDVHLVHIQEAHRKHDADFRVRPVHAVDGTWGSDFIPALYPEGDEYIVKKRRHSGFAHTDLDLYLKEENIDTVVVTGVWTNVCVRSTATDALANAYKVITLSDGCASKTDAMHEHGLVDLSIFTKVMSIDEYIDAWNKGEDPWIGGGDAENKVE

SEQ ID NO: 40 - protein, 196 aa, Bacillus sp.
  4-ureido-5-carboxyl-imidazole-amide hydrolase
MELRGKKHVVVVIDMLNDFIGEKAALRCENGEKIVPAIKELVDFCHEESIQVVFVQEAHRKNDRDFRVRPVHAIKGTWGSQFIPELQPDESKGDYIVQKRRHSAFSYTDFDLFCREEEIDTVVLTGVWTNVCVRSTGSDALYHGYNVICLSDSTASKDDSMHESGLRDIDLFGDIMSTKEYMEAARKKFSKNSQVV

SEQ ID NO: 41 - protein, 193 aa, Sulfobacillus benefaciens
  4-ureido-5-carboxyl-imidazole-amide hydrolase
MNNRALLVIDMINDLIKNPSNALYCPQTTQTVENIATLIDFAHKNQWKVVFVQDAHRIGDKDFTIRPVHAIRGTWGAELIDELAGLKQKSDYEVFKRRHSGFSYTDLDLYLREEHIEEVVLTGVWTNVAVRNTASDAFYQFYRVTVVTDCCQSQTEEMHESGLHDLSMFARLTLLQNVTSTDAFQYQLDDDTL

SEQ ID NO: 42 - protein, 182 aa, Bacillus solani; identical to SEQ ID NO: 7
  4-ureido-5-carboxyl-imidazole-amide hydrolase

SEQ ID NO: 43 - protein, 203 aa, Clostridium sp.
  4-ureido-5-carboxyl-imidazole-amide hydrolase
MKRHAILVIDMLNDFVGEKAPLRCPGGEKIIPDLQKLFAWVRERNARGLDDVQLVHIQEAHRKNDADFRVRPVHAVKGTWGSDFIKELYPQGEEYIVPKRRHSGFAHTDLDLYLREENIDTVVVTGVWTNVCVRSTASDALYNAYKVITLSDGVHSKNDEMHEAGLRDLSIFTKVMTIDEYIDAWEKDLDPWVGGGDTENKVE

SEQ ID NO: 44 - protein, 186 aa, Paenibacillus sp.
  4-ureido-5-carboxyl-imidazole-amide hydrolase
MAKHAVVIIDMLNDFIHPDGALTCPNGTGIVPAIQELIEFSHENDIQVIFVQEAHRQNDADFRVRPVHAIKGTWGSDFITELTPQPDKGDYIVQKRRHSAFSYTDMDLFLREEGIDTVVVTGVWTNVCVRSTASDAMYHTYKVKCISDCCASKDEEMHVSGLRDIGIFGEVLTLAEYKEVNAVVKG

SEQ ID NO: 45 - protein, 200 aa, Caloramator mitchellensis
  4-ureido-5-carboxyl-imidazole-amide hydrolase
MPRHAILVIDMLNDFVGEKATLRCPGADLIVPNLQRLFKWVRERGKDDIQLVHIQEAHRKNDADFRVRPRHAVKGTWGSEFIDELKPEGEEYVVPKRRHSGFAYTDLDLYLREENIDTVVLTGVWTNVCVRSTASDALHHAYKVIVLSDGTQSKTQEMHESGLRDLSLFSKIMTIDEYIDAWEKGIDPWTGAGDTQNKVE

SEQ ID NO: 46 - protein, 182 aa, Sporosarcina globispora
  4-ureido-5-carboxyl-imidazole-amide hydrolase
MLNDFIGEKAALRCENGEKIVPKIQELIQFAHDHNIQVVHVQEAHRKNDRDFRVRPVHAVKGTWGSEFIPELQPDESKGDYVVQKRRHSAFSYTDFDLFLREEEIDTVVLTGVWTNVCVRSTASDALYHGYNVICISDATASQDDDMHVSGLRDIDIFGDILSTEKYKELAKKKFSQDESVV

SEQ ID NO: 47 - protein, 203 aa, Paraclostridium bifermentans; identical to SEQ ID NO: 43
  4-ureido-5-carboxyl-imidazole-amide hydrolase

SEQ ID NO: 48 - protein, 203 aa, Proteiniborus sp.
  4-ureido-5-carboxyl-imidazole-amide hydrolase
MAKHAIVVIDMLNDFIGEKAPLRCPGGEEIVPNLQKLFKWVRERNAQGKDDVQLVHVQEAHRENDADFRVRPVHAVKGTWGSDFIKELYPEKGEYIIQKRRHSAFWHTDLDLFLREESIDTVVVTGVWTNVCVRSTAADALANAYKVITLSDGVHSKTEEQHQHGLVDLSIFTKVMTIDEYIDAFEKGLDPWAGAGDPENKVK

SEQ ID NO: 49 - protein, 200 aa, Maledivibacter halophilus
  4-ureido-5-carboxyl-imidazole-amide hydrolase
MPRHAIVVIDMLNDFIGEKAPLRCPGGDDIVPNLQRLFKWVRGRKDDDIHLVHIQEAHRKNDADFRVRPVHAVKGTWGSDFIKDLYPEGDEYTIPKRRHSAFAHTDLDLYLREENIDTVVVTGVWTNVCVRSTASDALYNAYKVITLSDACASKTEEMHQYGLNDLSIFTKVMTIDEYIDAWEKGEDPWLGGGDQENKVE

SEQ ID NO: 50 - protein, 203 aa, Tepidimicrobium xylanilyticum
  4-ureido-5-carboxyl-imidazole-amide hydrolase
MPRHGILIIDMLNDFAHPDGVLYCPTNRTIIPTLQKLMNWVRERNAQGKDDIQLIHIQEAHRENDADFKVRPVHAVKGTWGSEFIDELKPEKGEYIVQKRRHSAFAHTDLDLYLREEKIDTVVVTGTWTNVCVRSSASDALYYAYKVITLSDACASKTEEMHQAGLKDLSIFSKVMTVDEYIDAWEKGLDPWEGGGDPENKVE

SEQ ID NO: 51 - protein, 203 aa, Tissierella sp.
  4-ureido-5-carboxyl-imidazole-amide hydrolase
MPRHAILIIDMLNDFANPKGALFCEGTAIITPKLQEIFKWVRERNAEGKDDVQLVHIQEAHRKNDADFKVRPVHAVDGTWGSDFVTELKPEGDEYIVKKRRHSGFAYTDLDLYLREENIDTVVVTGTWTNVCVRSTATDALARAYKVISLTDAIHSKTQEMHENGLRDLSIFTKLMTFDEYKEAWEKGLDPWTGGGDPENKVK

SEQ ID NO: 52 - protein, 203 aa, Paraclostridium bifermentans
  4-ureido-5-carboxyl-imidazole-amide hydrolase
MKRHAILVIDMLNDFVGEKAPLRCPGGEKIIPDLQKLFAWVRERNARGLDDVQLVHIQEAHRKNDADFRVRPVHAVKGTWGSDFIKELYPQGEEYIVPKRRHSGFAHTDLDLYLREENIDTVVVTGVWTNVCVRSTASDALYNAYKVITLSDGVHSKNDEMHEAGLRDLSIFTKVMSIDDYIDAWEKDLDPWVGGGDTENKVE

SEQ ID NO: 53 - protein, 208 aa, Andreesenia angusta
  4-ureido-5-carboxyl-imidazole-amide hydrolase
MSNQHRYGIIVIDMLNDFIGEKAPLRCPGGEEIVPNLQKLFKWVRERNAGGKDDVQLIHIQEAHRKNDADFRVRPVHAIKGSWGSDFIKELYPEGEEYTIPKRRHSSFWHTDLDLYLREESLDTVVVTGVWTNVCVRSTAADALALAYRVITLSDGVHSKDQAQHEHGLVDLSIFTKVMTIDEFTSAIDAGIDAWAGAGDQENKVGEK

SEQ ID NO: 54 - protein, 203 aa, Tissierella praeacuta
  4-ureido-5-carboxyl-imidazole-amide hydrolase
MSRHAILVIDMLNDFANPEGALFCEGTAIITPKLQEIFKWVRERNAKGNDDVQLVHIQEAHRKNDADFKVRPVHAVDGTWGSDFVPELKPEGDEYIVKKRRHSGFAYTDLDLYLREENIDTVVVTGTWTNVCVRSTATDALARAYRVISLTDAIHAKNQEMHEQGLRDLSIFTKLMTFDEYKEAWEKGIDPWEGGGDPENKVK

SEQ ID NO: 55 - protein, 186 aa, Ammoniphilus sp.
  4-ureido-5-carboxyl-imidazole-amide hydrolase
MGKKALIVIDVINDFVKNSEGPLFCPSGKMVTRKIKESVAYFHETQQQVIYVHDSHRKNDAEFLLRPLHAISGSWGAELADELKADQEEEDYHILKRRHSAFSYTDLDLFLREEKIEEVVLVGGMTHVSIRSTASDAFYQAYQVTVLSDCCFSQSEELHQSGLQDIGMFATICTFHEFVGMTETER

SEQ ID NO: 56 - protein, 195 aa, Bacillus sp.
  4-ureido-5-carboxyl-imidazole-amide hydrolase
MELRGKKHAVVVIDMLNDFIGEKATLRCENGEKIVPEIRRLVDFAHDHNIQVIHVQEAHRQNDRDFRVRPVHAIKGTWGSEFIPELQPEEDKGDYVVQKRRHSAFSYTDFDLFCREEEIDTLVLTGVWTNVCVRSTASDGLYHGYNVICISDATASKDDDMHVSGLRDIDIFGDIMTVDQYTSLVKEKLSQNQKV

SEQ ID NO: 57 - protein, 193 aa, Marininema halotolerans
  4-ureido-5-carboxyl-imidazole-amide hydrolase
MKGNKHALVIIDMLNDFIGPEAALRCENGEKIVPHIKEAVDFAHTQGIQVVFVQEAHRKNDADFRVRPVHAIKGTWGSQFIEELQPDEAQGDYIVQKRRHSAFSYTDFDLFLREEGVDTVVLTGVWTNVCVRSTASDALYHGYNIICLSDSTASQDDDMHRSGLRDIALFGEVMSTEEYIALAKEKFPIERAI

SEQ ID NO: 58 - protein, 193 aa, Marininema mesophilum
  4-ureido-5-carboxyl-imidazole-amide hydrolase
MKGNKHALVIIDMLNDFIGPQAALRCENGEKIVPNIKETVNFAHAQGIQVVFVQETHRKNDADFRVRPVHAIKGTWGSQFIEELQPDESQGDYIVQKRRHSAFSYTDFDLFLREEAIDTVVLTGVWTNVCVRSTASDALYHGYNIICLSDSTASQDDDMHRSGLRDIGLFGEVMTTEDYITRAKEKFPVESAI

SEQ ID NO: 59 - protein, 204 aa, Unknown
  Clostridiaceae bacterium 4-ureido-5-carboxyl- imidazole-amide hydrolase
MGKHCILVIDMLNDFIGENAALRCPDGEKIVPALRKLMDYGRTRPEDIHIVHIQEAHRKHDADFRVRPVHAVAGTWGSRIIDELKPVGDEYVIPKRRHSGFMYTDLDLYLREENIDTVVVTGVWTNVCVRSTASDASYRAYKVIAISDCCASKDEEMHRAGLRDMAIFAKVMTVDEYIKAWEEGEDPWKDAGQPNNKEKGRCCS

SEQ ID NO: 60 - protein, 196 aa, Bacillus oceanisediminis
  4-ureido-5-carboxyl-imidazole-amide hydrolase
MELRGNRHCVVIIDMLNDFIGEKAALRCENGEKIVPKIQELIEFAHENNIQVVHVQEAHRKNDRDFRVRPVHAVKGTWGSQFIPELQPDESKGDYVVQKRRHSAFSYTDFDLFLREEEIDTVVLTGVWTNVCVRSTASDALYHGYNVICISDGTASQDDDMHVSGLRDIDIFGDILTTEEYKEMAKKKFSQDNTVV

SEQ ID NO: 61 - protein, 186 aa, Unknown
  compost metagenome 4-ureido-5-carboxyl- imidazole-amide hydrolase
MGKHAIVVIDMLNDFIHPDGALTCPNGAGIVPALQDLIEFSHENEIQVIFVQEAHRKNDADFRVRPVHAIKGSWGSDFIAELTPDESKGDYVVQKRRHSAFSYTDMDLFLREEGIDTVAVTGVWTNVCVRSTASDAMYHTYKVVCISDCCASKDEEMHQSGLRDIGIFGEVVTLAQYKEKYAVVKG

SEQ ID NO: 62 - protein, 200 aa, Thermotalea metallivorans
  4-ureido-5-carboxyl-imidazole-amide hydrolase
MARHAILVIDMLNDFIGEKAPLRCPGGDAIIPNLQKLFGWVRSRRDDNVQLVHIQEAHRKNDADFRVRPVHAVKGTWGSDFIPELYPQGDEYIVQKRRHSGFAYTDLDLYLREENIDTVVVTGVWTNVCVRSTASDALYHAYKVITLSDGCASKTEEMHEYGLKDLSIFTKVMTIDEYIEAWEKGEDPWIGGGDRENRVE

SEQ ID NO: 63 - protein, 203 aa, Caloramator fervidus
  4-ureido-5-carboxyl-imidazole-amide hydrolase
MPRHAILVIDMLNDFVGEKATLRCPGADLIVPNLQRLFKWVRERRASGKDDVQLVHIQEAHRKNDADFRVRPVHAVKGTWGSDFIEELKPEGDEYIVQKRRHSGFAYTDLDLYLREENIDTVVLTGVWTNVCVRSTASDALHHAYKVIVLSDGTQSKTQEMHESGLRDLSLFAKIMTIDEYIEAWENGIDPWIGAGDRENKVE

SEQ ID NO: 64 - protein, 193 aa, Paludifilum halophilum
  4-ureido-5-carboxyl-imidazole-amide hydrolase
MTGNKHALVIIDMLNDFIGPKAALRCENGEKIVANIRKAVDFAYNHDIQVVYIQEAHRKNDADFRVRPVHAIKGTWGSDFIEELRPDESKGDYVVQKRRHSAFSYTDFDLFLREEAIDTVVLTGVWTNVCVRSTASDALYHGYNIICLSDGTASKDDDMHRSGLRDIGLFGEVMTVDQYIEEAKKKFPAGSTV

SEQ ID NO: 65 - protein, 197 aa, Bacillus fortis
  4-ureido-5-carboxyl-imidazole-amide hydrolase
MTQLKGEKHALVIIDMLNDFIGEKAALRCENGEKIVPKIREAIDFCHENGIQVVHVQEAHRKNDADFRVRPVHAIKGTWGSEFIEELQPEEDKGDYIVQKRRHSAFSYTDFDLFLREEKIDTVVLTGVWTNVCVRSTASDALYHGYNVIALSDATASQDDDMHRGGLRDIGIFGEVMTTEEYMDAAKKKFSQDAQTV

SEQ ID NO: 66 - protein, 196 aa, Paenibacillus sp.; identical to SEQ ID NO: 22
  4-ureido-5-carboxyl-imidazole-amide hydrolase

SEQ ID NO: 67 - protein, 186 aa, Unknown; identical to SEQ ID NO: 16
  compost metagenome 4-ureido-5-carboxyl- imidazole-amide hydrolase

SEQ ID NO: 68 - protein, 186 aa, Thermosyntropha lipolytica
  4-ureido-5-carboxyl-imidazole-amide hydrolase
MPERHCITIIDMLNDFIGPKAPLRCPGGEAIVPNLQRLVKWAHEHENVRVVFVQEAHRKNDADFKVRPVHAIKGTWGSDFIPELRPDEEKGDYIVQKRRHSAFAYTDLDLYLREEGCDTVVVTGVWTNVCCRSTASDALYRAYKVIVISDCNASATEEMHQSGLRDMALFAEVITLDEYMKKYDYL

SEQ ID NO: 69 - protein, 196 aa, Fictibacillus sp.; identical to SEQ ID NO: 6
  4-ureido-5-carboxyl-imidazole-amide hydrolase

SEQ ID NO: 70 - protein, 197 aa, Bacillus terrae
  4-ureido-5-carboxyl-imidazole-amide hydrolase
MTKLKGEKHALVIIDMLNDFIGEKAALRCENGEKIVPKIREAIDFCHNYGIQVVHVQEAHRKNDADFRVRPVHAIKGTWGSEFIEELQPEEDKGDYVVQKRRHSAFSYTDFDLFLREEKVDTVVLTGVWTNVCVRSTASDALYHGYNVIALSDSTASQDEDMHRGGLRDIGIFGEVMTTDEYMDAAKKKFSQNAQTV

SEQ ID NO: 71 - protein, 196 aa, Bacillus firmus
  4-ureido-5-carboxyl-imidazole-amide hydrolase
MELRGKKHCVVIIDMLNDFIGEKAALRCENGEKIVPKIQELIEFAHNHNIQVVHVQEAHRKNDRDFRVRPVHAVKGTWGSEFIPELQPDESKGDYVVQKRRHSAFSYTDFDLFLREEEIDTVVLTGVWTNVCVRSTASDALYHGYNVICVSDATASQDDDMHVSGLRDIDIFGDILSTEEYKELAKKKFSQDESVV

SEQ ID NO: 72 - protein, 182 aa, Bacillus oceanisediminis; identical to SEQ ID NO: 15
  4-ureido-5-carboxyl-imidazole-amide hydrolase

SEQ ID NO: 73 - protein, 204 aa, Tissierella creatinini
  4-ureido-5-carboxyl-imidazole-amide hydrolase
MTRHAIVVIDMLNDFANPKGALYCEGAGRITKNLQALFKWVRARNAEGKNDIQLVHIQEAHRKNDADFRVRPVHAVDGTWGSDFIQELYPEGDEYRIKKRRHSSFAHTDLDLYLREENIDTVVVTGTWTNVCVRATATDALAKGYKVISLTDGIDSKTPEMHESGLRDLSIFTKLMTIAEYQEAWEKGLDPWEGGAGDPENKVK

SEQ ID NO: 74 - protein, 197 aa, Virgibacillus indicus
  4-ureido-5-carboxyl-imidazole-amide hydrolase
MAVLKGKKHALVIIDMLNDFIGEKAALRCENGETIVPKIREAIDFCHENDIQVIHVQEAHRKNDADFRVRPVHAIKGTWGSQFIPELEPEEDKGDYVVQKRRHSAFSFTDFDLFLREEQIDTVVLTGVWTNVCVRSTASDALYHGYNVIALSDATASKDDDMHRSGLRDMDLFGEIMTTDEYKEGAKKKFSQNRQTV

SEQ ID NO: 75 - protein, 196 aa, Bacillus praedii
  4-ureido-5-carboxyl-imidazole-amide hydrolase
MELKGNKHCIVVIDMLNDFIDEKGALCCKNGVKIVPKISELVDFAHENQIQVVHVQEAHRKNDRDFRVRPVHAVKGTWGSEFIPELQPNEEKGDYVVQKRRHSAFSFTDFDLFLREEEIDTVVLTGVWTNVCVRSTASDALYHGYNVICISDGTTSQDEDMHVSGLRDIDIFGDIFTTEEYKEAVKSKFSHDEKVV

SEQ ID NO: 76 - protein, 196 aa, Fictibacillus solisalsi
  4-ureido-5-carboxyl-imidazole-amide hydrolase
MELRGKKHVVVVIDMLNDFIGEKAALRCENGEKIVPAIKELVDFCHEENIQVVFVQEAHRKNDRDFRVRPVHAIKGTWGSQFIPELQPDESKGDYVVQKRRHSAFSYTDFDLFCREEEIDTVVLTGVWTNVCVRSTGSDALYHGYNVICLSDSTASKDDSMHESGLRDIDLFGDIMTTKEYMEAARKKFSNNNQVV

SEQ ID NO: 77 - protein, 203 aa, Acinetobacter sp.; identical to SEQ ID NO: 43
  4-ureido-5-carboxyl-imidazole-amide hydrolase

SEQ ID NO: 78 - protein, 186 aa, Paenibacillus sp.
  4-ureido-5-carboxyl-imidazole-amide hydrolase
MAKHAVVIIDMLNDFIHPDGALTCPNGAGIVPAIQELIEFSHENDIQVIFVQEAHRQNDADFRVRPVHAIKGTWGSDFITELTPQPDKGDYIVQKRRHSAFSYTDMDLFLREEGIDTVVVTGVWTNVCVRSTASDAMYHTYKVKCISDCCASKDEEMHISGLRDIGIFGEVLTLAEYKEVNAVVKG

SEQ ID NO: 79 - protein, 200 aa, Anaerovirgula multivorans
  4-ureido-5-carboxyl-imidazole-amide hydrolase
MSRHAVLVIDMLNDFVGERASLRCPGGETIIPDLQKLFKWIRSRKDDDIHLVHIQEAHRKNDADFRVRPAHAVKGSWGSDFIPELYPEGDEYIVPKRRHSGFAHTDLDLYLREEAIDTVVVTGVWTNVCVRSTATDALARAYKVITLSDGCASKTEEMHQYGLNDLSIFTKVMTIDEYIKAWKNSEDPWIGGGDTQNKVK

SEQ ID NO: 80 - protein, 203 aa, Tissierella praeacuta; identical to SEQ ID NO: 54
  4-ureido-5-carboxyl-imidazole-amide hydrolase

SEQ ID NO: 81 - protein, 200 aa, Alkaliphilus sp.
  4-ureido-5-carboxyl-imidazole-amide hydrolase
MSKYAILVIDMLNDFVGEKAPLRCPGGETITPDLQKLFKWVREREGDEVQLLHIQEAHRKNDADFRVRPVHAVKGTWGSDFIKELYPEEDEYIVQKRRHSGFAYTDLDLYLREENINTVVVTGVWTNVCVRSTASDALYSAYKVIALSDGCASKTEEMHEYGLNDLSLFAKILTIDEFTNAIDSGIDPWIGGGDTQNKVE

SEQ ID NO: 82 - protein, 186 aa, Paenibacillus donghaensis
  4-ureido-5-carboxyl-imidazole-amide hydrolase
MSKHAIVVIDMLNDFISPEGALTCPNGEKIVPALQELIEFSHENDIQVIFVQEAHRKNDADFRVRPIHAIKGTWGSEFIPELTPQPEQGDYVVQKRRHSAFSYTDMDLFLREEGIDTVVVTGVWTNVCVRSTASDAMYHTYKVMCISDCCASKDEEMHVSGLRDIGIFGEVLTLEEYKAKNTVVRG

SEQ ID NO: 83 - protein, 194 aa, Ammoniphilus sp.
  4-ureido-5-carboxyl-imidazole-amide hydrolase
MKGNKHALVIIDMLNDFIGPKASLRCEAGEEIVPNIRKLIDFAHDHGIQVIHVQEWHRVGDADFRVRPVHAIRGTWGAEFISELTPEEDKGDYVVQKRRHSAFSYTDMDLFLREEKIDTVVLTGVWTNVCVRSTASDALYHGYNVITISDGTASKDQSMHESGLRDIGIFGEIYSSDEYIEAFKKSLTNQQSAS

SEQ ID NO: 84 - protein, 186 aa, Paenibacillus borealis
  4-ureido-5-carboxyl-imidazole-amide hydrolase
MSKHAVVIIDMLNDFIHPDGALTCPNGAAIVPAIQELIEFSHENDIQVIFVQEAHRQNDADFRVRPVHAIKGTWGSDFITELTPQPDKGDYIVQKRRHSAFSYTDMDLFLREEGIDTVVVTGVWTNVCVRSTASDAMYHTYKVKCISDCCASKDEEMHVSGLRDIGIFGEVLTLAEYKEVNAVVKG

SEQ ID NO: 85 - protein, 200 aa, Thermohalobacter berrensis
  4-ureido-5-carboxyl-imidazole-amide hydrolase
MPRHAILVIDMLNDFVGEKAKLRCPGADDIVPNLQKLFKWVRGREDDNVQIVHIQEAHRKNDADFRVRPVHAVKGTWGSDFIKELYPEEDEYIVQKRRHSGFAYTDLDLYLREENIDTVVVTGVWTNVCVRSTASDALYRAYKVITLSDGCASKTPEMHEYGLKDLSLFSKVMTIDEYIDAWEKGIDPWLGGGDRENKVE

SEQ ID NO: 86 - protein, 200 aa, Tindallia magadiensis
  4-ureido-5-carboxyl-imidazole-amide hydrolase
MPRHAILVIDMLNDFVGEKAPLRCPGGDDIIEPLQKLFKWVRSRQDDDIHLVHIQEAHRKHDADFRVRPVHAVDGTWGSDFIPALYPEGDEYIVKKRRHSGFAHTDLDLYLKEENIDTVVVTGVWTNVCVRSTATDALANAYKVITLSDGCASKTDAMHEHGLVDLSIFTKVMSIDEYIDAWNKGEDPWIGGGDAENKVE

SEQ ID NO: 87 - protein, 197 aa, Ornithinibacillus halophilus
  4-ureido-5-carboxyl-imidazole-amide hydrolase
MVQLKGKKHALVIIDMLNDFIGENGALRCENGEKIVPKIREAIDFCHENGIQVVHIQEAHRKNDADFRVRPVHAIKGTWGSQFIPELEPDEEKGDYVVQKRRHSAFSYTDFDLFLREESIDTVVLTGVWTNVCVRSTASDALYHGYNVIALSDGTASKDDDMHRSGLRDMDLFGEIMTTAEYMEAAKKKFSQNQQIV

SEQ ID NO: 88 - protein, 186 aa, Paenibacillus sp.
  4-ureido-5-carboxyl-imidazole-amide hydrolase
MGKHAVVIIDMLNDFIHPDGALTCPNGAGIVPALQELIEFSHENDIQVIFVQEAHRKNDADFRVRPVHAIKGSWGSDFIAELTPDEAKGDYIVQKRRHSAFSYTDMDLFLREEGIDTVVVTGVWTNVCVRSTASDAMYHTYKVVCVSDCCASKDEEMHISGLRDIGIFGEVLTLAEYKEKNLVVRA

SEQ ID NO: 89 - protein, 196 aa, Bacillus oceanisediminis; identical to SEQ ID NO: 10
  4-ureido-5-carboxyl-imidazole-amide hydrolase

SEQ ID NO: 90 - protein, 203 aa, Soehngenia sp.
  4-ureido-5-carboxyl-imidazole-amide hydrolase
MAKHAILVIDMLNDFADPNGVLYCPNSRKIIPTMQKLFNWVRERNKNGNDDIQLIFIQEAHRKNDADFKVRPVHAVKGTWGSDFIDELRPLENEYIVQKRRHSAFAHTDLDLYCREEKIDTIVVTGTWTNVCVRSSASDALYYAYKVITLSDAVQSKTEEMHQSGLRDLSIFTKVMTVDEYMEAWDKGLDPWEGGGDKENKVE

SEQ ID NO: 91 - protein, 204 aa, Soehngenia saccharolytica; identical to SEQ ID NO: 73
  4-ureido-5-carboxyl-imidazole-amide hydrolase

SEQ ID NO: 92 - protein, 203 aa, Proteiniborus ethanoligenes
  4-ureido-5-carboxyl-imidazole-amide hydrolase
MARHAILIIDMLNDFANEKGALYCEGTAKITPNLQKLFKWVRERNAQGKDDVQLVHIQEAHREKDADFRVRPVHAIKGTWGSDFIKELYPEQGEYIVQKRRHSAFWHTDLDLFLREEAIETVVVTGVWTNVCVRSTAADALANAYKVITLSDGVHSKTDEQHEHGLVDLSIFTKVMTIDEYTDAFEKGLDPWVGAGDPENKVK

SEQ ID NO: 93 - protein, 200 aa, Anaeromicrobium sediminis
  4-ureido-5-carboxyl-imidazole-amide hydrolase
MPRHAIVVIDMLNDFVGEKAPLRCPGADDIIPDLQKLFKWVRQRENDDIHLVHIQEAHRKNDADFRVRPVHAVKGTWGSDFIEELYPEGDEYIVPKRRHSGFAHTDLDLYLREENIDTVVVTGVWTNVCVRSTASDALYNAYKVITLSDGCASKTKEMHEYGLNDLSIFTKVMSIDEYIESWEKGLDPWIGGGDKENKVE

SEQ ID NO: 94 - protein, 197 aa, Bacillus freudenreichii; identical to SEQ ID NO: 30
  4-ureido-5-carboxyl-imidazole-amide hydrolase

SEQ ID NO: 95 - protein, 200 aa, Alkaliphilus metalliredigens
  4-ureido-5-carboxyl-imidazole-amide hydrolase
MAKHAILVIDMLNDFVGEKAPLRCPGGETIIPDLQKIFEWVRGREGDDIHLVHIQEAHRKNDADFRVRPLHAVKGTWGSDFIPELYPQEDEYIVQKRRHSGFAHTDLDLYLKEEGIDTVVLTGVWTNVCVRSTATDALANAYKVITLSDGTASKTEEMHEYGLNDLSIFTKVMTVDQYIQAWENDEDPWVGGGDAQNKVK

SEQ ID NO: 96 - protein, 189 aa, Thermoflavimicrobium sp.
  4-ureido-5-carboxyl-imidazole-amide hydrolase
MRGNKHALVIIDMLNDFIGEKATLRCENGEKIVSKIRELIDFCHENDIQVIHVQEWHRKNDADFRVRPVHAIRGTWGADFIPELQPEEDKGDYIIQKRRHSAFSYTDMDLFLREEGIDTVVLTGVWTNVCVRSTGSDALYHGYNVIAISDACASQTNDMHESGLRDIGIFGEVFSTEEYKKLMLEKIGK

SEQ ID NO: 97 - protein, 203 aa, Bacteroidetes bacterium
  4-ureido-5-carboxyl-imidazole-amide hydrolase
MTRHGIVVIDMLNDFIGEKAQLRCPGGDDIIEPLQRLFKYVRERNAKGNDDIQLIHIHEAHRKNDADFRVRPRHAVAGTWGSDFIKELYPEGDEYIIPKRRHSSFAHTDLDLYLREENIDTVVVVGVWTNVCVRSTASDALYNAYKVITISDCCASKTQEMHEYGLKDLSIFTKVMTLDEYIDAWEKGEDPWLGGGDAENKVE

SEQ ID NO: 98 - protein, 193 aa, Ammoniphilus sp.
  4-ureido-5-carboxyl-imidazole-amide hydrolase
MKGNKHAVLVIDMLNDFIGPNAALRCEAGEEIVPNIRKMIDFAHDKGIQVVHIQEAHRKNDADFRVRPVHAIKGTWGSQFIKELEPEEDKGDYVVQKRRHSGFSYTDLDLFLREEKIDTVVLTGVWTNVCVRSTASDALYRGYNVICLSDGTASQTENMHEAGLRDMSLFGVVMSTEEYMLEAESKFENRDSA

SEQ ID NO: 99 - protein, 203 aa, Gottschalkia purinilytica
  4-ureido-5-carboxyl-imidazole-amide hydrolase
MPRYGIVVIDMLNDFIGEKAPLRCPGGEEIVPNLQKLFKWVRERNAQGKDDVQLIHIQEAHRKNDADFRVRPVHAVKGTWGSDFIKELYPEGEEYTIPKRRHSSFWHTDLDLYLREENLDTVVVTGVWTNVCVRSTAADALALAYRVITLSDGVHSKNQAQHEHGLVDLSIFTKVMTIDEFTSAIDAGKDAWAGAGDQENKVE

SEQ ID NO: 100 - protein, 198 aa, Bacillus bacterium
  4-ureido-5-carboxyl-imidazole-amide hydrolase
MTQQLKGEKHALVIIDMLNDFIGEKAALRCENGEKIVPKIREAIDFCHENGIQVVHVQEAHRKNDADFRVRPVHAIKGTWGSEFIEELQPEEDKGDYIVQKRRHSAFSYTDFDLFLREEKIDTVVLTGVWTNVCVRSTASDALYHGYNVIALSDATASQDDDMHRGGLRDIGIFGDVMTTDEYMDAAKKKFSQDPQTV

SEQ ID NO: 101 - protein, 269 aa, Paenibacillus sp.
  4-ureido-5-carboxyl-imidazole-amide hydrolase
MKMSHGDTIPLHPHLSSQSDIDEIEILMNSSPATVLPATPPRATIPISPSPPNLPPPPPPKPSPPSNHNHKPASQPLPSRPELPPSGFGSAPNTLTEPVWDTVKRDLSRIVSNLKLVVFPNPFREDPGKALRDWDLWGPFFFIVFLGLILSWSASVKKSEVFAVAFALLAAGAVILTLNVLLLGGHIIFFQSLSLLGYCLFPLDVGALICMLKNNVIVKIVVVSVTLAWSSWAAYPFMSSAVNPRRKALALYPVFLMYVSVGFLIIAID

SEQ ID NO: 102 - protein, 203 aa, Paraclostridium bifermentans; identical to SEQ ID NO: 43
  4-ureido-5-carboxyl-imidazole-amide hydrolase

SEQ ID NO: 103 - protein, 485 aa, Bacillus firmus
  4-ureido-5-carboxyl-imidazole-amide hydrolase
DLILKNASIPQGDRQVLTNILVNEGKIVGYTNDISFLNAARVIDIKGKLVVPGCIDPHTHFMDPGFTHRETFATGSRSAAAGGLTTIIDMPCCSKPSVRDGESFNKKIGPIRDQAYIDYCFWGGMTGEDAREGWIDNIYPQIDQGVVAFKVYMTPSVPTFPKVSDAEMLEIFSNVAKTGLPIGVHAENYDICTFYSKKLEKEGRLDGPAWAEARSSLAEKVAIELILSFAEATDARVHVVHMSTKEGVELVKAAKKRGVKVTAETCPHYLVLNAKDSMSERGSYAKIAPPLRGKEDNESHWQALADGTIDFVGTDHAPYEIATEKDYPGATIWNTFPGIPGVETMVPILVSEGLNKGRLSLSRFVEVTSRNAAIHYGIYPKKGSMEIGSDADFTVIDLEKEYVIDEQKTESMAKYNPLHGMKLKGKPVQTIIRGNVIFDEDKGIVGEAGYGEYVPRQSIQRLERTFKYEKYEAVKAKTKDPVAQQ

SEQ ID NO: 104 - DNA, 44 bp, Artificial Sequence
  primer
cctgtacttccaatccaatgatttaattttgaaaaatgcaagca

SEQ ID NO: 105 - DNA, 37 bp, Artificial Sequence
  primer
ccgttatccacttccaatttattgctgtgctactggg

SEQ ID NO: 106 - protein, 531 aa, Clostridium purinilytica
  4-ureido-5-carboxyl-imidazole-amide hydrolase
MYDLILKNARIPQGDDTIVTNILVRDEKIAGFTDCIEEVQAKEIIDIEGQLTLPGCIDSHTHFMYQGFPHRENFLTGTAAAATGGITTVIDMPCCSVPSARSVEQLQLKIDLCQPQSLVDFAMWGGVTGEDVREDWMHNVKEQADYGVVAFKVYMTPSVPTYPRVTDPEMLECFRAVAETGLPIGIHAENFAMCDFYVKKFQKEGRMDGPAWAEARMELAEKVAIELGISFAEDTGARLHIVHMSTGIGAKLVGEAKKRGINVTSETCPHYLTLNYQDAMSEYGAFAKIAPPLRTKRDNEELWEGLNNGSVDFIATDHAPYEIESEKAKEGMNIWTAFPGIPGVETMVPVLVSEGYNKGRLSLSKLVDILSTNAAKHYGLYPKKGALNIGSDADFTVIDLDREWTITKDMVTMCGYNPLEGLKLKGKPVKTIVRGHIVYQDGEEGALPSLTDEELKTIVHEYPAGVEEKYSGVFEGFPHLHSAEYERSYRKEHPEVIDEHIRGIKGIMVKPGFGQFVKRQSIQVLPKTITY

SEQ ID NO: 107 - protein, 472 aa, Thermoflavimicrobium sp.
  4-ureido-5-carboxyl-imidazole-amide hydrolase
MQLILKNARIPQGQGNDTVITNILVNENKIAGFVNHVDGIEAQQVIDLEGKLVVPGCIDPHTHFMDPGFTHRETFETGSASAAAGGLTTIVDMPCCSKPSVRSKDEFHAKWDKVKNNSYIDFGSWGGMTGEDVREDKLDNVWGQVEEGITCFKVYMTPSVPTFPKVTDAEMLEIFAKVAETGLPVGIHAENFDICDFRVKKLRKEGRMDAPAWSEARNNLAEKVAIELVISFAEATGARAHIVHMSTKEGVELVRQAKLRGVPITAETCPHYLVLNYKDALTEFGTFAKVAPPLRETADNEAHWKALADGTIDFVATDHAPYEIPIEKVGEGLNIWDSFPGFPGVETMVPVLVSEGLNKGRLTLQRFVEVTSTNGAILHGLYPQKGHMGMGADADFTVIDLEREYTIDKDKVHTMAKYTPFHGMKLKGKPIMTIVRGTVVYDEDRGGLVGQVGYGQYVRRQTNIHPPRQIQF

SEQ ID NO: 108 - protein, 473 aa, Marininema halotolerans
  4-ureido-5-carboxyl-imidazole-amide hydrolase
MDLLLKNANIPQGDRQVQTNILVDEGKIVGFVQDASGVMAERTIDLTGKLVVPGCIDPHTHFMDPGFTHRETFAEASMSAAAGGLTCHVDMPCCSKPSVRSEDELYMKLNPIKDQAYIDFAFWGGMTGEDVREGKLDNVWGQVEAGVTAFKVYMTPSVPTFPKVSDAEMLEIFQKVAETGLPVGVHAENYDICSFYANKLEKEGRTDSPAWAEARMALAEKAAIQLIISFAEETGARAHIVHMSTKEGAALIRDAKRRGVKITAEACPHYLTLNAKEHMTQFGNLAKIAPPLRGKEDNEVHWQALSDGTIDFVGTDHAPYEIATEKVADGLTIWNSFPGIPGVETMVPVMVSEGLNKGRLSLSRFVEVTSRNAAIHFGLYPKKGSMELGSDADFTVIDLDREYVIDQEKTLTKAKYTPFHGMKLKGKPVMTIVRGGVVYDEDHGGIQATAGYGQFVKRQSNIELKRVEGSLIY

SEQ ID NO: 109 - protein, 470 aa, Unknown
  Clostridiaceae bacterium 4-ureido-5-carboxyl- imidazole-amide hydrolase
MSIDLLIKNAMIPKGDTTVKVDILVDKGIIVGFMEQAQIQAKEIIDACEKLVIPGCIDSHTHINDPGFTHRENFLTGTSAAASGGVTTIIDMPCCSVPSVRSVDNLEYKLSQIADKAIVDYGMWGGVTGEDVRNNWLHNVKEQSDYGVCAFKVYMTPSVPTYPRVTDPEMLEAFKSVAATGLPIGIHAENYAMCDFYVQKLKNEGRLDGPAWAEARLELAEKVAIELGISLAEFTGARLHIVHMSTGIGAVLVKNAKMRGLDVTSETCPHYLTINYQDAMSKYESFAKIAPPLRTVKDNEMLWEGVADGSVDFIATDHAPYEISTEKCAEGMNIWNSFPGIPGVETMVPVIVSEGYNKGKISLSRMVEILCKNPAIHYGLYPKKGSMEIGSDADFTIIDLDKEWTIEQEKMYTMAKYTPLHGMTLKGKPVKTIVRGSLVYEDGKGIIAKPGYGRFVKRQTISKLPRQIKF

SEQ ID NO: 110 - protein, 486 aa, Bacillus sp.
  4-ureido-5-carboxyl-imidazole-amide hydrolase
MDLILKNASIPQGDRQVLTNILVNEGKIVGYTNDISFLNAARVIDIKRKLVVPGCIDPHTHFMDPGFTHRETFATGSRSAAAGGLTTIIDMPCCSKPSVRDGESFNKKIGPIRDQAYIDYCFWGGMTGEDAREGWIDNIYPQIDQGVVAFKVYMTPSVPTFPKVSDAEMLEIFRNVAKTGLPIGVHAENYDICTFYSKKLEKEGRLDGPAWAEARSSLAEKVAIELILSFAEATDARVHVVHMSTKEGVELVKAAKKRGVKVTAETCPHYLVLNAKDSMSERGSYAKIAPPLRGKEDNESHWQALADGTIDFVGTDHAPYEIATEKDYPGATIWNTFPGIPGVETMVPILVSEGLNKGRLSLSRFVEVTSRNAAIHYGIYPKKGSMEIGSDADFTVIDLEKEYVIDEQKTESMAKYNPLHGMKLKGKPVQTIIRGNVIFDEDKGIVGEAGYGEYVPRQSIQRLERTFKYEKYEAVKAKTKDPVAQQ

SEQ ID NO: 111 - protein, 470 aa, Paenibacillus sp.
  4-ureido-5-carboxyl-imidazole-amide hydrolase
MGYELIIKNASIPQGDRQVLTDILVNKGVIVGFAKDIDAEADTIIDAQGKLVLPGVIDSHTHFNDPGFTHREDFVTGTSSAAAGGVTMIVDMPCCSVPSVRSVENLHNKLNALEGKGIVDYAMWGGITGEDVRNDVLDIVKEQAAEGVVAFKVYMTPSVPSYPRVTDPEMLEAFYAVAETGMPIGIHAENYAICDYYTQKLKKEGRMDYPAWAEARKILAEKAAIQLGISFSEESGARLHIVHMSTAAGNQLIKEAKLKGVKVTAESCPHYLTLNAVDAMTEFGTFAKIAPPLRRTEDNEALWQGLVDGTIDFMATDHAPYEIATEKDAPGMDVWTSFPGIPGVETLVPIMISEGYNKGRLSLSRLVELLCTNPAIHYGLYPKKGALEIGTDADFTIVDLEKEWTLDKDKMHCKPKYTPFHGMKLKGKVDKTIVRGTLVYDDELGVVGKPGYGEFVKRQTISELPRLLKY

SEQ ID NO: 112 - protein, 472 aa, Thermoflavimicrobium dichotomicum
  4-ureido-5-carboxyl-imidazole-amide hydrolase
MQLILKNATIPQGFGNRTVKTNILVDDNKIVGFTNCIDGIEAKEVIDMDGKLVVPGCIDPHTHFMDPGFTHRETFATGSASAAAGGLTCIVDMPCCSKPSVRSRDEFYQKLNPIKDQSYIDFAFWGGMTGEDVREGKLDNVWGQVEEGVTAFKVYMTPSVPTFPKVSDAEMLEIFAKVAETGLPVGVHAENYDICSFRVEKLKKQGRMDGPAWAEARNSLAEKVAIELVISFAEATGARAHIVHMSTKEGVGLVRQAKLRGLPITAETCPHYLVLNAQEAMTEFGTFAKIAPPLRGKEDNEVHWKALADGTIDFVATDHAPYEIPTEKEGEGLDIWTAFPGIPGVEQMVPILVSEGLNKGRLTLQRFVEVTSTNAAIHFGLYPKKGSMEIGTDADFTVIDLEREYVIDKDKVHSMAKYTPFHGMKLKGKPVMTIVRGTVVYDEDHGGLVGKKGYGQFVKRQSNIALPRQLQF

SEQ ID NO: 113 - protein, 490 aa, Fictibacillus enclensis
  4-ureido-5-carboxyl-imidazole-amide hydrolase
MDLILKNAMIPQGDRTVITNILVHEGRITGFCNDIQTLNADKVLDMEGKLVVPGCIDPHTHFMDPGFTHRETFATGSRSAAAGGLTTIIDMPCCSKPSVRDGESFQKKIAPIRGQAYIDYCFWGGMTGEDARNGWIDNIYPQIDEGVVAFKVYMTPSVPTFPKVSDGEMLEIFQKVAKTGMPIGVHAENYDICTFYTEKLKNEGREDGPAWAEARMALAEKVAIELIISYAEATDARVHVVHMSTKEGVKLVREAKKRGVKVTSETCPHYLMLNAQDSMTERGTFAKIAPPLRGKDDNQAHWQALADGTIDFVGTDHAPYEIATEKAAADSSIWNSFPGIPGVETMVPILVSEGLNKGRLSLSRFVEVTSKNAAVHYGIYPKKGAMEIGSDADFTVIDLDHEWVIDEKQTESMAKYNPLHGIKLKGRPVRTILRGTVVYDYEKGGITGTAGYGEYVKRQSIQRLERMLKYENYETANQPSAARQQLEKSK

SEQ ID NO: 114 - protein, 473 aa, Marininema mesophilum
  4-ureido-5-carboxyl-imidazole-amide hydrolase
MDLLLKNANIPQGDRQVQTNILVDGGSIVGYVQDASGVIAERTIDLDGKLVVPGCIDPHTHFMDPGFTHRETFAEASMSAAAGGLTSHVDMPCCSKPSVRSEDELYMKLNPIKDQAYIDFAFWGGMTGEDVREGKLDNVWGQVDAGVTAFKVYMTPSVPTFPRVGDAEMLEIFRKVAETGLPVGVHAENYDICSFYANKLEKEGRTDSPAWAEARMALAEKAAIQLIISFAEETGARAHIVHMSTKEGAVLIREAKRRGVKITAEACPHYLTLNAEEHMTKFGALAKIAPPLRGKEDNEVHWQALADGTIDFVGTDHAPYEIATEKIAEGLHIWNSFPGIPGVETMVPIMVSEGLNKGRLSLSRFVEVTSRNAAIHFGLYPKKGTMELGSDADFTVIDLEREYEIDQEKTLTKAKYTPFHGMKLKGKPVMTIVRGGVVYDEDRGGIVGTAGYGQFVKRQSNIELKRVEGSLVY

SEQ ID NO: 115 - protein, 470 aa, Paenibacillus sp.
  4-ureido-5-carboxyl-imidazole-amide hydrolase
MGYELIIKNASIPQGDRQVLTDILVNNGVIVGFAKDIDAEADTIIDAQGKLVLPGVIDSHTHFNDPGFTHREDFVTGTSSAAAGGVTMIVDMPCCSVPSVRSVENLHNKLNALEGKGIVDYAMWGGITGEDVRNDVLDIVKEQAAEGVVAFKVYMTPSVPSYPRVTDPEMLEAFYAVAETGMPIGIHAENYAICDYYTQKLKKEGRMDYPAWAEARKILAEKAAIQLGISFSEETGARLHIVHMSTAAGNQLIKEAKLKGVKVTAESCPHYLTLNAVDAMTEFGTFAKIAPPLRRPEDNEALWQGLVDGTIDFMATDHAPYEIATEKDAPGMDVWTSFPGIPGVETLVPIMISEGYNKGRLSLSRLVELLCTNPAIHYGLYPKKGALEIGTDADFTIVDLEKEWTLDKDKMHCKPKYTPFHGMKLKGKVDKTIVRGTLVYDDELGVVGKPGYGEFVKRQTISELPRLLKY

SEQ ID NO: 116 - protein, 471 aa, Paenibacillus typhae
  4-ureido-5-carboxyl-imidazole-amide hydrolase
MSYDLIIKNANIPQGDRQVLTDILVNNGVIVGFAKDIDAEAAETIDAGGKLVLPGVIDSHTHFNDPGFTHREDFVTGTSSAAAGGVTMIVDMPCCSVPSVRSADNLFNKLNALEGKGIVDYAMWGGITGEDVRNNVLPEIISEQVANGVVAFKVYMTPSVPSYPRVTDPEMLEAFAAVSPTGLPIGIHAENFAICDYFTNKLKGEGRLDYPAWAEARMALAEKAAIQLGISFAEETGARLHIVHMSTGAGNELIREAKLKGTKVTAESCPHYLTLNAVDAMTEFGTFAKIAPPLRTAKDNEVLWQGLADGTIDFMATDHAPYEIATEKDAPGMDVWTSFPGIPGVETLVPIMISEGYNKGRLSLSRLVELLCTNPAVHYGLYPKKGALEIGTDADFTIVDLEKEWTLDKDQMHCKPKYTPFHGMKLKGKVDKTIVRGTLVYDEALGVVGKPGHGEYVKRQTISELPRLLKY

SEQ ID NO: 117 - protein, 532 aa, Tissierella praeacuta
  4-ureido-5-carboxyl-imidazole-amide hydrolase
MYDVIVKNARIPQGDDTALTNILVKDEKIAGFVDNIEGIEAKEIIDAKGHLTLPGCIDSHTHFMYQGFPHRENFLTGTAAAARGGVTTVIDMPCCSVPSARSKEQLELKLDLIQPQALVDFALWGGVTGEDVREGWLDHVQEQADWGVVAFKAYMTPSVPTFPRVTDPEMFEAFKAVAKTGLPVGIHAENYSMCDYYVKEFQKQGRMDGPAWAEARMELAEKVAIELGISFAEEAGARLHIVHMSTGIGAKLVGEAKKRGLNVTSETCPHYLTINAQEAMTKYGACAKIAPPLRTKKDNELLWEGLNNGSVDFIATDHAPYELESEKIKEGTNIWTAFPGIPGVETMVPVLVSEGYNKGRISLSTLVDVLSKNAAVHYGLYPKKGAMNIGSDADFTIIDLDKEWVIDPKNEASMCGYTPLEGIKLKGKVAKTIVRGHLVFEDLDESTLAKLTDEELKKIVHNFPEGIEEKYAEQFKKYPHLYSAEYERSYRINHPEVIDEHIRGIKGIVAKPGFGKFVKRQSIQILPRKIKY

SEQ ID NO: 118 - protein, 495 aa, Bacillus sp.
  4-ureido-5-carboxyl-imidazole-amide hydrolase
MDLILKNANIPQGDRQVLTNILVNDGKITGFVSNITGLVADKVIDCKGYLVVPGCIDPHTHFMDPGFTHRETFATGSRSAAAGGVTTIIDMPCCSKPSVRDKESFHKKIGPIRDQAYIDYCFWGGVTGEDVREGWLDNIYPQIEEGVVAFKVYMTPSVPTYPKVSDGEMLEIFRKVAPTGLAIGVHAENYDICTFDTEKLKKAGRLDGPAWAEARSSLAEKVAIQLIISYAEATDARVHVVHMSTKEGVELVREAKKRGVKVTSETCPHYLMLNAQDAMSERGTFAKIAPPLRGKEDNEEHWQALADGTIDFVGTDHAPYEIATEKAAADSDIWNSFPGIPGVETMVQILVSEGLNKGRLSLSRFVEVTSRNAAVHYGIYPKKGAMEIGSDADFTIIDLNREYTIDEQKTESMCKYNPLHGLQLKGKPVQTVIRGELIYDEDNGGIVGKAGYGEYVKRQSIQRLDRVLKYETYEAKGNDAADQKEAVLSQSSDRK

SEQ ID NO: 119 - protein, 469 aa, Bradyrhizobium japonicum
  4-ureido-5-carboxyl-imidazole-amide hydrolase
MDLILKNANIPQGDRQVLTNILVENERIVGFVNSIDHIEAKQAIDVNGKLVVPGCIDPHTHFMDPGFTHRETFATGTMSAAAGGVTTIIDMPCCSKPSVRSDETLFAKLNPLLEQAHIDFCFWGGMTGEDVREGWLDNVWGQVEQGVVAFKVYMTPSVPTFPRVSDAEMLEIFYKVAETGLPVGVHAENYDICSFYTEKLKKQGRMDGPAWAEARKALAEKVAIELIISYAEETGARVHVVHMSTKEGAHVVREAKKRGVKVTAETCPHYLMLNAEEAMTEHGTFAKIAPPLRGKEDNEVHWKALADGTIDFVGTDHAPYEIATEKAAEDSDIWNSFPGIPGVETMVPILVSEGLNKGRLSLSRFVEVTSRNAAIHYGIYPKKGAMEIGSDADFTVIDLEKEHTIDQAQTYSMAKYNPLHGIQLKGMPVMTIVRGTVVYEDGKGIVGKPGYGKFTKRESIQQLDRTLKY

SEQ ID NO: 120 - protein, 486 aa, Bacillus firmus
  4-ureido-5-carboxyl-imidazole-amide hydrolase
MDLILKNANIPQGDRKVLTNILVNEGKIVGYTNDISFLNAAKVIDIQGKLVVPGCIDPHTHFMDPGFTHRETFATGSRSAAAGGLTTIIDMPCCSKPSVRDGDSFNKKIGPIRDQAYIDYCFWGGMTGEDAREGWIDNIYPQIDQGVVAFKVYMTPSVPTFPKVSDAEMLEIFKNVAKTGLPIGVHAENYDICTFNSKKLEKEGRLDGPAWAEARSSLAEKVAIELILSFAEATDARVHVVHMSTKEGVELVKAAKKRGVKVTAETCPHYLVLNAQDSMSERGSYAKIAPPLRGKEDNESHWQALADGTIDFVGTDHAPYEIATEKDYPGATIWNTFPGIPGVETMVPILVSEGLNKGRLSLSRFVEVTSRNAAIHYGIYPKKGSMEIGSDADFTVIDLEKEYVIDEQKTESMAKYNPLHGMKLKGKPVQTIIRGNVVFEEDKGIVGEAGYGEYVPRQSIQRLERTFKYEKYEAVNVKTKDPVAQQ

SEQ ID NO: 121 - protein, 512 aa, Acidaminobacter hydrogenoformans
  4-ureido-5-carboxyl-imidazole-amide hydrolase
MKYDLIVKNALIPAMNGNDTLMTNILVKNEKIAGFLESVEGLEAERYVDAGGKLVLPGCIDSHTHYMDPGFTHRETFLTGTSQAAAGGLTTIIDMPCCSFPRSVLDVDSMNEKLDAIKPQAIVDFGLWGGATGDMVRDGKLADVQAQADAGVLAFKVYMTPSVPTFNRVTDPEMHEVFKAVEKTGVAVGVHAENFAMCDYYVRKYQAEGRLDGPAWSEARLELAEKAAIQLGIAFAEETGARYHVVHMSTGMGARLIGEAKKRGLKVTSEVCPHYLVLNAPEAMSEYGAYAKIAPPLRTTQDNIELWEGLANGSVDFVATDHAPYKIQANKDNDDAAVEKDAPGMNIWTAFPGISGSETMVNIIISEGYNKGRLTLERTVEVLSKSAAIHYGLYPKKGSMNIGSDADLTIIDLEKEWAIDKDVSFMKNKYTPLHGLKLKGKVAHTILRGTSVYHEEPNEVQGKILVEPGFGQFVKRQSIQVLPNKLQFENYKAIGADFFNNYHRRNINNVID

SEQ ID NO: 122 - protein, 470 aa, Caloranaerobacter sp.
  4-ureido-5-carboxyl-imidazole-amide hydrolase
MYDVIIKNARIPQGDDTILTNILVKDEKIAGFTQDLDGIDAKEIIDAEGHLTLPGCIDSHTHINDPGFTHRENFLTGTSAAASGGITTIIDMPCCSVPSVRSVENLEHKLEAIKDKAIVDYAMWGGVTGEDVRNGWLHNVEEQADYGVCAFKVYMTPSVPTYPRVTDPEMLEAFKAVAKTGLPIGIHAENFAMCDFYVKKFQAEGRMDGPAWAEARMTLAEKVAIQLGISFAEETGARLHIVHMSTGIGAILVGEAKKRGLDVTAETCPHYLTLNYKDAMSEFKQFAKIAPPLRTKEDNEILWKALQDGRVDFIATDHAPYEIPTEKEAEGMNIWTAFPGIPGVETMVPVIISEGYNKGRLSLSRLVEVLSTNPAKHYGLYPKKGAMFIGSDADFTIIDLEKEWTIDKDKMYTMAKYTPFHGFKLKGKPVKTVVRGKLVYDDTEGIVGREGYGKFIKRQNKGKLERLIKF

SEQ ID NO: 123 - protein, 532 aa, Tissierella sp.
  4-ureido-5-carboxyl-imidazole-amide hydrolase
MYDIIVKNARIPQGDDTIITNILVKDEKIAGFLDNIEGIAAKEIIDAEGHLTLPGCIDSHTHFMYQGFPHRENFLTGTAAAASGGVTTIIDMPCCSVPSVRSAKQLQLKVDLIQPQALVDFALWGGVTGEDVREGWLDHVQEQADYGVVAFKAYMTPSVPTFPRVTDPEMYEAFKAVAKTGLPVGIHAENYSMCDYYVKEFQKQGRMDGPAWAEARMELAEKVAIELGISFAEDAGARLHIVHMSTGIGAKLVGEAKKRGLNVTSESCPHYLTLNYQESMTKYGAFAKIAPPLRTKKDNEEQWEGINNGSIDFIATDHAPYEIESEKAREGMNIWTAFPGIPGVETMVPIIVSEGYNKGRISLSKLVEILSTNAAIHYGLYPKKGAMHIGSDADFTIIDLDKEWVIEPKKQASMCGYSPLEGMKLKGKVAKTIVRGHLVFEDIEESVLAGLTDEELKTIVHNFPEGIDEKYADEFSKYPYLHSAEYENSYRINHPEVIDKHIREIKGIKAKPGFGQFIKRQSIQILPRKITF

SEQ ID NO: 124 - protein, 497 aa, Bacillus sp.
  4-ureido-5-carboxyl-imidazole-amide hydrolase
MDLILKNALIPQGDRKVKTNILVDDGKIVGFTSALNGLSADRVIDMKENLVVPGCIDPHTHFMDPGFTHRETFTTGTRSAAAGGVTTHIDMPCCSKPSVRDAESFHKKLAPLKDQAYIDFAFWGGMTGEDVREGLLDNIQPQIDEGVVAFKVYMTPSVPTFPKVSDAEMLEIFSHIAPTGMPIGVHAENYDICTFYSEKLKKEGRLDGPAWAEARMALAEKAAIQLIISFAEATGARVHVVHMSTKEGVELVRAAKKRGVKVTAETCPHYLMLNAEDSMSERGTFAKIAPPLREKEDNIVHWQALADGTIDFVGTDHAPYEIPTEKDAPDSTIWNSFPGIPGVETMVPILVSEGLNKGRLSLSRFVEVTSRNAAIHFGIYPKKGAMEIGSDADFTVIDLEREYTIDEQKTESMAKYNPLHGLKLKGKPIQTIVRGRVVYDEDNGGIMGEAGYGQYVKRQSIQSLERVIKYETFEEGAKEAEAKPRMEKALKRDMITN

SEQ ID NO: 125 - protein, 470 aa, Paenibacillus donghaensis
  4-ureido-5-carboxyl-imidazole-amide hydrolase
MSYELVIRNASIPQGDRQVLTDILVDKGVIVGFARDFEPGDAEVIDAKGMLVLPGVIDSHTHFNDPGFTHREDFVTGTSSAAAGGVTMIVDMPCCSVPSVRSVDNLHSKLNALEGKGIIDYAMWGGVTGEDVRNDVLNIVKEQAAEGVVAFKVYMTPSVPTYPRVTDPEMLEAFYAVAETGMPIGIHAENFAICDFYTNKLKKEGRTDYPAWAEARKILAEKAAIQLGISFSEESGARLHIVHMSTAAGNGLIKAAKLKGVKVTAETCPHYLTLNAVDAMTEFEAFAKIAPPLRRPEDNESLWQGLVDGTIDFMATDHAPYEIATEKDAPGMDVWTSFPGIPGVETLVPIMISEGYNKGRLSLSRLVELLCTNPAVHYGLYPKKGAIEIGTDADFTIVDLEKEWTLDKDQMHCKPKYTPFHGMKLKGKVDKTIVRGTVVYDDELGVVGKPGYGEFVKRQTISELPRLLKY

SEQ ID NO: 126 - protein, 555 aa, Gottschalkia acidurici
  4-ureido-5-carboxyl-imidazole-amide hydrolase
MSYDVIIKNARIPQGDNTVLTNILVKDEKIAGFINCIDGIEAKNIIDAEGNLTAPGFIDSHTHYMYQGFPHRENFLTGSAASARGGITTVVDMPCCSVPSARSVDQLKLKIDVCAPQAVIDYAMWGGVTGEDVREGWLHNVKEQADYGVVGFKVYMTPSVPTYPRVTDPEMLEIFKAVAKTGLPVGVHAENFAICDYYVKKFQEEGRMDAPAWSEARNELAEKVAIELGISFAEATGARYHVVHMSTGIGAKLVADAKRRGINVTAESCPHYLTLNYQDTLTKFGSFAKVAPPLRTSADIEEQWEGLRDGSIDFMATDHAPYEIESEKKKEGMNIWTAFPGFPGTETMAPIIISEGLNKGRLSLSRTIEVLSTSAAKQYGLYPKKGALEIGSDADFAIVDLDKEWVIDSKENQASMCGYTPLEGMKLKGRIVKTIVRGNIVFDDTQEGVLPSLTDEEMKTIVHNYPAGVEEDYADIFEQFPNLHSAEYDRSYRKEHPEKIDEDVRRIKGIMVKPGFGQLVKRQSIQELPKTLTYDVKAPTGINRRERESLPEFNK

SEQ ID NO: 127 - protein, 497 aa, Bacillus fortis
  4-ureido-5-carboxyl-imidazole-amide hydrolase
MDLILKNALIPQGDRKVKTNILVDDGKIVGFTSALNGFSADRVIDMKENLVVPGCIDPHTHFMDPGFTHRETFTTGTRSAAAGGVTTHIDMPCCSKPSVRDAESFHKKLAPLKDQAYIDFAFWGGMTGEDVREGLLDNIHPQIDEGVVAFKVYMTPSVPTFPKVSDAEMLEIFSHIAPTGMPIGVHAENYDICTFYSEKLKKEGRLDGPAWAEARMALAEKAAIQLIISFAEATGARVHVVHMSTKEGVELVREAKKRGVKVTAETCPHYLMLNAEDSMSERGTFAKIAPPLREKDDNIVHWQALADGTIDFVGTDHAPYEIATEKDAPDSTIWNSFPGIPGVETMVPILVSEGLNKGRLSLSRFVEVTSRNAAIHFGIYPKKGAMEIGSDADFTVIDLEREYTIDEQKTESMAKYNPLHGLKLKGKPIQTIVRGRVVYDEDNGGIVGDAGYGQFVKRQSIQSLERVIKYETYEEGAKEAEAKPRVEKALRRDLITN

SEQ ID NO: 128 - protein, 485 aa, Bacillus oceanisediminis
  4-ureido-5-carboxyl-imidazole-amide hydrolase
MDLILKNANIPQGDRQVLTNILVNDGKIVGYTNDISFLNAKKVIDIAGKLVVPGCIDPHTHFMDPGFTHRETFATGSRSAAAGGLTTIIDMPCCSKPSVRDGESFNKKIAPIRGQAYIDYCFWGGMTGEDAREGWIDNIYPQIDQGVVAFKVYMTPSVPTFPKVSDAEMLEIFSNVAKTGLPIGVHAENYDICTFYSKKLEKEGRMDGPAWAEARSSLAEKVAIELILSFAEATDARVHVVHMSTKEGVELVKAAKKRGVKVTAETCPHYLVLNAKDAMTERGAYAKIAPPLRGKEDNESHWQALADGTIDFVGTDHAPYEIATEKDYPGATIWNTFPGIPGVETMVPILVSEGLNKGRLSLSRFVEVTSRNAAIHYGIYPKKGSMEIGADADFTVIDLNKEYVIDEQKTESMAKYNPLHGMKLKGKPVQTIVRGNIVFEEDKGITGEAGYGEFVPRQSIQRLERTFKYEKYNEVKVTSKETVKQ

SEQ ID NO: 129 - protein, 493 aa, Virgibacillus profundi
  4-ureido-5-carboxyl-imidazole-amide hydrolase
MDLILKNALIPQGDRKVETNILVDNGEIVGFTNAINGMAAKEIIDIKGNLVVPGCIDPHTHFMDPGFTHRETFATGTRSAAAGGITTHIDMPCCSKPSVRDRESFYKKIEPLKSQAYIDYCFWGGMTGEDVREELLDNIYPQIEEGVVAFKVYMTPSVPTFPKVTDAEMLEIFSKIAPTGMPIGVHAENYEICTFYSEKMKKEGRMDGPAWAEARMALAEKAAIQLVISFAEATGARVHIVHMTTKEGVELVREAKKRGVKVTAETCPHYLVLNAQDSMTERGTFAKIAPPLREKEDNIVHWQGLADGSIDFVGTDHAPYEIATEKDAPDSDIWNSFPGIPGVETMVPILVSEGLNKGRLSLSRFVEVTSRNAAIHYGIYPKKGSMELGSDADFTVIDLEREYTIDEQKTESMAKYNPLHGMQLKGMPIQTIVRGKVVYDEDNGGILGESGYGEFVKRQSIQKLDRSIKYEVYEETAKELASQQRQEKALINN

SEQ ID NO: 130 - protein, 471 aa, Paenibacillus sp.
  4-ureido-5-carboxyl-imidazole-amide hydrolase
MSYDLIIKNASIPQGDRQVLTDILVNNGVIVGFAKDFEGDAAEIIDAGGKLVLPGVIDSHTHFNDPGFTHREDFVTGTSSAAAGGVTMIVDMPCCSVPSVRSADNLFNKLNALEGKGIVDYAMWGGITGEDVRNNVLPEIISEQIANGVVAFKVYMTPSVPSYPRVTDPEMLEAFAAVSPTGLPIGIHAENFAICDYFTNKLKGEGRMDYPAWAEARMALAEKAAIQLGISFAEETGARLHIVHMSTGAGNELIREAKLKGTKVTAESCPHYLTLNAVDAMTEFGTFAKIAPPLRTAKDNEVLWQGLTDGTIDFMATDHAPYEIATEKDAPGMDVWTSFPGIPGVETLVPIMISEGYNKGRLSLSRLVELLCTNPAIHYGLYPKKGALEIGTDADFTIVDLEKEWTLDKDQMHCKPKYTPFHGMKLKGKVDKTIVRGTLVYDDALGVVGKPGHGEYVKRQTISELPRLLKY

SEQ ID NO: 131 - protein, 486 aa, Bacillus sp.
  4-ureido-5-carboxyl-imidazole-amide hydrolase
MELILKNANIPQGDRQVLTNILINDGKIVGFSNDISFLNAEKVIDIGGKLVVPGCIDPHTHFMDPGFTHRETFATGSRSAIAGGLTTIIDMPCCSKPSVRDGESFQKKIGPIRDQAYVDYCFWGGMTGEDAREGWLDNVYPQIDEGVVAFKVYMTPSVPTFPRVSDAEMLEIFKAVSKTGLPIGVHAENYDICTFNSKKLEKEGRFDGPAWAEARSTLAEKVAIELIISFAEATDARVHVVHMTTKGGAELVKAAKKRGVKVTAETCPHYLVLNAKDSMSERGSFAKIAPPLRGKEDNEAHWQALADGTIDFVGTDHAPYEIATEKDFPGATIWNTFPGIPGVETMVPILVSEGLNKGRLSLSRFVEVTSRNAAIHYGIYPKKGSMEIGSDADFTVIDLEKEYVIDEQKTESMAKYNPLHGMKLKGKPVQTIIRGNVVYEDGKGVLGQAGYGQYVPRQSIQRLERTFKYEKFEAVKVEAKETVKQN

SEQ ID NO: 132 - protein, 532 aa, Tissierella praeacuta; identical to SEQ ID NO: 117
  4-ureido-5-carboxyl-imidazole-amide hydrolase

SEQ ID NO: 133 - protein, 486 aa, Bacillus oceanisediminis
  4-ureido-5-carboxyl-imidazole-amide hydrolase
MDLILKNASIPQGDRQVLTNILVNEGKIVGYTNDISFLNAARVIDIKRKLVVPGCIDPHTHFMDPGFTHRETFATGSRSAAAGGLTTIIDMPCCSKPSVRDGESFNKKIGPIRDQAYIDYCFWGGMTGEDAREGWIDNIYPQIDQGVVAFKVYMTPSVPTFPKVSDAEMLEIFRNVAKTGLPIGVHAENYDICTFYSKKLEKEGRLDGPAWAEARSSLAEKVAIELILSFAEATDARVHVVHMSTKEGVELVKAAKKRGVKVTAETCPHYLVLNAKDSMSERGSYAKIAPPLRGKEDNESHWQALADGTIDFVGTDHAPYEIATEKDYPGATIWNTFPGIPGVETMVPILVSEGLNKGRLSLSRFVEVTSRNAAIHYGIYPKKGSMEIGSDADFTVIDLEKEYVIDEQKTESMAKYNPLHGMKLKGKPVQTIIRGTVIFDEDKGIVGEAGYGEYVPRQSIQRLERTFKYEKYEAVKAKTKDPVAQQ

SEQ ID NO: 134 - protein, 493 aa, Bacillus sp.
  4-ureido-5-carboxyl-imidazole-amide hydrolase
MDLILKNAMIPQGDRTVITNILVHEGKITGFCSDIHFLKADKVIDMEGKLVVPGCIDPHTHFMDPGFTHRETFATGSRSAAAGGLTTIIDMPCCSKPSVRDGESFQKKIAPIRGQAYIDYCFWGGMTGEDARNGWIDNIYPQMDEGVVAFKVYMTPSVPTFPKVSDGEMLEIFQKVAKTGMPIGVHAENYDICTFYTEKLKKEGREDGPAWAEARMALAEKVAIELIISYAEATDARVHVVHMSTKEGVKLVREAKKRGVKVTAETCPHYLMLNAQDSMSERGAFAKIAPPLRGKEDNQAHWQALADGTIDFVGTDHAPYEIATEKAAADSTIWNSFPGIPGVETMVPILVSEGLNKGRLSLSRFVEVTSKNAAIHYGIYPKKGAMEIGSDADFTVIDLDCEWEIDEKQTESMAKYNPLHGIKLKGRPVQTIVRGNMVYNYEAGGIVGEAGYGEYVKRQSIQRLDRTLKYENYEGAIQTAAPKSGRQQLEKSK

SEQ ID NO: 135 - protein, 471 aa, Anaeromicrobium sediminis
  4-ureido-5-carboxyl-imidazole-amide hydrolase
MNYDVIVKNARIPQGNDTVLTNILVKDEKIVGFTLDLDGIQGNKVIDAEGHLTLPGCIDSHTHINDPGFTHRENFLTGTSAAASGGVTTIVDMPCCSVPSVRSVENLEHKLNAIKNKAIVDYGMWGGVTGEDVRNGWLHNVKEQADYGVLAFKVYMTPSVPTYPRVTDPEMLEAFKAVAETGLPIGIHAENFAMCDFYVKKFEKEGRTDGPAWAEARMTLAEKVAIELGISFAEEAGARLHIVHMSTGVGAKLVGDAKKRGLDVTAETCPHYLTLNYKDAMTEFMQFAKIAPPLRTKSDNEEHWKALQDGRIDFVATDHAPYEIPTEKEAEGMHIWNAFPGIPGVETMVPMIVSEGYNKGRLSLSRLVEVLCSSPAKHYGIYPKKGAMFIGSDADFTIIDLEKEWTIDKDKMYTMAKYTPFHGYKLKGKPVKTVVRGKLVYDDESGIVGEEGYGEFIRRQNIQKLDKAIKY

SEQ ID NO: 136 - protein, 476 aa, Thermosyntropha lipolytica
  4-ureido-5-carboxyl-imidazole-amide hydrolase
MLDTIVKNAMIPQGDELVKANILVEDGKIVGITRKEFEARNVIDAEGNMVLPGCIDSHTHFMDPGFTHRETFTTGCRAAAYGGITTVIDMPCCSVPSVRGVPEMEHKLEAIKPQALIDFALWGGVTGEDVREEKHFRIVREQAEAGVVAFKVYMTPSVPSYPRVTDPEMLETFKAVAETGLPVGIHAENFAMTDFYTQKFIREGRLDAPAWAEARSILAEKVAIELGISFAEEVGARLHIVHMSTGIGARLVGEAKMRGLDVTSETCPHYLVLNYKDAMSEYGAFAKIAPPLRTEEDNEILWEGLQNGSVDFIATDHAPYEIATEKAKQGMTIWTAFPGIPGVETMVPILVSEGYNKGRLTLSQLVDVLSRNAAIHYGLYPKKGAMFIGSDADFTVIDLDREWIINKEDQQSMAKYTPLHGMKLKGKVVKTIVRGKVVFDEKKGIMVNPGYGQFVKRQTISKLDKVIRFPSINDIF

SEQ ID NO: 137 - protein, 471 aa, Alkaliphilus peptidifermentans
  4-ureido-5-carboxyl-imidazole-amide hydrolase
MKYDVVVKNAKIPQGDETVMTHILVKDEKIAGFVTNLDGIEYNSEIDAEGNLTLPGCFDSHTHFMDPGFTHRETFLTGSSSAAAGGLTTIMDMPCCSKPSVRGVEELESKLNAIKDQAVIDYAMWGGVTGEDIRAGKKHIIKEQADYGVCAFKVYMTPSVPTYERVNDPEMLEAFRWVAETGVPVGIHAENFAMCDYFVKKFQKEGRTDGPAWAEARLVEAEQSAIELGINFAKSSGARLHIVHMSSGIGAKLVGEAKKKGLDVTSETCPHYLMLNYQDAMTEHKQFAKIAPPLRTKKDNEELWEGLQNGSVDFMATDHAPYEIETEKDAPGMDIWTAFPGIPGVETLVPILVSEGYNKGRLTLGRLVEVLSSSPAKMYGLYPKKGALMIGSDADFTIIDLEREWTIDQKKTESMAKYNPLHGIKLKGKPVKTVVRGTVVYDDKEGVIGKPGFGQFVKRQRIQKLERTIKY

SEQ ID NO: 138 - protein, 490 aa, Aneurinibacillus migulanus
  4-ureido-5-carboxyl-imidazole-amide hydrolase
MDLILKNANIPQGDRQVQTNILVENGTIVGFVNSINGLQAKNVIDLKGKLVVPGCIDPHTHFMDPGFTHRETFATGTMSAAAGGLTTIIDMPCCSKPSVRSEESFYAKIDPIKDKAYIDYCFWGGMTGEDVREGLLDNVWGQKEAGVVAFKSYMTPSVPTFPKVSDAELLEIFYKVAETGLPVGVHAENYDICSFYTEKLKKEGRLDGPAWAEARKALAEKVAIELIISYAEETGARAHIVHMSTKEGAHLIRQAKERGVKITAETCPHYLVLNAQESMTKYGTFAKIAPPLRGVEDNEIHWQALADGTIDFVGTDHAPYEIPTEKAAEDSDIWNSFPGIPGVETMVPILVSEGLNKGRLSLSRFVEVTSRNAAIHYGIYPKKGAIEIGSDADFTVIDLEKEHVIDQEQTYSMAKYNPLHDIKLKGMPVMTIVRGNVVYEDGKGIVGQEGYGQYVKRQSTQQLDAVINLTKSNESKAVSQSSEKKESVKL

SEQ ID NO: 139 - protein, 470 aa, Paenibacillus sp.
  4-ureido-5-carboxyl-imidazole-amide hydrolase
MGYELIIKNASIPQGDRQVLTDILVNKGVIVGFAKDIDAEADTIIDAQGKLVLPGVIDSHTHFNDPGFTHREDFVTGTSSAAAGGVTMIVDMPCCSVPSVRSVENLHNKLNALEGKGIVDYAMWGGITGEDVRNNVLDIVKEQAAEGVVAFKVYMTPSVPSYPRVTDPEMLEAFYAVAETGMPIGIHAENYAICDYYTQKLKKEGRMDYPAWAEARKILAEKAAIQLGISFSEESGARLHIVHMSTAAGNQLIKEAKLKGVKVTAESCPHYLTLNAVDAMTEFGTFAKIAPPLRRPEDNEALWQGLVDGTIDFMATDHAPYEIATEKDAPGMDVWTSFPGIPGVETLVPIMISEGYNKGRLSLSRLVELLCTNPAIHYGLYPKKGALEIGTDADFTIVDLEKEWTFDKDKMHCKPKYTPFHGMKLKGKVDKTIVRGTLVYDDELGVVGKPGYGEFVKRQTISELPRLLKY

SEQ ID NO: 140 - protein, 516 aa, Marinisporobacter balticus
  4-ureido-5-carboxyl-imidazole-amide hydrolase
MKVDVIVKNARIPQGDDTILTNILVKDEKIAGFTQDLTDVEAGNIIDAQGNLTMPGCIDSHTHFMYQGFGHRENTLTGSAAAATGGVTTVIDMPCCSVPSVRSVEQLNTKVSLWKPQALVDFALWGGVTGEDVREGWLHNVKEQVDAGVVAFKLYMTPSVPTYPRVSDPEMFECFKAVAETGLPVGIHAENFAMCDFYVKKYQEEGRLDGPAWAEARSILAEKTAIQLGISFAEESGARLHIVHMSTGIGAQLVGEAKKRGLSVTAETCPHYLTLNAQEAMTEHGACAKIAPPLRRKEDNEMHWEALRNGSIDFVATDHAPYEIESEKAKAGTNIWTAFPGIPGVETMVPVLVSEGYNKGRISLSRLVEVLSKNAAIHYGLYPKKGAMFIGSDADFTIIDLDRAWTIKQEEQVTLAKYNPLHGMKLKGKPVKTIVRGNLVYSDGEEGVLPSYDNWDVVFNTPDDLQSAEYQVSYRKDHPEKVKKEVQSIKGIMVEPGFGTWIKRQSIQKLDKTIKF

SEQ ID NO: 141 - protein, 497 aa, Bacillus terrae
  4-ureido-5-carboxyl-imidazole-amide hydrolase
MDLILKNALIPQGDRKVKTNILVDNGKIVGFISALNGLSADRVIDMKENLVVPGCIDPHTHFMDPGFTHRETFTTGTRSAAAGGVTTHIDMPCCSKPSVRDAESFHKKLAPLKDQAYIDFAFWGGMTGEDVREGLLDNIQPQIDEGVVAFKVYMTPSVPTFPKVSDAEMLEIFSHIAPTGMPIGVHAENYDICTFYSEKLKKEGRLDGPAWAEARMALAEKAAIQLIISFAEATGARVHVVHMSTKEGVELVREAKKRGVKVTAETCPHYLMLNAEDSMSERGTFAKIAPPLREKEDNIVHWQALADGTIDFVGTDHAPYEIPTEKDAQDSTIWNSFPGIPGVETMVPILVSEGLNKGRLSLSRFVEVTSRNAAIHFGIYPKKGAMEIGSDADFTVIDLEREYTIDEQKTESMAKYNPLHGLKLKGKPIQTIVRGQVVYDEDNGGITGAAGYGQFVNRQSIQSLERVIKYETYEERAKEAEASPRIEKALRRDLITN

SEQ ID NO: 142 - protein, 482 aa, Tindallia californiensis
  4-ureido-5-carboxyl-imidazole-amide hydrolase
MKVDVVVTNARIPQGDDTVLTNILVKDEKIAGFVSELDGVTYDREIDAKGHLTLPGCFDSHTHFMDPGFEHRENFLTGTSSAAAGGLTTIMDMPCCSKPSVRSIPELESKLNRIKDKAIIDFAFWGGVTGEDIRNGDYHVVQEQADYGVCAFKVYMTPSVPTYERVTDPEMLEAFRLVAKTGLVVGIHAENFAMCDFYVKKFQAEGRTDGPAWAEARLELAEKAAIQLGISFAEETGARLHIVHMSTGIGAKLVGAAKQKGLDVTSEACPHYLMLNYQDSMTEHRQFAKIAPPLRTKKDNEELWEGLQNGSVDFMGTDHAPYEIETEKEGIVDGVALDIWNSFPGIPGVETLVPIMVSEGYLKGRLSLSRLVEVLSTNAAKQYGIYPKKGALHIGSDADFTIIDLEKEWTIDQKETYSMAKYNPLHGIKLKGKPYKTVVRGNTVYEDGKGIVGKPGYGQFVKRQNNQKLSRTIHFTGNDFRP

SEQ ID NO: 143 - protein, 555 aa, Clostridium acidurici
  4-ureido-5-carboxyl-imidazole-amide hydrolase
MSYDVIIKNAKIPQGDNTVLTNILVKDEKIAGFVSTIHGIEAKETIDAEGHLTFPGFIDSHTHIMYHGFGHRENWVTGTSAAARGGVSTIIDMPCCTVPSTRTVKQLELKIDIANAQTIVDYGLWGGVTGEDVREGWMHNVKEQADFGVVAFKVYMTPSVPTYPRVTDPEMLECFKEVAKTGLPIGIHAENFAICDFYVKKFQEEGRMDGPAWSEARNELAEKVAIELGISFAEATGARLHIVHMSTGIGAKLIGEAKKRGINATSESCPHYLTLNYQEAMTKHGSQAKIAPPIRTSADNEEQWEGLRNGSIDFMATDHAPYEIESEKNKEGMNIWTAFPGIPGVETMAPIIISEGLNKGRLSLSRTVEVLSTNAAKHYGLYPKKGAMEIGSDADFAIVDLEKKWTIDAKGNQASMCGYTPLDGMELKGRVLKTIVRGTVVFDDTQEGVLPTLTDAELEKTVHNFPEGTEEKFADIFEQFPELAAAEYDRSYRKLHPEKINEDVRRIKGIMVKPGFGKYVKRQSIQVLPKTITYGTTTPTGVNRRERESLPEFNK

SEQ ID NO: 144 - protein, 515 aa, Romboutsia lituseburensis
  4-ureido-5-carboxyl-imidazole-amide hydrolase
MVDVIIKNARIPQGDNTIVTNILVKNEKIAGFVDDISMIDAKEVIDANENLTIPGCIDSHTHFMYQGFPHRENFLTGTAAAATGGVTTIIDMPCCSVPSVRSIEQLETKLSLCGPQAVVDYAMWGGVTGEDVREGWMHNVKEQADYGVVAFKVYMTPSVPTYPRVTDPEMYECFKAVSKTGLPIGIHAENFAMCDFYVNKFKNEGRLDGPAWAEARMELAEKVAIELGISFAEASGARLHIVHMSTGIGAKLVKDAKMRGLDITSESCPHYLTLNYQESMSKYGPLAKIAPPLRTKKDNEEHWQGINDGSIDFIATDHAPYEIESEKHKEGMNIWTSFPGIPGVETMVPVVVSEGYNKGRISLSKLVDILSTNAAKHYGLYPKKGALNIGSDADFTIIDLEKEWTIDPKTMVSMCGYTPLEGMKLKGKVFKTVVRGNLVYQDGENGTLGSLDNYDVVFNNPEELTSGEYKVSYRKEHPEVILNHVKNIKGIMVKPGFGKFTKRQQIMKLDKKIKF

SEQ ID NO: 145 - protein, 514 aa, Paraclostridium bifermentans
  4-ureido-5-carboxyl-imidazole-amide hydrolase
MDLIIKNANIPQGDEMVLTNILVKDEKIVGFVDDINMIDAKEIIDAKENLVMPGCIDSHTHFMYQGFPHRENFLTGTAAAATGGVTTIIDMPCCSVPSVRSTEQLETKLGICGPQAVVDYAMWGGVTGEDVREGWMHNVKEQADYGVVAFKVYMTPSVPTYPRVTDPEMFECFKAVSETGLPIGIHAENFAMCDFYVNKFKEEGRLDGPAWAEARMELAEKVAIELGISFAEASKARLHIVHMSTGVGAKLVKEAKMRGLDVTSESCPHYLTLNYQDAMTEYGPLAKIAPPLRTKKDNEEHWKGINDGSIDFIATDHAPYEIESEKLKEGTNIWTSFPGIPGVETMVPVIVSEGYNKGRISLSKLVDILSTNAAKHYGLYPKKGALNIGSDADFAIIDLNKEWTIKNENLVTMCGYTPLEGMKLKGRIVKTIVRGNLVYEDGEAGVLGNLENYDVLFNNPEELTSGEYKVSYRKDHPELILEHVKNIKGIKVKPGFGKFIKRQTISKLDKNIKF

SEQ ID NO: 146 - protein, 486 aa, Bacillus praedii
  4-ureido-5-carboxyl-imidazole-amide hydrolase
MDLILKNANIPQGDRQVLTNILVNEGKIVGYSNDISFLNAEKVIDIGGKLVVPGCIDPHTHFMDPGFTHRETFATGSRSAIAGGLTTIIDMPCCSKPSVRDGESFQKKIGPIRDQAYVDYCFWGGMTGEDAREGWLDNVYPQIEEGVVAFKVYMTPSVPTFPRVSDAEMLEIFKAVSKTGLPIGVHAENYDICTFNSKKLEKEGRFDGPAWAEARSTLAEKVAIELIISFAEATDARVHVVHMTTKEGAELVKAAKKRGVKVTAETCPHYLVLNAKDSMSERGSFAKIAPPLRGKEDNEAHWQALADGTIDFVGTDHAPYEIATEKDFPGATIWNTFPGIPGVETMVPILVSEGLNKGRLSLSRFVEVTSRNAAIHYGIYPKKGSMEIGSDADFTVIDLEKEYEIDEQKTESMAKYNPLHGMKLKGKPVQTIIRGTVVYEDDKGVVGQAGYGQYVPRQSIQRLDRTFKYEKFEAAKVEAKETVKQN

SEQ ID NO: 147 - protein, 471 aa, Carbydothermus islandicus
  4-ureido-5-carboxyl-imidazole-amide hydrolase
MGFDLVLKNAKIPQGNRTVDTHILVKDGKIAGFTNDLTGIEVAEVIDAENNLVLPGCIDSHTHFNDPGFTHRENFATGTQAAAAGGVTTIIDMPCCSVPSVRSVDNLEAKLEVIKDKAYVDYAMWGGATGEDVRNNALYNIKEQADYGVVAFKVYMTPSVPTYPPASDAEMLEIFREVAKTGLPVGIHAENLDLCTYHVNKLRKEGRFDGPAWAEARLELAEKVAIQMAISFAEETGARLHIVHMSTGIGGVLVGEAKKRGIDVTAETCPHYLVLNAYESMTKWRQFAKIAPPLRTTKDNEMLWEQLADGRVDFIATDHAPYEIATEKEAEGMDIWTAFPGIPGVETMVPIIVSEGYNKGRLSLSRLVDVLSRNAAIHYGLYPKKGALEIGSDADFTVIDLDYEWTIDKDKMYTMAKYTPLHGMKLKGKPVKTIVRGKLVYDDKNGIVGKPGYGQFVKRQTISKLERWLKF

SEQ ID NO: 148 - protein, 471 aa, Caloramator australicus
  4-ureido-5-carboxyl-imidazole-amide hydrolase
MRYDVIVKNARIPKGDDTVLTNILVKDEKIAGFIDNLEGIEADTIIDAEGHLTLPGCIDSHTHYMDPGFTHRENFLTGTSGAAVGGVTTIIDMPCCSVPSVRGVAELHNKINAIKDKAIVDYAMWGGVTGEDVRNKKLNIVEEQAAEGVVAFKIYMTPSVPTYPRVTDPEMLEIFRAVAKTGLPVGVHAENFAICDYMVKKFQEEGRMDGPAWAEARMELAEKVAIQLCISYAEETGARFHVVHMSTGIGAKLVKEAKMKGLDVTSETCPHYLTLNYQEHMTKFKQFVKIAPPIRTKKDNEELWQGLQDGSVDFIATDHAPYEIPTEKDAPGMTIWTAFPGIPGTETMVPIIVSEGYNKGRISLSRLVEILSTNAAKHYGLYPKKGTLMIGSDADFTIIDLNKEWVIEKEKMQCLNKYTPFDGMKLKGKVVKTIVRGTLVYDDEKGIVGKPGYGQWIKRQSIQKLDRVIKF

SEQ ID NO: 149 - protein, 514 aa, Paraclostridium bifermentans
  4-ureido-5-carboxyl-imidazole-amide hydrolase
MDLIIKNANIPQGDEMVLTNILVKDEKIVGFVDDINMIDAKEIIDANKNLVMPGCIDSHTHFMYQGFPHRENFLTGTAAAATGGVTTIIDMPCCSVPSVRSIEQLETKLGICGPQAVVDYAMWGGVTGEDVREGWMHNVKEQADYGVVAFKVYMTPSVPTYPRVTDPEMFECFKAVSETGLPIGIHAENFAMCDFYVNKFKEEGRLDGPAWAEARMELAEKVAIELGISFAEASKARLHIVHMSTGIGAKLVKEAKMRGLDVTSESCPHYLTLNYQDAMTEYGPLAKIAPPLRTKKDNEEHWKGINDGSIDFIATDHAPYEIESEKLKEGTNIWTSFPGIPGVETMVPVIVSEGYNKGKISLSKLVDILSTNAAKHYGLYPKKGALNIGSDADFAIIDLNKEWTIKNENLVTMCGYTPLEGMKLKGRIIKTIVRGNLVYEDGEAGVLGKLENYDVVFNNPEELTSGEYKVSYRKDHPELILEHVKNIKGIKVKPGFGKFIKRQTISKLDKNIKF

SEQ ID NO: 150 - protein, 533 aa, Tepidimicrobium xylanilyticum
  4-ureido-5-carboxyl-imidazole-amide hydrolase
MVYDVIIKNARIPQGDDTVITNILVKDEKIAGFLDNVVGIEAKEVIDANGHLTLPGCIDSHTHFMYQGFPHRENFLTGSAAAARGGITTVIDMPCCSVPSARSVEQLKLKIELVEPQALVDYAMWGGVTGEDVREGWLHNVKEQADFGAVAFKVYMTPSVPTYPRVSDPEMYEAFKAVAETGLPIGIHAENFAMCDYFVKKFQREGRMDGPAWAEARMELAEKVAIQLGISFAEDTGARLHIVHMSTGIGAKLVGEAKARGLNVTSETCPHYLTLNYQEAMTKYGPLAKIAPPLREKKDNEMLWEGLNNGSVDFIATDHAPYEIESEKHKKGMNIWTAFPGIPGVETMVPIIVSEGYNKGRISLSKMVDILSTNAAKHYGLYPKKGAMHIGSDADFTIIDLEKRWTIDLKDQASMCGYTPLEGMELKGKVVKTIVRGHLVFEDVEGSTLKDLSDEELQRIVHKYPEGVEERYSEIFKEFPNLHSQEYEKSYRKEHPELIDQHIRGIKGIKVRPGFGKFVKRQSIQVLPRTITY

SEQ ID NO: 151 - protein, 490 aa, Aneurinibacillus migulanus
  4-ureido-5-carboxyl-imidazole-amide hydrolase
MDLILKNANIPQGDRQVQTNILVENGTIVGFVNSINGLQAKNVIDLKGKLVVPGCIDPHTHFMDPGFTHRETFATGTMSAAAGGLTTIIDMPCCSKPSVRSEESFYAKIDPIKDKAYIDYCFWGGMTGEDVREGLLDNVWGQKEAGVVAFKSYMTPSVPTFPKVSDAELLEIFYKVAETGLPVGVHAENYDICSFYTEKLQKEGRLDGPAWAEARKALAEKVAIELIISYAEETGARAHIVHMSTKEGAHLIRQAKERGVKITAETCPHYLVLNAQESMTKYGTFAKIAPPLRGVEDNEIHWQALADGTIDFVGTDHAPYEIPTEKAAEDSDIWNSFPGIPGVETMVPILVSEGLNKGRLSLSRFVEVTSRNAAIHYGIYPKKGAIEIGSDADFTVIDLEKEHVIDQEQTYSMAKYNPLHDIKLKGMPVMTIVRGNVVYEDGKGIVGQEGYGQYVKRQSTQQLDAVINLTKSNESKAVSQSSEKKESVKL

SEQ ID NO: 152 - protein, 497 aa, Bacillus bacterium
  4-ureido-5-carboxyl-imidazole-amide hydrolase
MDLILKNALIPQGDRKVKTNILVDNGKIVGFTSALNGLSADRVIDIKENLVVPGCIDPHTHFMDPGFTHRETFTTGTRSAAAGGVTTHIDMPCCSKPSVRDAESFHKKLAPLKDQAYIDFAFWGGMTGEDVREGLLDNIHPQIDEGVVAFKVYMTPSVPTFPKVSDAEMLEIFSHIAPTGMPIGVHAENYDICTFYSEKLKKEGRLDGPAWAEARMALAEKTAIQLIISFAEATGARVHVVHMSTKEGVELVRAAKKRGVKVTAETCPHYLMLNAEDSMSERGTFAKIAPPLREKEDNIVHWQGLADGTIDFVGTDHAPYEIATEKDAPDSTIWNSFPGIPGVETMVPILVSEGLNKGRLSLSRFVEVTSRNAAIHFGIYPKKGALEIGSDADFTVIDLEREYTIDEQKTESMAKYNPLHGLQLKGKPIQTIVRGRVVYDEDNGGIVGEAGYGQYVKRQAIQSLERVIKYETYEEGAKEAEAKPRVEKALRRDLITN

SEQ ID NO: 153 - protein, 489 aa, Bacillus notoginsengisoli
  4-ureido-5-carboxyl-imidazole-amide hydrolase
MDLILKNANIPQGDRQVLTNVLVDEGKIVGFTNDINFLHADEIIDIKGKLVVPGCIDPHTHFMDPGFTHRETFETGHRSAAAGGLTLHIDMPCCSKPSVRDGESFFKKLNPIKEKAYIDYCFWGGMTGEDAREGWLDNIYPQIEQGVVAFKVYMTPSVPTFPKVSDGEMFEIFRKVAPTGLPIGVHAENYDICTFNTERLKNEGRTDGPAWAEARSSLAEKVAIELIISYAEEVDARVHVVHMSTKEGVELVRRAKERGLKVTAETCPHYLMLNAKDAMTERGTFAKIAPPLRGKADNEAHWQALADGTIDFVGTDHAPYEIPTEKAAPDSTIWNSFPGIPGVETMVPVLVSEGLNKGRLSLSRFVEVTSTNAAKHYGIYPKKGAMEIGSDADFTVIDLDREYEIDEQKTESMAKYNPLHGLQLKGKPVQTIVRGKLIYDEDNGGIVGEAGYGQFVPRQSIQRLERVLKIEKYEAAAVTEKSEKAEVIN

SEQ ID NO: 154 - protein, 486 aa, Bacillus oceanisediminis
  4-ureido-5-carboxyl-imidazole-amide hydrolase
MDLILKNASIPQGDRQVLTNILVNEGKIVGYTNDISFLNAARVIDIKGKLVVPGCIDPHTHFMDPGFTHRETFATGSRSAAAGGLTTIIDMPCCSKPSVRDGESFNKKIGPIRDQAYIDYCFWGGMTGEDAREGWIDNIYPQIDQGVVAFKVYMTPSVPTFPKVSDAEMLEIFSNVAKTGLPIGVHAENYDICTFYSKKLEKEGRLDGPAWAEARSSLAEKVAIELILSFAEATDARVHVVHMSTKEGVELVKAAKKRGVKVTAETCPHYLVLNAKDSMSERGSYAKIAPPLRGKEDNESHWQALADGTIDFVGTDHAPYEIATEKDYPGATIWNTFPGIPGVETMVPILVSEGLNKGRLSLSRFVEVTSRNAAIHYGIYPKKGSMEIGSDADFTVIDLEKEYVIDEQKTESMAKYNPLHGMKLKGKPVQTIIRGNVIFDEDKGIVGEAGYGEYVPRQSIQRLERTFKYEKYEAVKAKTKDPVAQQ

SEQ ID NO: 155 - protein, 471 aa, Paenibacillus sp.
  4-ureido-5-carboxyl-imidazole-amide hydrolase
MSYDLIIKNASIPQGDRQVLTDILVNKGVIVGFAKDIEADAAEIIDAGGKLVLPGVIDSHTHFNDPGFTHREDFVTGTSSAAAGGVTMIVDMPCCSVPSVRSADNLFNKLNALEGKGIVDYAMWGGITGEDVRNNVLPEIISEQIANGVVAFKVYMTPSVPSYPRVTDPEMLEAFAAVSPTGLPIGIHAENFAICDYFTNKLKGEGRLDYPAWAEARMALAEKAAIQLGISFAEETGARLHIVHMSTGAGNELIREAKLKGTKVTAESCPHYLTLNAVDAMTEFGTFAKIAPPLRTAKDNEVLWQGLADGTIDFMATDHAPYEIATEKDAPGMDVWTSFPGIPGVETLVPIMISEGYNKGRLSLSRLVELLCTNPAIHYGLYPKKGALEIGTDADFTIVDLEKEWTLDKDQMHCKPKYTPFHGMKLKGKVDKTIVRGTLVYDDALGVVGKPGHGEYVKRQTISELPRLLKY

SEQ ID NO: 156 - protein, 470 aa, [Clostridium] ultunense Esp
  4-ureido-5-carboxyl-imidazole-amide hydrolase
MYQVIVKNARIPQGDETVLTNILIKDERIAGFVDGIDGIEAEEIIDAEGHLTLPGCIDSHTHFMDPGFTHRENFLTGTSSAAAGGLTTIVDMPCCSVPSVRSIPELENKLEKIKDKAIIDYGFWGGVTGEDVREGWLHNVKEQADYGVIGFKVYMTPSVPTYPRVTDPEMYEAFRTVAETGLPIGIHAENFAMCDYFVKKFQKEGRMEGPAWAEARMELAEKVAIQLGISFAEDTGARLHIVHMSTGIGAKLVKEAKLKGLDVTSETCPHYLTLNYEEAMSEYGAFAKIAPPLRTKRDNKELWEGLRDGSVDFIATDHAPYHIEDEKAKKGMNIWTAFPGIPGVETMVPIIVSEGYNKGRISLSKMVEILSTNAAIHYGLYPKKGSLNIGADADLTIIDLDKEWAIDQEKTYSMAKYNPLHGLKLKGKVAKTVVRGKVVFDEKEGITVKEGYGQFVKRQSIQKLDKKIKF

SEQ ID NO: 157 - protein, 497 aa, Bacillus freudenreichii
  4-ureido-5-carboxyl-imidazole-amide hydrolase
MDLILKNALIPQGDRKVKTNILVDDGKIVGFTSALNGLSADRVIDMKENLVVPGCIDPHTHFMDPGFTHRETFTTGTRSAAAGGVTTHIDMPCCSKPSVRDAESFHKKLAPLKDQAYIDFAFWGGMTGEDVREGLLDNIQPQIDEGVVAFKVYMTPSVPTFPKVSDAEMLEIFSHIAPTGMPIGVHAENYDICTFYSEKLKKEGRLDGPAWAEARMALAEKAAIQLIISFAEATGARVHVVHMSTKEGVELVRAAKKRGVKVTAETCPHYLMLNAEDSMSERGTFAKIAPPLREKEDNIVHWQALADGTIDFVGTDHAPYEIPTEKDAPDSTIWNSFPGIPGVETMVPILVSEGLNKGRLSLSRFVEVTSRNAAIHFGIYPKKGAMEIGSDADFTVIDLEREYTIDEKKTESMAKYNPLHGLKLKGKPIQTIVRGRVVYDEDNGGIMGEAGYGQYVKRQSIQSLERVIKYETYEEGAKEAEAKPRMEKALKRDLITN

SEQ ID NO: 158 - protein, 471 aa, Caloramator fervidus
  4-ureido-5-carboxyl-imidazole-amide hydrolase
MRYDCIVKNARIPQGDDTVLTNILVKDEKIAGFVDNIEGIEADTIIDAEGHLTLPGCIDSHTHYMDPGFTHRENFLTGTKGAAVGGVTTIIDMPCCSVPSVRGVAELYNKINAIKDKAIVDYAMWGGVTGEDVRNKKLNIIEEQAAEGVVAFKIYMTPSVPSYPRVTDPEMLEIFKEVAKTGLPVGVHAENFAICDYMVKKFQEEGRMDGPAWAEARMELAEKVAIQLCISYAEETGARFHVVHMSTGIGAKLVKEAKMKGLDVTAETCPHYLTLNYQEHMTKYKQFVKIAPPIRTKKDNEELWKGLQDGSVDFIATDHAPYEIATEKDAPGMNIWTAFPGIPGTETMVPIMVSEGYNKGRISLSRLVEILSTNPAKHYGLYPKKGALMIGSDADFTIIDLNKEWIIKKEEIQCLNKYTPFDGMKLKGKVVKTIVRGRLVYDEEKGIVGEPGYGKWIKRQSIQKLDRVLKF

SEQ ID NO: 159 - protein, 486 aa, Bacillus oceanisediminis
  4-ureido-5-carboxyl-imidazole-amide hydrolase
MDLILKNANIPQGDRQVLTNILVNKGKIVGYTNDISFLNAARVIDIKGKLVVPGCIDPHTHFMDPGFTHRETFATGSRSAAAGGLTTIIDMPCCSKPSVRDGESFNKKIGPIRDQAYIDYCFWGGMTGEDAREGWIDNIYPQIDQGVVAFKVYMTPSVPTFPKVSDAEMLEIFSNVAKTGLPIGVHAENYDICTFYSKKLEKEGRLDGPAWAEARSSLAEKVAIELILSFAEATDARVHVVHMSTKEGVELVKAAKKRGVKVTAETCPHYLVLNAKDSMSERGSYAKIAPPLRGKEDNESHWQALADGTIDFVGTDHAPYEIATEKDYPGATIWNTFPGIPGVETMVPILVSEGLNKGRLSLSRFVEVTSRNAAIHYGIYPKKGSMEIGSDADFTVIDLEKEYVIDEQKTESMAKYNPLHGMKLKGKPVQTIIRGNVIYDEDKGIVGEAGYGEYVPRQSIQRLERTLKYEKYEAVKAKTKDPVAQQ

SEQ ID NO: 160 - protein, 533 aa, Soehngenia saccharolytica
  4-ureido-5-carboxyl-imidazole-amide hydrolase
MMYDVIIKNARIPQADETTLTNILVKDEKIAGFIDNIDGIDAKEIIDANGHLTLPGCIDSHTHFMYQGFPHRENFLTGTAAAAAGGVTTIIDMPCCSVPSVRSVEQLKLKLDAVSPQALVDFAFWGGVTGEDVRNGWLHNVEEQAEYGVVAYKAYMTPSVPTYPRVDDPEMLEAFTEVAKTGLPIGIHAENYAMCDYYVKKYQREGRTDGPAWAEARLELAEKVAIELGISFAEHTGARLHIVHMSTGIGAKLIGEGKKRGLNVTSETCPHYLTLNYQEAMTKYGPLAKIAPPLRTKKDNEELWEGLRNGSVDFMATDHAPYELESEKLSKGMNIWTSFPGIPGVETMVPVLISEGYNKGRLSLSKLVEVLSTNAAKHYGIYPKKGAMNIGSDADFTIIDLDKRWKVDPKKLASMCGYTPLEGMELKGKVAKTVLRGHLIYEDNDESELAKMTDEDMQRIVHNYPEGLEERYKDILKEFPHLHSKEYENSYRKQHPEIIDEHIRGIKGIKVKPGFGQFVKRQSIQVLPRRITF

SEQ ID NO: 161 - protein, 486 aa, Bacillus firmus
  4-ureido-5-carboxyl-imidazole-amide hydrolase
MDLILKNANIPQGDRQVLTNILVNEGKIVGYTNDISFLNAAKVIDIQGKLVVPGCIDPHTHFMDPGFTHRETFATGSRSAAAGGLTTIIDMPCCSKPSVRDGDSFNKKIGPIRDQAYIDYCFWGGMTGEDAREGWIDNIYPQIDQGVVAFKVYMTPSVPTFPKVSDAEMLEIFKNVAKTGLPIGVHAENYDICTFNSKKLEKEGRLDGPAWAEARSSLAEKVAIELILSFAEATDARVHVVHMSTKEGVQLVKAAKKRGVKVTAETCPHYLVLNAQDSMSERGSYAKIAPPLRGKEDNESHWQALADGTIDFVGTDHAPYEIATEKDYPGATIWNTFPGIPGVETMVPILVSEGLNKGRISLSRFVEVTSRNAAIHYGIYPKKGSMEIGSDADFTVIDLEKEYVIDEQKTESMAKYNPLHGMKLKGKPVQTIIRGNVVFEEDKGIVGEAGYGEYVPRQSIQRLERTLKYEKYEAVNVKTKDPVAQQ

SEQ ID NO: 162 - protein, 471 aa, Thermotalea metallivorans
  4-ureido-5-carboxyl-imidazole-amide hydrolase
MQVDVMIKNARIPQGNDTILTNILIVDEKITGFVPNTEGIEAASVIDAEGHLTIPGCIDSHTHINDPGFTHRENFLTGTSAAASGGVTTIIDMPCCSVPSVRSIENLEHKLNAIKDKAIIDYAMWGGVTGEDVREGWLHHVKEQADYGVVAFKVYMTPSVPTYPRVTDPEMLEAFRAVAETGLPIGIHAENFAMCDYYVKKFQNEGRMDGPAWAEARSHLAEKVAIELGISFAEETGARLHIVHMSTGIGAKLVGEAKKRGVDVTAETCPHYLTLNYKDAMTEFKQFAKIAPPLRCKEDNEIHWEALRDGRIDFVATDHAPYEIPTEKEAEGMNIWTAFPGIPGVETMVPVIVSEGYNKGRLSLSRMVEVLSTAPAKHYGIYPKKGAMHIGSDADFTIIDLEKEWVIDKDKMYTMAKYTPFHGFKLKGKPVKTVVRGKLVYDDKVGIVGREGHGQFIRRQSIQKLDRILKF

SEQ ID NO: 163 - protein, 493 aa, Ornithinibacillus halophilus
  4-ureido-5-carboxyl-imidazole-amide hydrolase
MDLILKNALIPQGDRKVQTNILVDNGKITGYTNSVDGLYADEILDMKGNLVVPGCIDPHTHFMDPGFTHRETFTTGSRSAAAGGVTTHIDMPCCSKPSVRDGDSFHKKITPLKGQAYIDYCFWGGMTGEDVRNGWLNNIYPQIEEGVVAFKVYMTPSVPTFPKVTDAEMLEIFSHIAPTGMPIGVHAENYDICTFYSEKLKKEGRMDGPAWAEARMPLAEKVAIQLILSYAEATDARVHVVHMSTKEGVELVRDAKKRGVKVTAETCPHYLMLNAQDSMTERGTFAKIAPPLRERMDNIVHWQALADGTIDFVGTDHAPYEIETEKDAPDSDIWNSFPGIPGVETMVPILVSEGLNKGRLSLSRFVDVTSRNAAIHYGIYPKKGSMELGADADFTVIDLDREYMIDEKQTESMAKYNPLHGLHLKGKPIQTIVRGKVVYDEDNGGIVGEAGFGEYVKRQSIQRLDREIKYEVYEEKAKELESTKRIEHALVQK

SEQ ID NO: 164 - protein, 475 aa, Thermosyntropha lipolytica
  4-ureido-5-carboxyl-imidazole-amide hydrolase
MKFDTVVKNAMIPQGDRLVPATILVKDGVIAGIVNGEMEPHLLEGVNVIDAEGNMVLPGCIDSHTHFMDPGFTHRETFLDGCKAAAAGGLTTIIDMPCCSVPSVRGVAEMENKLNAIKPQAIIDFGLWGGVTGEDVRNEVHYKIVREQADAGVVAFKVYMTPSVPSYPRVTDPEMLECFKAVAETGLPIGIHAENYAMCDFYVQKFQKEGRMDGPAWSEARMPLAEKVAIQLGISFAEATGARLHIVHMALGEGAILVGEAKKRGLDVTSETCPHYLVLDYKEAMSKYGAFAKIAPPLRSREDNELLWQGLQNGSVDFIATDHAPYEIPTEKAAEDMTIWTAFPGIPGVETMVPIIISEGYNKGRIKLSRLVEILSRNAAIHYGLYPKKGAMEIGSDADFTIIDLNKEWVIDQEKQYSKAKYTPLHGMKLKGKVVKTVVRGKVVYDEAKGVIGEPGYGEFVKRQTISKLDRKITF

SEQ ID NO: 165 - protein, 490 aa, Fictibacillus sp.
  4-ureido-5-carboxyl-imidazole-amide hydrolase
MDLILKNAMIPQGDRTVITNILVQEGRITGFCNDIQTLNADKVLDMEGKLVVPGCIDPHTHFMDPGFTHRETFATGSRSAAAGGLTTIIDMPCCSKPSVRDGESFQKKIAPIRGQAYIDYCFWGGMTGEDARNGWIDNIYPQIDEGVVAFKVYMTPSVPTFPKVSDGEMLEIFQKVAKTGMPIGVHAENYDICTFYTEKLKNEGREDGPAWAEARMALAEKVAIELIISYAEATDARVHVVHMSTKEGVKLVREAKKRGVKVTSETCPHYLMLNAQDSMTERGTFAKIAPPLRGKEDNQAHWQALADGTIDFVGTDHAPYEIATEKAAADSSIWNSFPGIPGVETMVPILVSEGLNKGRLSLSRFVEVTSKNAAVHYGIYPKKGAMEIGSDADFTVIDLDHEWVIDEKQTESMAKYNPLHGIKLKGRPVQTILRGTVVYDYEKGGITGTAGYGEYVKRQSIQRLERMLKYENYETANQPSAARQQLEKSK

SEQ ID NO: 166 - protein, 489 aa, Bacillus mesonae
  4-ureido-5-carboxyl-imidazole-amide hydrolase
MDLILKNASIPQGDRQVLTNILVDEGKIVGFTNDINFLHADKMIDIEGKLVVPGCIDPHTHFMDPGFTHRETFETGHRSAAAGGLTLHIDMPCCSVPSVRDGESFFKKLNPIKEKAYIDYCFWGGMTGEDAREGWLDNIYPQIEQGVVAFKVYMTPSVPTFPKVSDGEMFEIFRKIAPTGLPIGVHAENYDICTFNTERLKKEGRQDGPAWAEARSSLAEKVAIELIISYAEAVDARVHVVHMSTKEGVELVRRAKERGIKVTAETCPHYLMLNAKDSMTERGTFAKIAPPLRGKEDNEAHWQALADGTIDFVGTDHAPYEIATEKDAPDSDIWNSFPGIPGVETMVPILVSEGLNKGRLSLSRFVEVTSTNAAKHYGIYPKKGALEIGSDADFTVIDLNREYEIDEQQTESMAKYNPLHGLKLKGKPVQTIVRGNLIYDEDHGGIVGEAGYGEYVPRQSIQRLERVLKYEKYEASVVTKKSEEAEIAK

SEQ ID NO: 167 - protein, 531 aa, Clostridium purinilytica
  4-ureido-5-carboxyl-imidazole-amide hydrolase
MYDLILKNARIPLGDNTIVTNILVKDEKISGISNCIEGVEAKETIDVEEQLTLPGCIDSHTHFMYQGFPHRENFLTGTAAAATGGITTIIDMPCCTVPSARSVDQLKLKIDLCQPQSVVDFAMWGGVTGEDVREGWMHNVKEQADYGVVAFKVYMTPSVPTYPRVTDPEMLECFRAVAETGLPIGVHAENFAMCDFYVKKFQKGGKLDATAWSEARMELAEKVAIELGISFAEETNARLHIVHMSTGIGAKLIGEAKKRGINATSETCPHYLTLNYQDALKEHGAFAKVAPPLRNKKDNEELWEGLKNGSVDFIATDHAPYEIESEKNREDMTIWTAFPGFPGVETMVPILISEGYNKGRVSLSKLVDILSTNAAKHYGLYPKKGTIAIGSDADFTIIDLNKEWTITKETVTMCGYTPFKGMKLKGKPVKTIVRGHVVYDEKQEGTLPQLTDEELKTIVHEYPSGVEEKYSEIFEKFPHLHSDEYKRSYRKDHPELIDKHIRGIKGIMVKPGFGQFVKRQSIQILPKTITY

SEQ ID NO: 168 - protein, 482 aa, Tindallia magadiensis
  4-ureido-5-carboxyl-imidazole-amide hydrolase
MKVDVVVTNARIPQGDDTVLTNILVKDEKIAGFVSELEGVTYDREIDAKGHLTLPGCFDSHTHFMDPGFEHRENFLTGTSSAAAGGLTTIMDMPCCSKPSVRSIPELESKLNRIKDKAIIDFAFWGGVTGEDIRNGDYHVVQEQADYGVCAFKVYMTPSVPTYERVTDPEMLEAFRLVAKTGLVVGIHAENFAMCDFYVKKFQAEGRTDGPAWAEARLELAEKAAIQLGISFAEETGARLHIVHMSTGIGAKLVGAAKQKGLDVTSEACPHYLMLNYQDSMTEHRQFAKIAPPLRTKKDNEELWEGLQNGSVDFMGTDHAPYEIETEKEGVVDGVALDIWNSFPGIPGVETLVPIMVSEGYLKGRLSLSRLVEVLSTNAAKQYGIYPKKGALHIGSDADFTIIDLEKEWTIDQKETYSMAKYNPLHGIKLKGKPYKTVVRGNTVYEDGKGIVGKPGYGQFVKRQNIQKLSRTIHFSGNDFRP

SEQ ID NO: 169 - protein, 470 aa, Paenibacillus borealis
  4-ureido-5-carboxyl-imidazole-amide hydrolase
MGYELIIKNASIPQGDRQVLTDILVNKGVIVGFAKDIDAEADAIIDAQGKLVLPGVIDSHTHFNDPGFTHREDFVTGTSSAAAGGVTMIVDMPCCSVPSVRSVENLHNKLNALEGKGIVDYAMWGGITGEDVRNDVLNIVKEQAAEGVVAFKVYMTPSVPSYPRVTDPEMLEAFYAVAETGMPIGIHAENYAICDYYTQKLKKEGRMDYPAWAEARKILAEKAAIQLGISFSEESGARLHIVHMSTAAGNQLIKEAKLKGVKVTAESCPHYLTLNAVDAMTEFGTFAKIAPPLRRPEDNEALWQGLVDGTIDFMATDHAPYEIATEKDAPGMDVWTSFPGIPGVETLVPIMISEGYNKGRLSLSRLVELLCTNPAIHYGLYPKKGALEIGTDADFTIVDLEKEWTLDKDKMHCKPKYTPFHGMKLKGKVDKTIVRGTLVYDDELGVVGKPGYGEFVKRQTISELPRLLKY

SEQ ID NO: 170 - protein, 514 aa, Paraclostridium benzoelyticum
  4-ureido-5-carboxyl-imidazole-amide hydrolase
MDLIIKNANIPQGDEMVLTNILVKDEKIVGFVDDINMIDAKEIIDANKNLVMPGCIDSHTHFMYQGFPHRENFLTGTAAAATGGVTTIIDMPCCSVPSVRSVEQLETKLGICGPQAVVDYAMWGGVTGEDVREGWMNNVKEQADYGVVAFKVYMTPSVPTYPRVTDPEMFECFKAVSETGLPIGIHAENFAMCDFYVNKFKEEGRLDGPAWAEARMELAEKVAIELGISFAEASKARLHIVHMSTGVGAKLVKEAKMRGLDVTSESCPHYLTLNYQDAMTEYGPLAKIAPPLRTKKDNVEHWKGINDGSIDFIATDHAPYEIESEKLKEGTNIWTSFPGIPGVETMVPVIVSEGYNKGRISLSKLVDILSTNAAKHYGLYPKKGALNIGSDADFAIIDLNKEWTIKNENLVTMCGYTPLEGMKLKGRIIKTIVRGNLVYEDGEAGVLGNLENYDVLFNNPEELTSGEYKVSYRKDHPELIVEHVKNIKGIKVKPGFGKFIKRQTISKLDKNIKF

SEQ ID NO: 171 - protein, 486 aa, Bacillus solani
  4-ureido-5-carboxyl-imidazole-amide hydrolase
MELILKNANIPQGDRQVLTNILINDGKIVGFSNDISFLNAEKVIDIGGKLVVPGCIDPHTHFMDPGFTHRETFATGSRSAVAGGLTTIIDMPCCSKPSVRDGESFQKKIGPIRDQAYVDYCFWGGMTGEDAREGWLDNVYPQIDEGVVAFKVYMTPSVPTFPRVSDAEMLEIFKAVSKTGLPIGVHAENYDICTFNSKKLEKEGRFDGPAWAEARSTLAEKVAIELIISFAEATDARVHVVHMTTKEGAELVKAAKKRGVKVTAETCPHYLVLNAKDSMSERGSFAKIAPPLRGKEDNEAHWQALADGTIDFVGTDHAPYEIATEKDFPGATIWNTFPGIPGVETMVPILVSEGLNKGRLSLSRFVEVTSRNAAIHYGIYPKKGSMEIGSDADFTVIDLEKEYVIDEQKTESMAKYNPLHGMKLKGKPVQTIIRGNVVYEDGKGVLGQAGYGQYVPRQSIQRLERTFKYEKFEAVKVEAKETVKQN

SEQ ID NO: 172 - protein, 470 aa, Thermohalobacter berrensis
  4-ureido-5-carboxyl-imidazole-amide hydrolase
MYDLILKNARIPQGDDTVVTNILVKDGKISGFTQELDGIEAKEVMDVEGHLTLPGCIDSHVHINDPGFTHRENFLTGTSAAASGGITTVIDMPCCSVPSVRSVEQLENKLNAIKDKAIVDYAMWGGVTGEDVREGWLHNVKEQADYGVVAFKVYMTPSVPTYPRVTDPEMLEAFKAVAETGLPIGIHAENFDMCDFYVKKFEEEGRMDGPAWAEARMPLAEKVAIELGISFAEATGARLHIVHMSTGIGAKLVGEAKKRGLDVTAETCPHYLTLNYKDAMSKFKQFAKIAPPLRTEKDNEELWEGLQNGSVDFIATDHAPYEIATEKDAEGMNIWTAFPGIPGVETMVPIIISEGYNKGRLSLSRLVEVLSTSPAKHYGLYPKKGAMFIGSDADFTVIDLDKEWTIDKDKMYTKSKYTPFHGYKLKGKPVKTIVRGKLVYDDENGIVGQEGYGEFIKRQSIQKLDKAIKY

SEQ ID NO: 173 - protein, 514 aa, Acinetobacter sp. RIT592
  4-ureido-5-carboxyl-imidazole-amide hydrolase
MDLIIKNANIPQGDEMVLTNILVKDEKIVGFVDDINMIDAKEIIDANKNLVMPGCIDSHTHFMYQGFPHRENFLTGTAAAATGGVTTIIDMPCCSVPSVRSIEQLETKLGICGPQAVVDYAMWGGVTGEDVREGWMHNVKEQADYGVVAFKVYMTPSVPTYPRVTDPEMFECFKAVSETGLPIGIHAENFAMCDFYVNKFKEEGRLDGPAWAEARMELAEKVAIELGISFAEASKARLHIVHMSTGVGAKLVKEAKMRGLDVTSESCPHYLTLNYQDAMTEYGPLAKIAPPLRTKKDNAEHWKGINDGSIDFIATDHAPYEIESEKLKEGTNIWTSFPGIPGVETMVPVIVSEGYNKGRISLSKLVDILSTNAAKHYGLYPKKGALNIGSDADFAIIDLNKEWTIKNENLVTMCGYTPLEGMKLKGRIIKTIVRGNLVYEDGEDGVLGKLENYDVLFNNPEELTSGEYKVSYRKDHPELIVEHVKNIKGIKVKPGFGKFIKRQTISKLDKNIKF

SEQ ID NO: 174 - protein, 471 aa, Maledivibacter halophilus
  4-ureido-5-carboxyl-imidazole-amide hydrolase
MTYDVIVKNARIPQGDDTVVTNILVKDQKIAGFTMDLDGIQASKFIDAEGHLTLPGCIDSHTHINDPGFTHRENFLTGSSAAASGGVTTIVDMPCCSVPSVRSVENLEHKLNAIRDKAIIDYGMWGGVTGEDVRNGWLHNVKEQADYGVLAFKVYMTPSVPTYPRVTDPEMLEAFRAVAETGVPVGIHAENFALCDFYVKKYEKEGRMDPPAWAQARSTLAEKVAIELGISFAEETGARLHIVHMSSGIGAKLVGDAKKRGLDVTAETCPHYLTLNYQDAMTEFKQFAKIAPPLRTKEDNEEQWKALQDGRIDFVATDHAPYEIPTEKDAEGMHIWNAFPGIPGVETMVPVIVSEGYNKGRLSLSRLVEVLCSRPAKHYGIYPKKGAMFIGSDADFTIIDLDKEWTIDKDKMYTMAKYTPFHGYKLKGKPVKTILRGKLVYDDENGIVGEEGYGEFVRRQNIQKLDKTIKY

SEQ ID NO: 175 - protein, 531 aa, Tissierella creatinini
  4-ureido-5-carboxyl-imidazole-amide hydrolase
MYDVIIKNAKIPQGDNTILTNILVKDEKIAGFIDNIDNIDSKELIDAKGHLTLPGCIDSHTHFMYQGFPHRENFLTGTAAAARGGVTTVIDMPCCSVPSVRSVKQLQLKVDLIQPQALVDFALWGGVTGEDVREGWLDNVQEQADFGVVSFKAYMTPSVPTYPRVTDPEMYEAFKAVAKTGLPVGIHAENYAMCDYYVKKLREEGRTDGPAWEEARLELAEQVAIELGISFAEAADVRLHIVHMALGKGAQLVKAAKQRGLRVTSETTPHYLTINAKDAMTEHGAYAKIAPPLRSKDSNDLLWEGLRNGSVDFVATDHAPYEIESEKDAPGMNIWTAFPGIPGVETMVPIVVDGYNKGKISLSTMVNVLSKNAAIHYGLYPKKGAMEIGSDADFTIVDLDKEWTIDVKDQASMCGYTPLEGMKLKGRVDKTIVRGHLVFVDEGESSLANLTDEELQKVVHNFPEGVTEKYADEFAKYPHLNSAEYKNSYRINHPEVIDKHIRGIKGIVAKPGFGQWVKRQSIQVLPREIRY

SEQ ID NO: 176 - protein, 420 aa, Unknown
  compost metagenome 4-ureido-5-carboxyl- imidazole-amide hydrolase
MLPGVIDSHTHFNDPGFTHREDFVTGTSSAAAGGVTMIVDMPCCSVPSVRSADNLFNKLNALEGKGIVDYAMWGGITGEDVRNNVLPEIISEQIANGVVAFKVYMTPSVPSYPRVTDPEMLEAFAAVSPTGLPIGIHAENFAICDYFTNKLKGEGRMDYPAWAEARMALAEKAAIQLGISFAEETGARLHIVHMSTGAGNELIREAKLKGTKVTAESCPHYLTLNAVDAMTEFGTFAKIAPPLRTAKDNEVLWQGLADGTIDFMATDHAPYEIATEKDAPGMDVWTSFPGIPGVETLVPIMISEGYNKGRLSLSRLVELLCTNPAIHYGLYPKKGALEIGTDADFTIVDLEKEWTLDKDQMHCKPKYTPFHGMKLKGKVDKTIVRGTLVYDDALGVVGKPGHGEYVKRQTISELPRLLKY

SEQ ID NO: 177 - protein, 471 aa, Natronincola peptidivorans
  4-ureido-5-carboxyl-imidazole-amide hydrolase
MKYDVVVKNARIPQGDDTMLTHILVKDEKIAGFLTDLEGIQYEKVIDAEGHLTIPGCFDSHTHFMDPGFTHRENFLTGTSSAAAGGLTTIMDMPCCSKPSVRGVEELESKLNAIKDKAIIDYAMWGGVTGEDIRAGKKEIIKEQADYGVCAYKVYMTPSVPTYERVTDPEMLEAFRWVAETGLPVGIHAENYAMCDYYVNQFQKEGRMDGPAWAEARLELAEKAAIQLGISFAEETGARLHIVHMSTGIGAKLVGEAKKKGLDVTSETCPHYLMLNYQDAMTEHQQYAKIAPPLRTKKDNVELWEGLQNGSVDFMGTDHAPYEIATEKEAEGVNIWTAFPGIPGVETLVPIIVSEGYNKGRLTLSKMVEVLSTNAAKQYGLYPKKGAMHIGSDADFTIIDLEKEWTIDQKKTYSMAKYNPLHGIKLKGKPVKTVVRGTLVYDDEIGIVGKEGFGQFVRRQNIQKLDRVIKY

SEQ ID NO: 178 - protein, 486 aa, Bacillus firmus
  4-ureido-5-carboxyl-imidazole-amide hydrolase
MDLILKNANIPQGDRQVLTNILVNEGKIVGYTNDISFLNAAKVIDIQGKLVVPGCIDPHTHFMDPGFTHRETFATGSRSAAAGGLTTIIDMPCCSKPSVRDGDSFNKKIGPIRDQAYIDYCFWGGMTGEDAREGWIDNIYPQIDQGVVAFKVYMTPSVPTFPKVSDAEMLEIFKNVAKTGLPIGVHAENYDICTFNSKKLEKEGRLDGPAWAEARSSLAEKVAIELILSFAEATDARVHVVHMSTKEGVELVKAAKKRGVKVTAETCPHYLVLNAQDSMSERGSYAKIAPPLRGKEDNESHWQALADGTIDFVGTDHAPYEIATEKDYPGATIWNTFPGIPGVETMVPILVSEGLNKGRISLSRFVEVTSRNAAIHYGIYPKKGSMEIGSDADFTVIDLEKEYVIDEQKTESMAKYNPLHGMKLKGKPVQTIIRGSVVFEEDKGIVGEAGYGEYVPRQSIQRLERTLKYEKYEAVNVKTKDPVAQQ

SEQ ID NO: 179 - protein, 471 aa, Anaerovirgula multivorans
  4-ureido-5-carboxyl-imidazole-amide hydrolase
MKYDVIVKNARIPQGDDTVLTHILVKDEKIAGFLTDLEGIEYDKEINAEGHLTIPGCFDSHTHFMDPGFTHRENFLTGTSSAAAGGLTTIMDMPCCSKPSVRGVEELESKLNAIKDKAIIDYAMWGGVTGEDIRAGKKEIIKEQADYGVCAYKVYMTPSVPTYERVTDPEMLEAFRWVAETGLPVGIHAENYAMCDYYVNKFQKEGRLDGPAWAEARLELAEKAAIQLGISFAEETGARLHIVHMSTGIGAKLVGEAKKKGLDVTSETCPHYLTLNYQDAMTEHRQYAKIAPPLRTKRDNEELWEGLQNGSVDFMGTDHAPYEIATEKEAEGVNIWTAFPGIPGVETLVPIIVSEGYNKGRLTLSKMVEVLSTNAAKQYGLYPKKGAMHIGSDADFTIIDLEKEWTIDQKKTYSMAKYNPLHGIKLKGKPVKTVVRGTLVYDDEIGIVGEEGFGQFVRRQSIQKLDRVIKY

SEQ ID NO: 180 - protein, 471 aa, Carbydothermus hydrogenoformans
  4-ureido-5-carboxyl-imidazole-amide hydrolase
MGFDLVLKNAKIPQGNRTVDTHILVKDGKIAGFTNDLTGIEVAEVIDAENNLVLPGCIDSHTHFNDPGFTHRENFATGTQAAAAGGVTTIIDMPCCSIPSVRSVDNLEAKLEVIKDKAYVDYAMWGGATGEDVRNNALYNIKEQADYGVVAFKVYMTPSVPTYPPASDAEMLEIFREVAKTGLPVGIHAENLDLCTYHVNKLRNEGRFDGPAWAEARLELAEKVAIQMAISFAEETGARLHIVHMSTGIGGVLVGEAKKRGLDVTAETCPHYLVLNAYESMTKWRQFAKIAPPLRTTRDNEMLWEQLADGRVDFIATDHAPYEIATEKEAEGMDIWTAFPGIPGVETMVPIIISEGYNKGRLSLSRLVDVLSRNAAIHYGLYPKKGALEIGSDADFTVIDLDYEWTIDKDKMYTMAKYTPFHGMKLKGKPVKTIVRGKLVYDDKNGIVGKPGYGQFVKRQTISKLERWIKF

SEQ ID NO: 181 - protein, 470 aa, Sporanaerobacter acetigenes
  4-ureido-5-carboxyl-imidazole-amide hydrolase
MYDVIVKNARIPQGHDTVLTNILVKDGKIAGFVESIEGLEGREIIDAKGNLTIPGCIDSHTHFMDPGFTHRENFLTGTSGAAAGGVTTIIDMPCCSVPSVRSVAQLENKIEKIKDKAIVDFALWGGVTGEDVREGWLHNVQEQADYGVTGFKVYMTPSVPTYPRVTDPEMYECFKAVAKTGLPVGVHAENFAMCDFYVNRFQEEGRMDGPAWAEARMILAEKVAIQLAISFAEDTGVRLHIVHMSTGIGADLVKEAKMRGLDVTAETCPHYLTLNYEETMSKYVQLAKIAPPIRTTEDNERLWKGIQEGSVDFIATDHAPYSLETEKLKEGFNIWTAFPGIPGVETMAPIIISEGYNKGRISLSRLVEILSTNAAKHYGLYPKKGIMNIGSDADFTIVDIDKEWTIEKDKMYCLNKYTPFDGFKLKGKIYKTIVRGKVVYEDGEGIVGEEGYGQFVKRQSIQKLDHIIKF

SEQ ID NO: 182 - protein, 532 aa, Proteiniborus sp.
  4-ureido-5-carboxyl-imidazole-amide hydrolase
MYDLILKNARIPQGDYTVLTNILVKDEKIAGFTDCIDGIEAKNTIDAHGHLTLPGCIDSHTHFMYQGFPHRENFLTGTAAAAAGGITTVIDMPCCSVPSVRSVDQLNLKKDLCAPQALVDFALWGGVTGEDVRENRLHNVQEQADAGVVAFKVYMTPSVPTYPRVTDPEMYECFKAVAKTGLPIGIHAENFAMCDFYVKKFREEGRMDGPAWAEARMELAEKVAIELGISFAEASGARLHIVHMSTGIGAKLVGEAKKRGLNVTSESCPHYLTLNYQESMTIHGANAKIAPPLRTKRDNEEQWEGIRNGSIDFIATDHAPYELESEKIKEGTTIWTAFPGIPGVETMVPVIVSEGYNKGRISLSKLVDILSTNAAKHYGLYPKKGALNIGSDADFTIIDLDKTWTIDPKKQASMCGYTPLEGMELKGKVVKTIVRGHLVYVDEDASVLSEMTDEDLKKLVHDYPEGVAEKYKEHFDLYPQLYSAEYENSYRKNHPEVIDQHIRSIRGIKVKPGFGKFVKRQSIQVLPRTITY

SEQ ID NO: 183 - protein, 486 aa, Bacillus sp.
  4-ureido-5-carboxyl-imidazole-amide hydrolase
MDLILKNANIPQGDRQVLTNILVNEGKIVGYTNDISFLNASKVIDIQGKLVVPGCIDPHTHFMDPGFTHRETFATGSRSAAAGGLTTIIDMPCCSKPSVRDGDSFNKKIGPIRDQAYIDYCFWGGMTGEDAREGWIDNIYPQIDQGVVAFKVYMTPSVPTFPKVSDAEMLEIFKNVAKTGLPIGVHAENYDICTFNSKKLEKEGRLDGPAWAEARSSLAEKVAIELILSFAEATDARVHVVHMSTKEGVELVKAAKKRGVKVTAETCPHYLVLNAKDSMSERGSYAKIAPPLRGKEDNESHWQALADGTIDFVGTDHAPYEIATEKDYPGATIWNTFPGIPGVETMVPILVSEGLNKGRLSLSRFVEVTSRNAAIHYGIYPKKGSMEIGSDADFTVIDLEKEYVIDEQKTESMAKYNPLHGMKLKGKPVQTIIRGNVVFEEDKGVVGEAGYGEYVPRQSIQRLERTFKYEKYEAVNVKTKDPVAQQ

SEQ ID NO: 184 - protein, 465 aa, [Clostridium] ultunense Esp
  4-ureido-5-carboxyl-imidazole-amide hydrolase
MYQVIVKNARIPQGDETVLTNILIKDERIAGFVDGIDGIEAEEIIDAEGHLTLPGCIDSHTHFMDPGFTHRENFLTGTSSAAAGGLTTIVDMPCCSVPSVRSIPELENKLEKIKDKAIIDYGFWGGVTGEDVREGWLHNVKEQADYGVIGFKVYMTPSVPTYPRVTDPEMYEAFRTVAETGLPIGIHAENFAMCDYFVKKFQKEGRMEGPAWAEARMELAEKVAIQLGISFAEDTGARLHIVHMSTGIGAKLVKEAKLKGLDVTSETCPHYLTLNYEEAMSEYGAFAKIAPPLRTKRDNKELWEGLRDGSVDFIATDHAPYHIEDEKAKKGMNIWTAFPGIPGVETMVPIIVSEGYNKGRISLSKMVEILSTNAAIHYGLYPKKGSLNIGADADLTIIDLDKEWAIDQEKTYSMAKYNPLHGLKLKGKVAKTVVRGKVVFDEKEGITVKEGYGQFVKRTEYTKIR

SEQ ID NO: 185 - protein, 493 aa, Virgibacillus indicus
  4-ureido-5-carboxyl-imidazole-amide hydrolase
MDLILKNAVIPQGDRKVETNILIDNGEIVGFTNAVNGLAADEIIDIKGNLVVPGCIDPHTHFMDPGFTHRETFTTGTRSAAAGGITTHIDMPCCSKPSVRDKESFHKKIEPLRDQAYIDYCFWGGMTGEDVREGLLDNIYPQIEEGVVAFKVYMTPSVPTFPKVTDAEMLEIFSKIAPTGMPIGVHAENYEICTFYTEKMKKEGRMDGPAWAEARMPLAEKVAIQLIISFAEATGARVHVVHMTTKEGVELVREAKKRGVKVTAETCPHYLVLNAQDSMTERGTFAKIAPPLREKEDNIVHWQGLADGTIDFVGTDHAPYEIETEKDAPDSDIWNSFPGIPGVETMVPILVSEGLNKGRLSLSRFVEVTSRNAAIHYGIYPKKGSMELGSDADFTVIDLDREYTIDEQKTESMAKYNPLHGMELKGKPIQTIVRGKVVYDEDNGGIVGKSGYGEYVKRQSIQRLDRTLKYEVYEENAKEAESQPRREKALINN

SEQ ID NO: 186 - protein, 471 aa, Paenibacillus sp.
  4-ureido-5-carboxyl-imidazole-amide hydrolase
MSYDLIIKNANIPQGDRQVLTDILVKNGVIAGFAKGIKADAAEVIDAKGMLVLPGVIDSHTHFNDPGFTHREDFVTGTSSAAAGGVTMIVDMPCCSVPSVRSADNLFNKLNALEGKGIVDYAMWGGITGEDVRNNVLPEIISEQVANGVVAFKVYMTPSVPSYPRVTDPEMLEAFAAVSPTGLPIGIHAENFAICDYFTQKLKGEGRMDYPAWAEARMALAEKAAIQLGISFAEETGARLHIVHMSTAAGNELIREAKLKGTKVTAESCPHYLTLNAVDAMTEFGTFAKIAPPLRTAKDNEALWQGLVDGTIDFMATDHAPYEIATEKNAPGMDVWTSFPGIPGVETLVPIMISEGYNKGRLSLSRLVELLCTNPAVHYGLYPKKGALEIGTDADFTIVDLEKEWTLDKDQMHCKPKYTPFHGMKLKGKVDKTIVRGTLVFDEALGVIGKPGHGEYVKRQTISELPRLLKY

SEQ ID NO: 187 - protein, 552 aa, Andreesenia angusta
  4-ureido-5-carboxyl-imidazole-amide hydrolase
MYDLIIKNAKIPQGDETVMTNILVLEEKIAGFVDSTHGAQAKEVIDAEGHLVFPGFMDSHTHIMYHGFGHRENWVTGSAAAVRGGISTIIDMPCCTVPSTRTVKQLELKIDIANAQTVVDYGLWGGVTGEDVREGWMHNVQEQADFGVVAFKVYMTPSVPTYPRVTDPEMLECFKEVAKTGLPIGIHAENFAMCDFYVKKFQADGRMDGPAWSEARNELAEKVAIELGISFAEATGARLHIVHMSTGIGAKLIGEAKKRGINATSESCPHYLTLNYQDAMTKHGACAKIAPPIRTSGDNEEHWEGLRNGSIDFMATDHAPYELESEKTSEGTTIWNAFPGIPGTETMAPIIISEGLNKGRLSLSRTVEVLATNAAKHYGLYPKKGAMMVGSDADFAIVDLSKEWTIDPKTQASMCGYTPLEGMKLKGRIIKTIIRGTLVFDDAQEGVLPTLTDAELDKIVHNFPEGCAEKFADIFEQFPELASAEYDRSYRKLHPEKINEDIRRIKGIMVKPGFGKFVKRQSIQVLPKTITYNTTTPTGVNRREREDLPEFR

SEQ ID NO: 188 - protein, 471 aa, Paenibacillus sp.
  4-ureido-5-carboxyl-imidazole-amide hydrolase
MSYDLIIKNASIPQGDRQVLTDILVNNGVIVGFAKDIEADAAEVIDAGGKLVLPGVIDSHTHFNDPGFTHREDFVTGTASAAAGGVTMIVDMPCCSVPSVRSADNLFNKLNALEGKGIVDYAMWGGITGEDVRNNVLPEIISEQVANGVVAFKVYMTPSVPSYPRVTDPEMLEAFAAVSPTGLPIGIHAENFAVCDYFTNKLKGEGRLDYPAWAEARMALAEKAAIQLGISFAEETGARLHIVHMSTGAGNELIREAKLKGTKVTAESCPHYLTLNAVDAMTEFGTFAKIAPPLRTAKDNEVLWQGLADGTIDFMATDHAPYEIATEKDAPGMDVWTSFPGIPGVETLVPIMISEGYNKGRLSLSRLVELLCTNPAVHYGLYPKKGALEIGTDADFTIVDLEKEWTLDKDQMHCKPKYTPFHGMKLKGKVDKTIVRGTLVYDEALGVVGKPGHGEYVKRQTISELPRLLKY

SEQ ID NO: 189 - protein, 473 aa, Paludifilum halophilum
  4-ureido-5-carboxyl-imidazole-amide hydrolase
MNLLLKNANIPQGDRQVKADILVRDGKITGVTSDADGFAADRVIDVDGKLVVPGCIDPHTHFMDPGFTHRETFTTGSRSAAAGGLTCHVDMPCCSKPSVRSKGELHQKLNPLKDQAYIDFAFWGGMTGEDVREGKLDNVWGQVEEGVTAFKVYMTPSVPTFPKVTDAEMLEIFYKIAETGLPVGIHAENFDICSFYTEKLKNMGRTDAPAWAEARMALAEKTAIQLIIAFSEETGARVHIVHMSTKEGAELVRDAKRRGVKITAEACPHYLTLNAEEHMTRFGTLAKIAPPLRGTEDNEVHWQALADGTIDFVGTDHAPYEIATEKVGEGLDIWNSFPGIPGVETMVPVMVSEGLNKGRLSLSRFVEVTSRNAAIHFGLYPKKGSMELGSDADFTVIDLEREYVVDQEKVQSMAKYTPFHGMKLKGKPVMTIVRGEVVYDEDNGGIVGCSGYGQFVTRQSNAQLNRVEGELVY

SEQ ID NO: 190 - protein, 531 aa, Proteiniborus ethanoligenes
  4-ureido-5-carboxyl-imidazole-amide hydrolase
MYDMILKNARIPQGDNTVLTNILVKDEKIAGFTDCVDGIEAKNVIDAGGHLTLPGCIDSHTHFMYQGFPHRENFLTGTAAAAGGITTIIDMPCCSVPSARSVDELKLKIDVCQPQALVDFAMWGGVTGEDVREGWLHNVQEQADYGVVAFKVYMTPSVPTYPRVTDPEMYEAFRAVAKTGLPIGIHAENFAMCDYYVKEFQKEGRMDGPAWAEARMELAEKVAIELGISFAEASGARLHIVHMSTGIGAKLVGEAKKRGLNVTSESCPHYLTLNYQESMTIHGANAKIAPPLRTKRDNEEQWEGIRNGSIDFIATDHAPYELESEKIKEGLNIWTAFPGIPGVETMVPVIVSEGYNKGRISLSKLVDILSTNAAKHYGLYPKKGALHIGSDADFTIIDLDKRWVIEPKKQASMCGYTPLEGMELQGRVEKTIVRGHLVYKDVDESTLASLTDEELKTIVHEYPEGVRERYPEIFKNFPNLYSAEYERSYRKEHPEVIDKHVRGIRGIQVKPGFGRFVKRQSIQVLPRTITY

SEQ ID NO: 191 - protein, 471 aa, Alkaliphilus metalliredigens
  4-ureido-5-carboxyl-imidazole-amide hydrolase
MKYDVIVKNARIPQGDDTIVTNILVKDEKIAGFLTDIEGVEFDKTIDAEGHLTIPGCFDSHTHFMDPGFTHRENFLTGSSSAAAGGLTMIMDMPCCSKPSVRSIPELESKINNIKEKAIIDFALWGGVTGEDVREGKKHIVQDQADYGVCAFKAYMTPSVPTYERVTDPEMLEAFRWVAETGLPVGIHAENYAMCDFYVKKFQAEGRMDGPAWAEARLVEAEQAAIELGINFAESAGARLHIVHMSSGIGAKLVGAAKKRGLDVTSETCPHYLMLNYQDAMTEHGAFAKIAPPLRTTKDNKELWEGLQNGSVDFMGTDHAPYEIETEKDAEGMNIWTAFPGIPGVETLVPIMVSEGYNKGRLSLSKLVDVLSTNAAKQYGLYPKKGAMHIGSDADFTIIDLEREWTIDQKKTYSMAKYNPLHGIKLKGKPVKTVVRGTVVYDDAIGIVGKEGFGQFVKRQNIQKLDRVIKY

SEQ ID NO: 192 - protein, 490 aa, Fictibacillus solisalsi
  4-ureido-5-carboxyl-imidazole-amide hydrolase
MDLILKNAMIPQGDRTVITNILVHEGRITGFCNDIQTFNADKVLDMEGKLVVPGCIDPHTHFMDPGFTHRETFATGSRSAAAGGLTTIIDMPCCSKPSVRDGESFQKKIGPIRGQAYIDYCFWGGMTGEDARNGWIDNIYPQIDEGVVAFKVYMTPSVPTFPKVSDGEMLEIFQKVAKTGMPIGVHAENYDICTFYTEKLKNEGREDGPAWAEARMALAEKVAIELIISYAEATDARVHVVHMSTKEGVKLVREAKKRGVKVTSETCPHYLMLNAQDSMTERGTFAKIAPPLRGKDDNQAHWQALADGTIDFVGTDHAPYEIATEKAAADSSIWNSFPGIPGVETMVPILVSEGLNKGRLSLSRFVEVTSKNAAVHYGIYPKKGAMEIGSDADFTVIDLDHEWIIDEKQTESMAKYNPLHGIKLKGRPVQTIVRGTVVYDYEKGGITGTAGYGEYVKRQSIQRLERTLKYENYEIANLPSAARQQLEKSK

SEQ ID NO: 193 - protein, 486 aa, Sporosarcina globispora
  4-ureido-5-carboxyl-imidazole-amide hydrolase
MDLILKNANIPQGDRQVLTNILVNEGKIVGCTNDISFLNAAKVIDIQGKLVVPGCIDPHTHFMDPGFTHRETFATGSRSAAAGGLTTIIDMPCCSKPSVRDGESFNKKIGPIRDQAYIDYCFWGGMTGEDAREGWINNIYPQIDQGVVAFKVYMTPSVPTFPKVSDAEMLEIFKNVAKTGLPIGVHAENYDICTFNSKKLEKEGRLDGPAWAEARSSLAEKVAIELILSFAEATDARVHVVHMSTKEGVELVKAAKKRGVKVTAETCPHYLVLNAQDSMSERGSYAKIAPPLRGKEDNESHWQALADGTIDFVGTDHAPYEIATEKDYPGATIWNTFPGIPGVETMVPILVSEGLNKGRLSLSRFVEVTSRNAAVHYGIYPKKGSMEIGSDADFTVIDLEKEYVIDEQKTESMAKYNPLHGMKLKGKPVQTIIRGNVIYEEDKGIVGEAGYGEYVPRQSIQRLERTLKYEKYEAVNVKAKDPVAQQ

SEQ ID NO: 194 - protein, 486 aa, Bacillus firmus; identical to SEQ ID NO: 159
  4-ureido-5-carboxyl-imidazole-amide hydrolase

SEQ ID NO: 195 - protein, 486 aa, Paenibacillus sp.
  4-ureido-5-carboxyl-imidazole-amide hydrolase
MDLILKNANIPQGDRQVLTNILVNEGKIVGYTNDISFLNASKVIDIQGKLVVPGCIDPHTHFMDPGFTHRETFATGSRSAAAGGLTTIIDMPCCSKPSVRDGDSFNKKIGPIRDQAYIDYCFWGGMTGEDAREGWIDNIYPQIDQGVVAFKVYMTPSVPTFPKVSDAEMLEIFKNVAKTGLPIGVHAENYDICTFNSKKLEKEGRLDGPAWAEARSSLAEKVAIELILSFAEATDARVHVVHMSTKEGVELVKAAKKRGVKVTAETCPHYLVLNAKDSMSERGSYAKIAPPLRGKEDNESHWQALADGTIDFVGTDHAPYEIATEKDYPGATIWNTFPGIPGVETMVPILVSEGLNKGRLSLSRFVEVTSRNAAIHYGIYPKKGSMEIGSDADFTVIDLEKEYVIDEQKTESMAKYNPLHGMKLKGKPVQTIIRGNVVFEEDKGIVGEAGYGEYVPRQSIQRLERTFKFEKYEAVNVKTKDPVAQQ

SEQ ID NO: 196 - protein, 470 aa, Alkaliphilus sp.
  4-ureido-5-carboxyl-imidazole-amide hydrolase
MYDVLITNARIPQGDDTKLTNILVRDGIIEGFVQDIEGLQAKEIIDAEGHLTLPGCIDPHTHINDPGFTHRENFFTGTSAAATGGITMIIDMPCCSKPSVRSVEQLESKVNAIKNKAIVDYGMWGGVTGEDVRNDWLFNVREQADFGVLAFKVYMTPSVPTYPRVSDPEMLEAFKAVSETGLVVGVHSEIYAICEYYTKKFEREGRTDAPAWAEARKAISEKLAIQMGIYYAEEVGARFQVVHMALGEGARMVREAKMRGVDVVAETCPHYLLFNYQESMTKHGVFAKIAPPLRTKKDNELLWKGLQDGSIDFIGTDHAPYEIATEKTAAGLHIWNAFPGIPGVETMVPVMVSEGYNKGRLSLSRLVELLCKNAAINFGIYPKKGSMFVGTDADFTIIDLDREWTIKNEEMVTMAKYGPFDGMKLKGKPVKTIVRGKLVYDDEKGIVGKQGYGKLIKRQNENKLQKILKF

SEQ ID NO: 197 - protein, 471 aa, Alkaliphilus oremlandii
  4-ureido-5-carboxyl-imidazole-amide hydrolase
MKYDVIIKNARIPQGDDTMLTNILVKDEKIAGFLQDLEGIEYGREIDAEGNLTIPGCFDSHTHFMDPGFTHRENFLTGTSSAAAGGITTIMDMPCCSKPSVRSIPELESKLNAIKDKAIIDFAFWGGVTGEDVRNDNLEHVKAQADYGVCAFKVYMTPSVPTYERVTDPEMLEAFRAVAKTGLPVGIHAENYAMCDYYVKKFQKEGRLDGPAWAEARLELAEKAAIQLGISFAEETGARLHIVHMSTGIGAKLVGEAKKKGLDVTSETCPHYLTLNYQDAMTEHKQYAKIAPPLRTKRDNEELWEGLQNGSVDFIATDHAPYEIETEKEAEGVNIWTAFPGIPGVETMVPILVSEGYNKGRLTLSRLVEVLSTSAAKQYGLYPKKGAMHIGSDADFTIIDLNKEWTIDQKKTYSMAKYNPLHGIQLKGKPVKTVVRGTLVYDDKEGIVGKEGFGQFVKRQNIQKLDRVIKY

SEQ ID NO: 198 - protein, 471 aa, Caloramator mitchellensis
  4-ureido-5-carboxyl-imidazole-amide hydrolase
MRYDLIVKNALIPHGDETVLTNILVKDEKIAGFVDKIEGIEADNVIDAEGHLTLPGCIDSHTHYMDPGFTHRENFLTGTSGAAVGGVTTIIDMPCCSVPSVRGVAELHNKVNAIKDKAIVDYAMWGGVTGEDVRNKNLKIVKEQADEGVVAFKVYMTPSVPSYPRVTDPEMLEIFYEVSKTGLPVGVHAENFAICDYMVQKLREEGRMDGPAWAEARMELAEKVAIQLCISYAEETGARFHVVHMSTGIGAKLVKEAKMRGIDVTSETCPHYLTLNYQEHMTKFKQFVKIAPPIRTKKDNEELWQGLIDGSVDFIATDHAPYEIPTEKDAPGMTIWTAFPGIPGTETMVPIIVSEGYNKGRLSLSKLVEILSTNAAKHYGIYPKKGAMMIGSDADFTIIDLNKEWTIEKEKMQCLNKYTPFDGMKLKGKVAKTIVRGTLVYDDEKGIVGKPGYGQWIKRQNIQKLERKIKF

SEQ ID NO: 199 - protein, 480 aa, Clostridium cylindrosporum
  4-ureido-5-carboxyl-imidazole-amide hydrolase
MKFDVIVKNARIPQGHANETVVTNILIKDEQIAGFVNCIDGIDAVNVIDAAGNLVLPGCIDSHTHFMDPGFSHRETVESGSQQAARGGITTVIDMPCCTVPSARNVDQFNWKKEYWSEHCHVDFALWGGVTGEDVRLKQLHVVKDQADAGAVAFKVYMTPSVPTYPRVTDPELLEIAKAVAETGLSMGVHAENFAMCDFYVNKLKEEGRLDGPAWADARLELAEKVAIELGISISEATGVRYHVVHMSTSIGAKLIRQGKMRGLDVTSETCPHYLTINAQDAMTEYHACAKIAPPLRTKKDNAELWEGLANGSVDFIATDHAPYNIELEKTGRDIWTAFPGIPGIETMVEIIVNGFNDGKITLEKMVEILSKNAAIHYGLYPKKGSMQVGSDADLTIIDLDKEWTIDKDDKDSYMQNKYTPLQGIKLKGRVNKTIVRGHLVYEEGTGIISEAGFGNFIPRQNEDKLERKLRFIEYKPTTF

SEQ ID NO: 200 - protein, 469 aa, Ammoniphilus sp.
  4-ureido-5-carboxyl-imidazole-amide hydrolase
MDLILKNANIPQGDRQVRTNILVKDEKIVGFIDSVDGIQAKQVIDMEGKLVVPGCIDPHTHFMDPGFEHRENFATGTMSAAAGGVTTIIDMPCCSKPSVRSEDSFYAKLNPIKEKAYIDFAFWGGMTGEDVREGWLDNVWGQIEAGVTAFKVYMTPSVPTFPKVTDAEMLEIFYKVAETGLPIGIHAENYDICSFYTEKLKNEGRTDGPAWAEARKALAEKVAIELIISYVEETGARAHIVHMSTKEGAELIRRAKERGVKITAETCPHYLMLNAQDSMTEHGTFAKIAPPLRGKEDNEVHWKALADGTIDFVGTDHAPYEIATEKAAEDSTIWNSFPGIPGVETMVPILVSEGLNKGRLSLSRFVEVTSRNAAIHFGLYPKKGAMEIGSDADFTVIDLDKEGVIDQEKTYSMAKYNPLHGIKLKGMPVMTIIRGNVVFEDGKGIVAEEGFGQYTKRQSPTQLQSTLKF

SEQ ID NO: 201 - protein, 471 aa, Carbydothermus pertinax
  4-ureido-5-carboxyl-imidazole-amide hydrolase
MGFDLVLKNAKIPQGDRTVDTHVLVKDGKIAGFTTDLTGIEVAEVIDAENNLVLPGCIDSHTHFNDPGFTHRENFSTGTQAAAAGGVTTIIDMPCCSVPSVRSVDNLEAKLEVIKDKAYVDYAMWGGATGEDVRNNALYNIKEQADYGVVAFKVYMTPSVPTYPPASDAEMLEIFREVAKTGLPVGIHAENLDLCTYHVNKLRKEGRMDGPAWAEARLELAEKVAIQMAISFAEETGVRLHIVHMSTGIGGVLVGEAKKRGIDVTAETCPHYLVLNAYESMTKWRQFAKIAPPLRTTRDNEMLWEQLADGRVDFIATDHAPYEIPTEKEAEGMDIWTAFPGIPGVETMVPVIISEGYNKGRLTLSRLVDVLSRNAAIHYGLYPKKGALEIGSDADFTVIDLDYEWTIDKDKMYTMAKYTPLHGMKLKGKPVKTIVRGKLVYDDKNGIVGKPGYGQFVKRQTISKLERWIKF

SEQ ID NO: 202 - protein, 533 aa, Soehngenia sp.
  4-ureido-5-carboxyl-imidazole-amide hydrolase
MMYDVIIKNARIPQADETTLTNILVKDEKIAGFIDNIDGIDAKEIIDANGHLTLPGCIDSHTHFMYQGFPHRENFLTGTASAAAGGVTTIIDMPCCSVPSVRSVEQLKLKLDAVSPQALVDFAFWGGVTGEDVRNGWLHNVEEQAEYGVVAYKAYMTPSVPTYPRVDDPEMLEAFTEVAKTGLPIGIHAENYAMCDYYVKKLQREGRTDGPAWAEARLELAEKVAIELGISFAEHTGARLHIVHMSTGIGAKLIGEGKKRGLNVTSETCPHYLTLNYQEAMTKYGPLAKIAPPLRTKKDNEELWEGLRNGSVDFMATDHAPYELESEKFSKGMNIWTSFPGIPGVETMVPVLISEGYNKGRLSLSKLVEVLSTNAAKHYGIYPKKGAMNIGSDADFTIIDLDKRWKVDPKKLASMCGYTPLEGMELKGKVAKTILRGTLIYEDNDESELAKMTDEDLQRIVHNYPEGLEERYKDILKEYPHLHSKEYENSYRKNHPEVIDKHIRGIRGIKVKPGFGQFVKRQSIQVLPRRLTF

SEQ ID NO: 203 - protein, 59 aa, Soehngenia saccharolytica
  4-ureido-5-carboxyl-imidazole-amide hydrolase
MAIDQSRIRNFCIIAHIDHGKSTLADRIIESTGTLTSREMQAQVLDNMDLERERGITIK

SEQ ID NO: 204 - protein, 491 aa, Natribacillus halophilus
MDLILKNAHIPQGDRQVLTNVLVDQGTIVGFTNSIEGLEADEVFDVQQKLVVPGCIDPHTHFMEPGFNHRDTFDAAGRSAAKGGLTSHIDMPCTTKPSVRSREEFYEKLNSIKEKAVIDFAFWGGMTGEDVREEKLHNIQPQIDEGVVAFKVYMTPSVPTFPPVSDAEMLEIFRHVAPTGLPIGVHAENLDICTFYSNKLEKEGREDPVAWSEARGVIAEKTAIELILSFAEETDARVHVVHMTSKKGVELVREAKKRGVKVTSETCPQYLVLNAEEAMNKWGTFAKIAPPLRGKEDNEAFWQGLADGTVDFVGTDHAPYNIETEKKGSTIWTSFPGMPGVEQMVPILVSEGLNKGRLSLSRFVEVTSRNAAIHYGLYPKKGAMELGSDADFTVIDLEREYTIDKDNVLTDAKYTPFHGMELKGIPVQTIVRGKTVYNADDGGIVSEPGYGKYVERQSIQHLTRELKYETENTLGKPIKVQEKEKQSAGGI

SEQ ID NO: 205 - protein, 61 aa, Unknown
  compost metagenome 4-ureido-5-carboxyl- imidazole-amide hydrolase
MTIVRGNVVYEDGKGIVGQEGYGQYVKRQSTQQLDAVINLTKSNESKAVSQSSEKKESVKL

SEQ ID NO: 206 - protein, 60 aa, Unknown
  compost metagenome xanthine amide hydrolase
MHCKPKYTPFHGMKLKGKVDKTIVRGTLVYDEALGVVGKPGHGEYVKRQTISELPRLLKY

SEQ ID NO: 207 - DNA, 1828 bp, Artificial Sequence
  promoter
ggcccattcggggcgaaatcgatctcggaaatcggtgtaaatggcgccgctccggcgattgctaccgcaattcacgatgcctgcggcatctggttacgcgaatggcatttcacaccggagaaaatactcaccgcgctggaaaaaatataagtaataaatggccgcttcggcggcctgtttctctgtttgaacgatgactaaatagtgactgcgaaaatgagataagccgttctcaaggcgtcctgcgccccaaattcgtgggaatttattttactaattcagatgatcaaatttactttaaaaggagtgaagggtgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatagtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaactccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcattgctcacatctcactttaatcgtgctcacattacgtgactgattctaacaaaacattaacaccaactggcaaaattttgtcctaaacttgatctcgacgaaatggctgcacctaaatcgtgatgaaaatcacatttttatcgtaattgccctttaaaattcggggcgccgaccccatgtggtctcaagcccaaaggaagagtgaggcgagtcagtcgcgtaatgcttaggcacaggattgatttgtcgcaatgattgacacgattccgcttgacgctgcgtaaggtttttgtaattttacaggcaaccttttattcactaacaaatagctggtggaatatatgtctgatataaaccatgcaggttctgaccttatatttgaactggaggatcgccctccctttcatcaggctctcgtaggtgccattacccatctgttggcaattttcgttccgatggtaacccccgcgttaatcgtgggtgcggccttacagctttccgctgaaacaactgcctatcttgtttctatggcgatgatcgcctctggtattggtacctggttacaagtaaaccgctacggcatcgtcggttctggtttactttcaattcagtcagtcaatttttcatttgttacggtcatgattgcgctgggtagcagc

SEQ ID NO: 208 - DNA, 5666 bp, Artificial Sequence
  the nucleic acid molecule encoding xanthine amide hydrolase and 4-ureido-5-carboxyimidazole amide hydrolase
gcgtatccctggcgacagtatccagggtcttaaatgacgatccaacattgaatgtgaaagaagagacgaaacatcgcattctggagatcgccgaaaagctggagtacaagaccagtagcgcccgtaaactccagacaggtgcagtcaaccaacatcatattctggctatctacagctaccagcaggagctggagatcaacgatccttactatctggcgatccgccacggcattgaaactcagtgcgaaaagctggcgatcgaactgaccaactgttatgaacacaacggtttaccagacattaaaaacgtcaccggtattttaattgtcggcaaacccacgcccgccctgcgcgccgccgcctgtgcgttgaccgacaatatctgttttatcgactttcacgaacccggcagcggttacgatgcggtggatatcgatctggcacgtatcagtaaagaaatcatcgatttctatatcaatcagggcgttaatcgtattggttttattggcggtgaagacttacccggcaaggcggatattcgtgaggtcgcctttgcggaatatggtcgtctgaaacacgtggtacgcgaagaggatatctggcgcggcggtttttccagttcgtcgggctatgaactggcaaaacagatgctggcgcaggaagactatccgaaggcactgtttgttgcttccgattccattgctatcggcgtactgcgggcaattcatgaacgtggcctgaacatcccacaggatatttcgcttatcagcgttaacgatatccccaccgcgcgatttacctttccgccgctctccaccgtgcgcatccattccgagatgatgggaagtcagggcgttaacctggtgtatgaaaaagcccgcgatggtcgcgcgctgccgctgttggtcttcgttcccagcaaattaaaactgcgcggcacgacccgttaaatccccttgcacgctgtccagcaatcgtttttgccggacagtgctgccgtcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatgttttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtttgctcacatctcactttaatcgtgctcacattacgtgactgattctaacaaaacattaacaccaactggcaaaattttgtcctaaacttgatctcgacgaaatggctgcacctaaatcgtgatgaaaatcacatttttatcgtaattgccctttaaaattcggggcgccgaccccatgtggtctcaagcccaaaggaagagtgaggcgagtcagtcgcgtaatgcttaggcacaggattgatttgtcgcaatgattgacacgattccgcttgacgctgcgtaaggtttttgtaattttacaggcaaccttttattcactaacaaatagctggtggaatatatggatttaattttgaaaaatgcaagcattccccagggagatcgtcaggttttaacaaatattctggtaaacgagggaaaaattgttggctacactaatgacatttcttttctaaatgcagccagggtaattgatattaaaggaaaattagtcgttccaggctgtattgatccgcatactcattttatggatccaggcttcactcatagagaaacctttgctactggcagccgttctgcagcagccggaggcttaacaaccattatagatatgccttgctgcagcaagccatccgttcgtgatggggagagtttcaataaaaaaatcggaccaatcagggatcaggcctatattgattactgtttctggggaggcatgactggagaagatgctcgtgaaggctggattgataatatttatccgcagattgaccagggagttgttgcctttaaagtttacatgactccttctgtacctaccttccctaaagtatcggacgcagagatgctggaaattttcagtaacgttgcaaaaaccggactcccaattggtgtacatgctgaaaactatgatatctgcacattctattccaaaaaacttgaaaaggaagggcgccttgacggaccagcatgggcggaagcgcgttcaagcctcgctgagaaggtggccattgaattgattctcagctttgcagaagctacagacgcacgtgtacatgtggtacatatgagcacaaaagaaggcgtcgagctggtaaaagcagccaagaagcgtggtgttaaggttacagcagaaacctgtcctcactatcttgtacttaatgcaaaggactccatgtcagagcgcggttcctatgcaaagattgctcctcctttacgaggaaaagaggataacgaatctcactggcaggctctggctgacggaaccatcgattttgtcggtacagatcatgctccatatgaaattgccacagaaaaggattatcctggggcaacgatctggaacactttccctggcatacccggggtagaaacgatggtgccgatccttgtcagtgaaggtttaaataaaggccgtttatccctatcaagatttgtcgaggtaacgagccgcaatgccgccattcactatggcatttaccctaaaaagggatcaatggaaattggcagtgatgctgatttcactgttattgatcttgagaaagaatatgtaatagacgagcaaaaaacagaatcaatggctaaatataaccctctgcatggcatgaagcttaaaggaaagccagtccaaacgatcattcgcggaaacgtgattttcgatgaagataaaggcatcgtaggagaagcgggctatggtgagtatgttccgcgccaaagcattcagaggcttgagagaacattcaagtatgaaaaatacgaagcggtaaaagcaaaaacaaaagacccagtagcacagcaataacttaaaaaataaggaggattacacatatggcccgttatggcatttgtgttattgatatgctgaatgatttcatcggcgaaagtgcaccgctgcgttgcccgggtggcgaagaaattgtgccgaatctgcagaaactgtttaaatgggcccgcgcccgtatggatgcaggtcatgatgatgtgcatctgattcatattcaggaagcccatcgcaaacatgatgcagattttcgtgtgcgtccggttcatgcaattaagggcacccacggtagtgattttattaaggaactgtatccggaagatcgccgttgcgaatatattattccgaaacgccgccatagcgcctttcagcataccgatctggatctgtatctgcgcgaagaaaatctggataccgttgttattaccggcgtttggaccaatgtgtgctgtcgcagtaccgccgcagatgccctggccaatgcatatcgcgtgattaccctgaccgatggtaatcatagcaaaacccaggcacagcatgaaagcggtctggttgatctgagcatttttaccaaactgaccaccattgatcagtttaccgccgatattgatgcaggtgttgatgcctgggcaggcgcaggtgaccagaaaaatgaagttaaataaggtacccaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggccttttttattttcgtgatccagttaaagtaaatgcatttactcgctactttttagtaaaatttttactaaactccccagcaattacacaaactaccatcaccatgaatggttccgatttctctctaccgggaggccctatgaatcgctgggaaaacattcagctcacccacgaaaaccgacttgcgccgcgtgcgtactttttttcatatgattctgttgcgcaggctcgtacttttgcccgtgaaaccagcagcctgtttctgcccttaagcggtcagtggaatttccacttttttgaccatccgctgcaagtgccagaagccttcacctctgagttaatggctgactgggggcatattaccgtccccgccatgtggcaaatggaaggtcacggcaaactgcaatataccgacgaaggttttccgttccccatcgatgtgccgtttgtccccagcgataacccaaccggtgcctatcaacgtattttcaccctcagcgacggctggcagggtaaacagacgctgattaaatttgacggcgtcgaaacctattttgaagtctacgttaacggtcagtatgtggggttcagcaagggcagtcgcctgaccgcagagtttgacatcagcgcgatggttaaaaccggcgacaacctgttgtgtgtgcgcgtgatgcaatgggcggactctacctacgtggaagaccaggatatgtggtggtcggcggggatcttccgcgatgtttatctgatcggaaaacaactcacgcacattaacgatttcaccgtgcgcaccgactttgacgaagcctattgcgatgccacactttcctgcgaagtggtgctggaaaatctcgccgcctcccctgtcgtcacgacgctggaatataccctgtttgatggcgaacacgtggtgcacagcagcgccattgatcatttggcaattgaaaaactgaccagcgcccgctttgcttttactgtcgaacagccgcagcaatggtcagcagaa

(19) United States
(12) Patent Application Publication
(10) Pub. No.: US 2023/0049044 A1
(43) Pub. Date: Feb. 16, 2023

(54) 4-UREIDO-5-CARBOXYL-IMIDAZOLE-AMIDE HYDROLASE AND USE THEREOF

(71) Applicant: TIANJIN UNIVERSITY, Tianjin (CN)

(72) Inventors: Yan ZHANG, Tianjin (CN); Peishan LI, Tianjin (CN); Yang TONG, Tianjin (CN); Yiling HU, Tianjin (CN); Dazhi LIU, Tianjin (CN)

(21) Appl. No.: 17/793,009

(22) PCT Filed: Jan. 13, 2021
(86) PCT No.: PCT/CN2021/071497

§ 371 Date: Jul. 14, 2022

(30) Foreign Application Priority Data
Jan. 14, 2020 (CN) 202010036500.6
Jan. 14, 2020 (CN) 202010036519.0

(51) Int. Cl. C12N 9/86; C12N 15/70; A61P 19/06; A23L 33/13; A23L 33/175; A23L 33/18

(52) U.S. Cl. CPC C12N 9/86; C12Y 305/02017; C12N 15/70; A61P 19/06; A23L 33/13; A23L 33/175; A23L 33/18; C12N 2800/101; A61K 38/00

(57) ABSTRACT

Provided are a 4-ureido-5-carboxyl-imidazole-amide hydrolase and use thereof, particularly use in the treatment of gout.

### CROSS-REFERENCES TO RELATED APPLICATIONS

This application claims the priority of Chinese Patent Application No. 202010036500.6 and No. 202010036519.0 filed on Jan. 14, 2020, the entire contents of which are incorporated herein by reference in their entirety.

### TECHNICAL FIELD

This application generally relates to the field of biomedicine technology; specifically, this application provides a new 4-ureido-5-carboxyimidazole degradation pathway, enzymes involved in the pathway, and their usages, especially in the treatment of gout.

### BACKGROUND TECHNIQUE

The absorption of purines mainly occurs in the intestine, which is an anaerobic environment. Gout is a type of arthritis caused by abnormal purine metabolism. It is characterized by recurring red, tender, burning, and swollen joints. The pain usually occurs quickly, reaching maximum intensity in less than 12 hours. The cause of gout is the increase in the level of uric acid in the body, which causes the deposition of urate in the joints and kidneys, and uric acid is a product of purine metabolism.

The existing drugs for gout include colchicine, allopurinol, febuxostat and other non-steroidal anti-inflammatory drugs and glucocorticoids. Colchicine inhibits the chemotaxis, adhesion and phagocytosis of neutrophils, so as to control local pain, swelling and inflammation in the joints. Allopurinol and febuxostat are selective xanthine oxidase inhibitors that can treat gout by reducing the blood urate concentration, but the above drugs have relatively large side effects.

The development and application of new drugs for the prevention, intervention and/or treatment of gout are needed in the art.

### SUMMARY OF THE INVENTION

In the first aspect, the present application provides polypeptide A, which comprises the amino acid sequence shown in SEQ ID NO:1 or a functional variant thereof, wherein the functional variant has 4-ureido-5-carboxyimidazole amide hydrolase activity.

In some embodiments of the first aspect, the polypeptide A has a catalytic site A defined as follows in spatial conformation:

The catalytic site A includes catalytic triads close to each other in spatial conformation.

In some embodiments of the first aspect, the catalytic triad is composed of C134, D10, and K101 with reference to SEQ ID NO:1.

In some embodiments of the first aspect, the catalytic site A further comprises a divalent metal ion.

In some embodiments of the first aspect, the catalytic site A further includes one divalent metal ion, such as Zn²⁺ or Mn²⁺.

In some embodiments of the first aspect, the catalytic site A further comprises four amino acid residues coordinated with the metal ion, such as H61, H74, E59 and D67.

In some embodiments of the first aspect, the polypeptide A further comprises a binding site A defined in space conformation: the binding site A includes F15, R71, V129, H104 and W130 amino acid residues of SEQ ID NO:1 that are close to each other in space conformation.

In some embodiments of the first aspect, wherein the distance between the catalytic site A and the binding site A is no more than 5 angstroms.

In some embodiments of the first aspect, wherein the functional variant is a natural isoenzyme of the amino acid sequence shown in SEQ ID NO:1.

In some embodiments of the first aspect, wherein the functional variant is the insertion, substitution and/or deletion of one or more amino acids on the basis of the amino acid sequence or its natural isoenzyme shown in SEQ ID NO:1.

In some embodiments of the first aspect, the insertion, substitution and/or deletion does not occur at the catalytic site A and/or the binding site A.

In the second aspect, this application provides a nucleic acid molecule that encodes the polypeptide A described in the first aspect, or encodes the polypeptide A and the polypeptide B described in the first aspect, wherein the polypeptide B comprises the amino acid sequence shown in SEQ ID NO: 103 or a functional variant thereof, the functional variant having xanthine amide hydrolase activity.

In the third aspect, the present application provides an expression cassette, which contains the nucleic acid molecule described in the second aspect.

In the fourth aspect, the present application provides an expression vector, which comprises the nucleic acid molecule described in the second aspect or the expression cassette described in the third aspect.

In the fifth aspect, the present application provides a host cell, which comprises the nucleic acid molecule described in the second aspect, the expression cassette described in the third aspect, or the expression vector described in the fourth aspect.

In some embodiments of the fifth aspect, the host cell can express and produce: polypeptide A, which comprises the amino acid sequence shown in SEQ ID NO:1 or a functional variant thereof, wherein the functional variant has 4-urea 5-carboxyimidazole amide hydrolase activity.

In some embodiments of the fifth aspect, the host cell can express and produce: polypeptide A, which comprises the amino acid sequence shown in SEQ ID NO:1 or a functional variant thereof, wherein the functional variant has 4-urea 5-carboxyimidazole amide hydrolase activity; and polypeptide B, which comprises the amino acid sequence shown in SEQ ID NO: 103 or a functional variant thereof, wherein the functional variant has xanthine amide hydrolase activity.

In some embodiments of the fifth aspect, the polypeptide A has a catalytic site A defined as follows in the spatial conformation:

The catalytic site A includes catalytic triads close to each other in spatial conformation.

In some embodiments of the fifth aspect, the catalytic triad is composed of C134, D10, and K101 with reference to SEQ ID NO:1.

In some embodiments of the fifth aspect, the catalytic site A further comprises a divalent metal ion.

In some embodiments of the fifth aspect, the catalytic site A further includes one divalent metal ion, such as Zn²⁺ or Mn²⁺.

In some embodiments of the fifth aspect, the catalytic site A further comprises four amino acid residues coordinated with the metal ion, such as H61, H74, E59 and D67.

In some embodiments of the fifth aspect, the host cell is a eukaryotic cell or a prokaryotic cell.

In some embodiments of the fifth aspect, the eukaryotic cell is a yeast cell.

In some embodiments of the fifth aspect, the prokaryotic cell is selected from the group consisting of Escherichia, Lactobacillus, Bifidobacterium, Bacteroides and Firmicutes

In the sixth aspect, this application provides a pharmaceutical composition or health food, which comprises the polypeptide A and optional polypeptide B described in the first aspect, the nucleic acid molecule described in the second aspect, the expression cassette described in the third aspect, and the expression vector described in the fourth aspect or the host cell and a pharmaceutically acceptable carrier or excipient described in the fifth aspect, wherein the polypeptide B comprises the amino acid sequence shown in SEQ ID NO: 103 or a functional variant thereof, The functional variant has xanthine amide hydrolase activity.

In some embodiments of the sixth aspect, the pharmaceutical composition or health food is used for the prevention, intervention and/or treatment of gout.

In the seventh aspect, this application provides the polypeptide A and optional polypeptide B described in the first aspect, the nucleic acid molecule described in the second aspect, the expression cassette described in the third aspect, the expression vector described in the fourth aspect, and the usage of the host cell according to the fifth aspect or the pharmaceutical composition or health food according to the sixth aspect in degrading 4-ureido-5-carboxyimidazole, wherein the polypeptide B comprises the amino acid sequence shown in SEQ ID NO: 103 or a functional variant thereof, the functional variant having xanthine amide hydrolase activity.

In some embodiments of the seventh aspect, the degradation of 4-ureido-5-carboxyimidazole occurs in vitro.

In the eighth aspect, this application provides the polypeptide A and optional polypeptide B described in the first aspect, the nucleic acid molecule described in the second aspect, the expression cassette described in the third aspect, the expression vector described in the fourth aspect, and the usage of the host cell of the fifth aspect or the pharmaceutical composition or health food of the sixth aspect in the preparation of a medicament for the prevention, intervention and/or treatment of gout, wherein the polypeptide B comprises the amino acid sequence shown in SEQ ID NO: 103 or a functional variant thereof, the functional variant having xanthine amide hydrolase activity.

In the ninth aspect, this application provides a method for preventing, intervening and/or treating gout, which comprises providing an individual in need the polypeptide A and optional polypeptide B of the first aspect, the nucleic acid molecule of the second aspect, the expression cassette of the third aspect, the expression vector of the fourth aspect, the host cell of the fifth aspect, or the pharmaceutical composition or health food of the sixth aspect, wherein the polypeptide B comprises the amino acid sequence shown in SEQ ID NO: 103 or a functional variant thereof, which has xanthine amide hydrolase activity.

In some embodiments of any of the above aspects, wherein the polypeptide B has a catalytic site B defined as follows in a spatial conformation:

The catalytic site B comprises amino acid residues close to each other in spatial conformation, referring to the amino acid residues of H59, H61, K151, H186, H242 and D316 of SEQ ID NO:103.

In some embodiments of any of the above aspects, the catalytic site B further comprises a divalent metal ion.

In some embodiments of any of the above aspects, the catalytic site B further includes two divalent metal ions (for example, Zn²⁺ and/or Mn²⁺).

In some embodiments of any of the above-mentioned aspects, wherein the polypeptide B further comprises a binding site B having the following definition in a spatial conformation:

The binding site B includes amino acid residues I288, A289, P338 and G339 of SEQ ID NO: 103 that are close to each other in spatial conformation.

In some embodiments of any of the above aspects, wherein the distance between the catalytic site B and the binding site B is not more than 5 angstroms

In some embodiments of any of the above aspects, wherein the functional variant having xanthine amide hydrolase activity is a natural isoenzyme of the amino acid sequence shown in SEQ ID NO: 103.

In some embodiments of any of the above aspects, wherein the functional variant with xanthine amide hydrolase activity has one or more amino acids insertion, substitution and/or deletion of the amino acid sequences shown in SEQ ID NO: 103 or its natural isoenzymes.

In some embodiments of any of the above aspects, the insertion, substitution and/or deletion does not occur at the catalytic site B and/or the binding site B.

### DESCRIPTION OF THE DRAWINGS

FIG. 1 shows the human body's metabolic pathways that degrade various purines and the EC numbers of enzymes that catalyze each step of the reaction.

FIG. 2 shows the metabolic reaction of xanthine oxidase degrading xanthine to uric acid and its catalytic mechanism involving complex Mo-containing prosthetic groups.

FIG. 3 shows the mechanism of allopurinol in the treatment of gout by inhibiting xanthine oxidase to prevent the production of uric acid.

FIG. 4 shows the reaction formula (1) of xanthine amide hydrolase degrading xanthine under anaerobic conditions and the reaction formula (2) 4-ureido-5-carboxyimidazole amide hydrolase degrading 4-ureido-5-carboxyimidazole under anaerobic conditions and the entire metabolic pathway.

FIG. 5 shows the electrophoresis results of the purified xanthine amide hydrolase, where lane M represents the molecular weight indicator, lane T is the bacterial lysate expressing xanthine amide hydrolase, and lane U represents the bacterial lysate after the Co affinity column, Lanes E1, E2, and E3 represent 1, 2 and 4 μg xanthine amide hydrolase purified and eluted by a cobalt affinity column, respectively.

FIG. 6 shows the LC-MS (liquid mass spectrometry) test results of the enzymatic activity of xanthine amide hydrolase catalyzing the hydrolysis and opening of xanthine, where A is the LC-MS test result of the negative control without enzyme, and B is the test group LC-MS detection results.

FIG. 7 is a diagram of the active center of the xanthine amide hydrolase structure obtained by computer simulation by the PHYRE2 server (PHYRE2 Protein Fold Recognition Server). The catalytic site contains four histidines, one carboxylated lysine, one aspartic acid and two divalent metal ions (such as Zn²⁺ and/or Mn²⁺) that are close to each other in spatial conformation; the binding site contains an isoleucine, an alanine, a proline and a glycine residue that close to each other in spatial conformation to interact with xanthine.

FIG. 8 is a phylogenetic tree diagram of all xanthine amide hydrolase enzymes in UniRef50_Q3AEA1. Gray markers are selected strains from each tree branch to represent Bacillus firmus, Clostridium cylindrosporum DSM 605, Clostridium purinilyticum, Carbydothermus hydrogenoformans strain ATCC BAA-161 DSM 6008 Z-2901 and Paenibacillus donghaensis, the amino acid sequences of xanthine amide hydrolase encoded by them are A0A366K523, A0A0J8G334, A0A0L0W692, Q3AEA1 and A0A2Z2KEH1, respectively, the sequences comparison is shown in FIG. 9.

FIG. 9 shows the sequence comparison results of xanthine amide hydrolase from five different species of Bacillus firmus, Clostridium cylindrosporum DSM 605, Clostridium purinilyticum, Carbydothermus hydrogenoformans, strain ATCC BAA-161/DSM 6008/Z-2901 and Paenibacillus donghaensis, the encoded accession numbers are A0A366K523, A0A0J8G334, A0A0L0W692, Q3AEA1 and A0A2Z2KEH1, wherein the catalytic site contains two divalent metals (such as Zn²⁺ or Mn²⁺, etc.) and four histidine (H), one lysine (K) and one aspartate (D) residues (solid box) coordinated with the gray background; the binding site contains an isoleucine (I), an alanine (A), a proline (P) and a glycine residue (G) that binds to xanthine with gray background.

FIG. 10 is the electrophoresis result of purified 4-ureido-5-carboxyimidazole amide hydrolase, where lane M represents the molecular weight indicator, and lane P is the bacterial lysate expressing 4-ureido-5-carboxyimidazole amide hydrolase. Lane T is the bacterial lysate supernatant expressing 4-ureido-5-carboxyimidazole amide hydrolase, lane U represents the bacterial lysate after TALON cobalt column affinity chromatography, and lanes E1, E2, and E3 represent 5, 15 and 20 g of 4-ureido-5-carboxyimidazole amide hydrolase of the bacterial lysate purified by cobalt affinity column.

FIG. 11 shows the reaction of 4-ureido-5-carboxyimidazole amide hydrolase catalyzing 4-ureido-5-carboxyimidazole, A is the control group, and B is the experimental group.

FIG. 12 is a diagram of the enzyme active center of the 4-ureido-5-carboxyimidazole amide hydrolase structure obtained by PHYRE2 server (PHYRE2 Protein Fold Recognition Server) computer simulation, wherein the catalytic site contains a cysteine (nucleophilic attack), an aspartate, a lysine which is close to each other in spatial conformation composed of a proteolytic enzyme-like catalytic triad, a bivalent metal ion (such as Zn²⁺ or Mn²⁺) and two histidines, one glutamate and one aspartate that form a coordination bond with the bivalent metal ion. The binding site contains an arginine, a phenylalanine, a tryptophan, a histidine, and a valine that interact with 4-ureido-5-carboxyimidazole in a spatial conformation close to each other.

FIG. 13 is a phylogenetic tree diagram of all 4-ureido-5-carboxyimidazole amide hydrolases of UniRef50_A6TWT6, UniRef50_A0A1H2QU42, UniRef50_Q3AEA0, UniRef50_A0A3R2KN38 and UniRef50_A0A0R3K3G8. Gray markers are selected from each tree branch to represent Alkaliphilus metalliredigens, Bacillus firmus, Paenibacillus donghaensis, Carbydothermus hydrogenoformans (strain ATCC BAA-161/DSM 6008/Z-2901), and Clostridium cylindrosporum. Their accession numbers are A6TWT6, A0A366K3D2, A0A2Z2KPK9, Q3AEA0, A0A0L0W6E3, respectively. Sequence comparison was performed in FIG. 14.

FIG. 14 shows the sequence comparison results of 4-ureido-5-carboxyimidazole amide hydrolase from six different species of Alkaliphilus metalliredigens, Bacillus firmus, Paenibacillus donghaensis, Carbydothermus hydrogenoformans (strain ATCC BAA-161/DSM6008/Z-2901), Gottschalkia purinilytica, and Clostridium cylindrosporum. Their accession numbers are A6TWT6, A0A366K3D2, A0A2Z2KPK9, Q3AEA0, A0A0L0W6E3 and A0A0J8D7U2, which contains one cystine (C), one aspartate (D), one lysine (K) with grey background that forming a proteolytic enzyme-like catalytic triad (solid box). A divalent metal ion (such as Zn²⁺ or Mn²⁺) and two histidines (H), one glutamate (E), and one aspartate (D) coordinated with the gray mark. The binding site contains an arginine (R), a phenylalanine (F), a tryptophan (W), a histidine (H) and a valine (V) (dashed box) on a gray background binding with 4-ureido-5-carboxyimidazole.

FIG. 15 shows the structure schematic diagram wherein the tandem integration of the nucleic acid molecule encoding 4-ureido-5-carboxyimidazole amide hydrolase and the nucleic acid molecule encoding xanthine amide hydrolase into the E. coli genome and the replacement of the xanthine transporter promoter for stable expression promoter.

FIG. 16 shows the detection results of the enzyme activity of xanthine amide hydrolase catalyzing the hydrolysis and opening of xanthine, where A is the extracted ion chromatogram (EIC) of the extracted products of the experimental group, control group 1 and control group 2, and B is the ESI chart of the mass spectrum of the product 4-ureido-5-carboxyimidazole in negative ion mode at a retention time of 2.993 min for the experimental group.

FIG. 17 shows the detection results of 4-ureido-5-carboxyimidazole catalyzed by 4-ureido-5-carboxyimidazole amide hydrolase. A is the extracted ion chromatograms (EIC) of the extracted products of the experimental group, control group 1 and control group 2, B is the ESI chart of the mass spectrum of the product 4-amino-5-carboxyimidazole in the negative ion mode at a retention time of 2.626 min for the experimental group.

### SEQUENCE DESCRIPTION

SEQ ID NO: 1 shows the amino acid sequence of the 4-ureido-5-carboxyimidazole amide hydrolase expressed by Clostridium cylindrosporum with the accession number A0A0J8D7U2.

SEQ ID NO: 2 shows the amino acid sequence of 4-ureido-5-carboxyimidazole amide hydrolase expressed by Sporanaerobacter acetigenes DSM 13106 with the accession number A0A1M5YT22.

SEQ ID NO: 3 shows the amino acid sequence of 4-ureido-5-carboxyimidazole amide hydrolase expressed by Thermosyntropha lipolytica DSM 11003 with the accession number A0A1M5LQD2.

SEQ ID NO: 4 shows the amino acid sequence of 4-ureido-5-carboxyimidazole amide hydrolase expressed by Aneurinibacillus migulanus with the accession number A0A0D1Y234.

SEQ ID NO: 5 shows the amino acid sequence of the 4-ureido-5-carboxyimidazole amide hydrolase expressed by the Alkaliphilus oremlandii strain OhILAs with the accession number A8MLK8.

SEQ ID NO: 6 shows the amino acid sequence of 4-ureido-5-carboxyimidazole amide hydrolase expressed by Fictibacillus enclensis with the accession number A0A0V8JCF9.

SEQ ID NO: 7 shows the amino acid sequence of 4-ureido-5-carboxyimidazole amide hydrolase expressed by Bacillus sp. FJAT-21945 with the accession number A0A0M0XDC1.

SEQ ID NO: 8 shows the amino acid sequence of 4-ureido-5-carboxyimidazole amide hydrolase expressed by Bacillus mesonae with the accession number A0A3T0HY92.

SEQ ID NO: 9 shows the amino acid sequence of 4-ureido-5-carboxyimidazole amide hydrolase expressed by Bacillus oceanisediminis with the accession number A0A562JUE6.

SEQ ID NO: 10 shows the amino acid sequence of 4-ureido-5-carboxyimidazole amide hydrolase expressed by Bacillus firmus with accession number A0A366K3D2.

SEQ ID NO: 11 shows the amino acid sequence of the 4-ureido-5-carboxyimidazole amide hydrolase expressed by Marinisporobacter balticus with the accession number A0A4V2SBF8.

SEQ ID NO: 12 shows the amino acid sequence of the 4-ureido-5-carboxyimidazole amide hydrolase expressed by Alkaliphilus peptidifermentans DSM 18978 with the accession number A0A1G5KWY0.

SEQ ID NO: 13 shows the amino acid sequence of 4-ureido-5-carboxyimidazole amide hydrolase expressed by Bacillus firmus with the accession number A0A0J5VZ19.

SEQ ID NO: 14 shows the amino acid sequence of 4-ureido-5-carboxyimidazole amide hydrolase expressed by Thermoflavimicrobium dichotomicum with the accession number A0A1I3TZC9.

SEQ ID NO: 15 shows the amino acid sequence of 4-ureido-5-carboxyimidazole amide hydrolase expressed by Bacillus oceanisediminis with the accession number A0A1S1YDU5.

SEQ ID NO: 16 shows the amino acid sequence of 4-ureido-5-carboxyimidazole amide hydrolase expressed by Paenibacillus sp. FSL R7-0331 with the accession number A0A089L6Q6.

SEQ ID NO: 17 shows the amino acid sequence of 4-ureido-5-carboxyimidazole amide hydrolase expressed by Caloranaerobacter sp. TR13 with the accession number A0A0P8X709.

SEQ ID NO: 18 shows the amino acid sequence of the 4-ureido-5-carboxyimidazole amide hydrolase expressed by Virgibacillus profundi with the accession number A0A2A2IDD7.

SEQ ID NO: 19 shows the amino acid sequence of 4-ureido-5-carboxyimidazole amide hydrolase expressed by Carboxydothermus islandicus with the accession number A0A1L8D5R7.

SEQ ID NO: 20 shows the amino acid sequence of 4-ureido-5-carboxyimidazole amide hydrolase expressed by Natronincola peptidivorans with accession number A0A1I0F426.

SEQ ID NO: 21 shows the amino acid sequence of 4-ureido-5-carboxyimidazole amide hydrolase expressed by [Clostridium] ultunense Esp with the accession number M1Z565.

SEQ ID NO: 22 shows the amino acid sequence of 4-ureido-5-carboxyimidazole amide hydrolase expressed by Bacillus firmus with the accession number A0A380XN14.

SEQ ID NO: 23 shows the amino acid sequence of 4-ureido-5-carboxyimidazole amide hydrolase expressed by Paenibacillus typhae with the accession number A0A1G9DH31.

SEQ ID NO: 24 shows the amino acid sequence of the 4-ureido-5-carboxyimidazole amide hydrolase expressed by Paenibacillus sp. FSL R7-0273 with the accession number A0A089KQZ0.

SEQ ID NO: 25 shows the amino acid sequence of 4-ureido-5-carboxyimidazole amide hydrolase expressed by Natribacillus halophilus with the accession number A0A1G8N0M9.

SEQ ID NO: 26 shows the amino acid sequence of the 4-ureido-5-carboxyimidazole amide hydrolase expressed by Caloramator australicus RC3 with the accession number I7K6S0.

SEQ ID NO: 27 shows the amino acid sequence of 4-ureido-5-carboxyimidazole amide hydrolase expressed by Bacillus sp. 7894-2 with the accession number A0A268IWI7.

SEQ ID NO: 28 shows the amino acid sequence of 4-ureido-5-carboxyimidazole amide hydrolase expressed by Paenibacillus sp. FSL R5-0912 with the accession number A0A089K3F8.

SEQ ID NO: 29 shows the amino acid sequence of 4-ureido-5-carboxyimidazole amide hydrolase expressed by Carboxydothermus hydrogenoformans (strain ATCC BAA-161/DSM 6008/Z-2901) with the accession number Q3AEA0.

SEQ ID NO: 30 shows the amino acid sequence of 4-ureido-5-carboxyimidazole amide hydrolase expressed by Bacillus sp. 3-2-2 with the accession number A0A429Y504.

SEQ ID NO: 31 shows the amino acid sequence of 4-ureido-5-carboxyimidazole amide hydrolase expressed by Bacillus sp. 2_A_57_CT2 with the accession number E5WNF5.

SEQ ID NO: 32 shows the amino acid sequence of 4-ureido-5-carboxyimidazole amide hydrolase expressed by Paraclostridium benzoelyticum with the accession number AOAOM3DKI8.

SEQ ID NO: 33 shows the amino acid sequence of 4-ureido-5-carboxyimidazole amide hydrolase expressed by Gottschalkia acidurici (strain ATCC7906/DSM 604/BCRC 14475/CIP 104303/NCIMB 10678/9a) with the accession number KOB1M2.

SEQ ID NO: 34 shows the amino acid sequence of 4-ureido-5-carboxyimidazole amide hydrolase expressed by Bacillus notoginsengisoli with the accession number A0A417YWC1.

SEQ ID NO: 35 shows the amino acid sequence of the 4-ureido-5-carboxyimidazole amide hydrolase expressed by Romboutsia lituseburensis DSM 797 with the accession number A0A1G9M563.

SEQ ID NO: 36 shows the amino acid sequence of 4-ureido-5-carboxyimidazole amide hydrolase expressed by Carboxydo thermuspertinax with the accession number A0A1L8CSS8.

SEQ ID NO: 37 shows the amino acid sequence of 4-ureido-5-carboxyimidazole amide hydrolase expressed by Hydrogenobacter hydrogenoformans DSM 2784 with the accession number A0A1G5S5M1.

SEQ ID NO: 38 shows the amino acid sequence of 4-ureido-5-carboxyimidazole amide hydrolase expressed by Soehngenia saccharolytica with the accession number A0A4T9ZU98.

SEQ ID NO: 39 shows the amino acid sequence of 4-ureido-5-carboxyimidazole amide hydrolase expressed by Tindallia calforniensis with the accession number A0A1H3Q6Y4.

SEQ ID NO: 40 shows the amino acid sequence of the 4-ureido-5-carboxyimidazole amide hydrolase expressed by Bacillus sp. OV194 with the accession number A0A1IlX4Y8.

SEQ ID NO: 41 shows the amino acid sequence of the 4-ureido-5-carboxyimidazole amide hydrolase expressed by Sulfobacillus benefaciens with the accession number A0A2T2XGJ2.

SEQ ID NO: 42 shows the amino acid sequence of 4-ureido-5-carboxyimidazole amide hydrolase expressed by Bacillus solani with the accession number A0A0Q3QV65.

SEQ ID NO: 43 shows the amino acid sequence of 4-ureido-5-carboxyimidazole amide hydrolase expressed by Clostridium sp. NCR with the accession number A0A099HWR1.

SEQ ID NO: 44 shows the amino acid sequence of 4-ureido-5-carboxyimidazole amide hydrolase expressed by Paenibacillus sp. FSL H8-0259 with the accession number A0A1R1CK04.

SEQ ID NO: 45 shows the amino acid sequence of the 4-ureido-5-carboxyimidazole amide hydrolase expressed by Caloramator mitchellensis with the accession number A0A0R3K3G8.

SEQ ID NO: 46 shows the amino acid sequence of the 4-ureido-5-carboxyimidazole amide hydrolase expressed by Sporosarcina globispora with the accession number A0A0M0GKI5.

SEQ ID NO: 47 shows the amino acid sequence of 4-ureido-5-carboxyimidazole amide hydrolase expressed by Paraclostridium bifermentans with the accession number A0A1X2JM05.

SEQ ID NO: 48 shows the amino acid sequence of the 4-ureido-5-carboxyimidazole amide hydrolase expressed by Proteiniborus sp. DW1 with the accession number A0A1M4MA16.

SEQ ID NO: 49 shows the amino acid sequence of the 4-ureido-5-carboxyimidazole amide hydrolase expressed by Maledivibacter halophilus with the accession number A0A1T5JUN3.

SEQ ID NO: 50 shows the amino acid sequence of 4-ureido-5-carboxyimidazole amide hydrolase expressed by Tepidimicrobium xylanilyticum with the accession number A0A1H2RJQ1.

SEQ ID NO: 51 shows the amino acid sequence of 4-ureido-5-carboxyimidazole amide hydrolase expressed by Tissierella sp. P1 with the accession number A0A265Q2F7.

SEQ ID NO: 52 shows the amino acid sequence of 4-ureido-5-carboxyimidazole amide hydrolase expressed by Paraclostridium bifermentans ATCC 638 with the accession number T4VR39.

SEQ ID NO: 53 shows the amino acid sequence of 4-ureido-5-carboxyimidazole amide hydrolase expressed by Andreesenia angusta with the accession number A0A1 S1V4I3.

SEQ ID NO: 54 shows the amino acid sequence of 4-ureido-5-carboxyimidazole amide hydrolase expressed by Tissierella praeacuta DSM 18095 with the accession number A0A1M4UHS4.

SEQ ID NO: 55 shows the amino acid sequence of the 4-ureido-5-carboxyimidazole amide hydrolase expressed by Ammoniphilus sp. CFH90114 with the accession number A0A4Q1SVQ3.

SEQ ID NO: 56 shows the amino acid sequence of 4-ureido-5-carboxyimidazole amide hydrolase expressed by Bacillus sp. c195 with the accession number A0A1I6C7D4.

SEQ ID NO: 57 shows the amino acid sequence of 4-ureido-5-carboxyimidazole amide hydrolase expressed by Marininema halotolerans with the accession number A0A1I6SUX1.

SEQ ID NO: 58 shows the amino acid sequence of 4-ureido-5-carboxyimidazole amide hydrolase expressed by Marininema mesophilum with the accession number A0A1H2QU42.

SEQ ID NO: 59 shows the amino acid sequence of 4-ureido-5-carboxyimidazole amide hydrolase expressed by Clostridiaceae bacterium with the accession number A0A3D2NTW4.

SEQ ID NO: 60 shows the amino acid sequence of 4-ureido-5-carboxyimidazole amide hydrolase expressed by Bacillus oceanisediminis with the accession number A0A2V2ZTK2.

SEQ ID NO: 61 shows the amino acid sequence of 4-ureido-5-carboxyimidazole amide hydrolase expressed in compost metagenome with the accession number A0A3R2KN38.

SEQ ID NO: 62 shows the amino acid sequence of 4-ureido-5-carboxyimidazole amide hydrolase expressed by Thermotalea metallivorans with the accession number A0A140L3I4.

SEQ ID NO: 63 shows the amino acid sequence of the 4-ureido-5-carboxyimidazole amide hydrolase expressed by Caloramator fervidus with the accession number A0A1H5RVB0.

SEQ ID NO: 64 shows the amino acid sequence of the 4-ureido-5-carboxyimidazole amide hydrolase expressed by Paludifilum halophilum with the accession number A0A235B9X9.

SEQ ID NO: 65 shows the amino acid sequence of 4-ureido-5-carboxyimidazole amide hydrolase expressed by Bacillus fortis with the accession number A0A443ILI9.

SEQ ID NO: 66 shows the amino acid sequence of the 4-ureido-5-carboxyimidazole amide hydrolase expressed by Paenibacillus sp. FSL R5-0490 with the accession number A0A1R1FF41.

SEQ ID NO: 67 shows the amino acid sequence of 4-ureido-5-carboxyimidazole amide hydrolase expressed in compost metagenome with the accession number A0A3R1V930.

SEQ ID NO: 68 shows the amino acid sequence of the 4-ureido-5-carboxyimidazole amide hydrolase expressed by Thermosyntropha lipolytica DSM 11003 with the accession number A0A1M5RCB4.

SEQ ID NO: 69 shows the amino acid sequence of 4-ureido-5-carboxyimidazole amide hydrolase expressed by Fictibacillus sp. S7 with the accession number A0A4Q2HVK6.

SEQ ID NO: 70 shows the amino acid sequence of 4-ureido-5-carboxyimidazole amide hydrolase expressed by Bacillus terrae with the accession number A0A429X1Q2.

SEQ ID NO: 71 shows the amino acid sequence of the 4-ureido-5-carboxyimidazole amide hydrolase expressed by Bacillus firmus DS1 with the accession number W7KSG1.

SEQ ID NO: 72 shows the amino acid sequence of 4-ureido-5-carboxyimidazole amide hydrolase expressed by Bacillus oceanisediminis 2691 with the accession number A0A160MH62.

SEQ ID NO: 73 shows the amino acid sequence of 4-ureido-5-carboxyimidazole amide hydrolase expressed by Tissierella creatinini with the accession number A0A4V5KXZ2.

SEQ ID NO: 74 shows the amino acid sequence of the 4-ureido-5-carboxyimidazole amide hydrolase expressed by Virgibacillus indicus with the accession number A0A265N915.

SEQ ID NO: 75 shows the amino acid sequence of 4-ureido-5-carboxyimidazole amide hydrolase expressed by Bacillus praedii with the accession number A0A4R1B0K9.

SEQ ID NO: 76 shows the amino acid sequence of 4-ureido-5-carboxyimidazole amide hydrolase expressed by Fictibacillus solisalsi with the accession number A0A1G9U716.

SEQ ID NO: 77 shows the amino acid sequence of the 4-ureido-5-carboxyimidazole amide hydrolase expressed by Acinetobacter sp. RIT592 with the accession number A0A369PBW3.

SEQ ID NO: 78 shows the amino acid sequence of 4-ureido-5-carboxyimidazole amide hydrolase expressed by Paenibacillus sp. IHB B 3415 with the accession number A0A0B2F366.

SEQ ID NO: 79 shows the amino acid sequence of 4-ureido-5-carboxyimidazole amide hydrolase expressed by Anaerovirgula multivorans with the accession number A0A239FUY4.

SEQ ID NO: 80 shows the amino acid sequence of 4-ureido-5-carboxyimidazole amide hydrolase expressed by Tissierella praeacuta with the accession number A0A3F3S6Q3.

SEQ ID NO: 81 shows the amino acid sequence of 4-ureido-5-carboxyimidazole amide hydrolase expressed by Alkaliphilus sp. with the accession number A0A2G2MLG7.

SEQ ID NO: 82 shows the amino acid sequence of 4-ureido-5-carboxyimidazole amide hydrolase expressed by Paenibacillus donghaensis with the accession number A0A2Z2KPK9.

SEQ ID NO: 83 shows the amino acid sequence of the 4-ureido-5-carboxyimidazole amide hydrolase expressed by Ammoniphilus sp. CFH 90114 with the accession number A0A4Q1SUH8.

SEQ ID NO: 84 shows the amino acid sequence of 4-ureido-5-carboxyimidazole amide hydrolase expressed by Paenibacillus borealis with the accession number A0A089LLJ5.

SEQ ID NO: 85 shows the amino acid sequence of the 4-ureido-5-carboxyimidazole amide hydrolase expressed by Thermohalobacter berrensis with the accession number A0A419T2D3.

SEQ ID NO: 86 shows the amino acid sequence of the 4-ureido-5-carboxyimidazole amide hydrolase expressed by Tindallia magadiensis with the accession number A0A1I3ETE5.

SEQ ID NO: 87 shows the amino acid sequence of the 4-ureido-5-carboxyimidazole amide hydrolase expressed by Ornithinibacillus halophilus with the accession number A0A1M5FPE9.

SEQ ID NO: 88 shows the amino acid sequence of the 4-ureido-5-carboxyimidazole amide hydrolase expressed by Paenibacillus sp. NFR01 with the accession number A0A1I0KBZ0.

SEQ ID NO: 89 shows the amino acid sequence of 4-ureido-5-carboxyimidazole amide hydrolase expressed by Bacillus oceanisediminis with the accession number A0A4R8GJZ7.

SEQ ID NO: 90 shows the amino acid sequence of the 4-ureido-5-carboxyimidazole amide hydrolase expressed by Soehngenia sp. 1933P with the accession number A0A4Z0D772.

SEQ ID NO: 91 shows the amino acid sequence of 4-ureido-5-carboxyimidazole amide hydrolase expressed by Soehngenia saccharolytica with the accession number A0A4V5LAZ6.

SEQ ID NO: 92 shows the amino acid sequence of 4-ureido-5-carboxyimidazole amide hydrolase expressed by Proteiniborus ethanoligenes with the accession number A0A1H3NHT6.

SEQ ID NO: 93 shows the amino acid sequence of 4-ureido-5-carboxyimidazole amide hydrolase expressed by Anaeromicrobium sediminis with the accession number A0A267MJY2.

SEQ ID NO: 94 shows the amino acid sequence of 4-ureido-5-carboxyimidazole amide hydrolase expressed by Bacillus freudenreichii with the accession number A0A3S4Q236.

SEQ ID NO: 95 shows the amino acid sequence of the 4-ureido-5-carboxyimidazole amide hydrolase expressed by Alkaliphilus metalliredigens (strain QYMF) with the accession number A6TWT6.

SEQ ID NO: 96 shows the amino acid sequence of 4-ureido-5-carboxyimidazole amide hydrolase expressed by Thermoflavimicrobium sp. FBKL4.011 with the accession number A0A364K2P5.

SEQ ID NO: 97 shows the amino acid sequence of 4-ureido-5-carboxyimidazole amide hydrolase expressed by Bacteroidetes bacterium 4572_77 with the accession number A0A256WHJ3.

SEQ ID NO: 98 shows the amino acid sequence of the 4-ureido-5-carboxyimidazole amide hydrolase expressed by Ammoniphilus sp. CFH 90114 with the accession number A0A4Q1SVB7.

SEQ ID NO: 99 shows the amino acid sequence of 4-ureido-5-carboxyimidazole amide hydrolase expressed by Gottschalkia purinilytica with the accession number A0A0L0W6E3.

SEQ ID NO: 100 shows the amino acid sequence of 4-ureido-5-carboxyimidazole amide hydrolase expressed by Bacillus bacterium with the accession number A0A3D0EAH7.

SEQ ID NO: 101 shows the amino acid sequence of 4-ureido-5-carboxyimidazole amide hydrolase expressed by Paenibacillus sp. with the accession number A0A1J7J0P5.

SEQ ID NO: 102 shows the amino acid sequence of 4-ureido-5-carboxyimidazole amide hydrolase expressed by Paraclostridium bifermentans with the accession number T4VC62.

SEQ ID NO: 103 shows the amino acid sequence of xanthine amide hydrolase expressed by Bacillus firmus (CGMCC 1.2010).

SEQ ID NO: 104 shows the nucleotide sequence of the upstream primer used for PCR amplification of a nucleic acid molecule encoding xanthine amide hydrolase.

SEQ ID NO: 105 shows the nucleotide sequence of the downstream primer used for PCR amplification of a nucleic acid molecule encoding xanthine amide hydrolase.

SEQ ID NO: 106 shows the amino acid sequence of the xanthine amide hydrolase expressed by Clostridium purinilyticum with the accession number A0A0L0W692.

SEQ ID NO: 107 shows the amino acid sequence of the xanthine amide hydrolase expressed by Thermoflavimicrobium sp. FBKL4.011 with the accession number A0A364K317.

SEQ ID NO: 108 shows the amino acid sequence of the xanthine amide hydrolase expressed by Marininema halotolerans with the accession number A0A1I6SUY8.

SEQ ID NO: 109 shows the amino acid sequence of the xanthine amide hydrolase expressed by Clostridiaceae bacterium with the accession number A0A3D2NSM2.

SEQ ID NO: 110 shows the amino acid sequence of the xanthine amide hydrolase expressed by Bacillus sp. 2_A_57_CT2 with the accession number E5WNF6.

SEQ ID NO: 111 shows the amino acid sequence of the xanthine amide hydrolase expressed by Paenibacillus sp. FSL H8-0259 with the accession number A0A1R1CK18.

SEQ ID NO: 112 shows the amino acid sequence of the xanthine amide hydrolase expressed by Thermoflavimicrobium dichotomicum with the accession number A0A1I3U261.

SEQ ID NO: 113 shows the amino acid sequence of the xanthine amide hydrolase expressed by Fictibacillus enclensis with the accession number A0A0V8JCN2.

SEQ ID NO: 114 shows the amino acid sequence of the xanthine amide hydrolase expressed by Marininema mesophilum with the accession number A0A1H2QVP9.

SEQ ID NO: 115 shows the amino acid sequence of the xanthine amide hydrolase expressed by Paenibacillus sp. FSL R5-0912 with the accession number A0A089K5P4.

SEQ ID NO: 116 shows the amino acid sequence of the xanthine amide hydrolase expressed by Paenibacillus typhae with the accession number A0A1G9DGW2.

SEQ ID NO: 117 shows the amino acid sequence of the xanthine amide hydrolase expressed by Tissierella praeacuta DSM 18095 with the accession number A0A1M4UHZ6.

SEQ ID NO: 118 shows the amino acid sequence of the xanthine amide hydrolase expressed by Bacillus sp. c195 with the accession number A0A1I6C7J4.

SEQ ID NO: 119 shows the amino acid sequence of the xanthine amide hydrolase expressed by Bradyrhizobium japonicum with the accession number A0A0A3XEE6.

SEQ ID NO: 120 shows the amino acid sequence of the xanthine amide hydrolase expressed by Bacillus firmus with the accession number A0A380XTT6.

SEQ ID NO: 121 shows the amino acid sequence of xanthine amide hydrolase expressed by Acidaminobacter hydrogenoformans DSM 2784 with the accession number A0A1G5S5P6.

SEQ ID NO: 122 shows the amino acid sequence of the xanthine amide hydrolase expressed by Caloranaerobacter sp. TR13 with the accession number A0A0P8Z9T7.

SEQ ID NO: 123 shows the amino acid sequence of the xanthine amide hydrolase expressed by Tissierella sp. P1 with the accession number A0A265Q2B2.

SEQ ID NO: 124 shows the amino acid sequence of the xanthine amide hydrolase expressed by Bacillus sp. 3-2-2 with the accession number A0A429Y566.

SEQ ID NO: 125 shows the amino acid sequence of the xanthine amide hydrolase expressed by Paenibacillus donghaensis with the accession number A0A2Z2KEH1.

SEQ ID NO: 126 shows the amino acid sequence of the xanthine amide hydrolase expressed by Gottschalkia acidurici (strain ATCC 7906/DSM 604/BCRC 14475/CIP 104303/NCIMB 10678/9a) with the accession number K0AWA5.

SEQ ID NO: 127 shows the amino acid sequence of the xanthine amide hydrolase expressed by Bacillus fortis with the accession number A0A443IKX3.

SEQ ID NO: 128 shows the amino acid sequence of the xanthine amide hydrolase expressed by Bacillus oceanisediminis with the accession number A0A2V2ZNB0.

SEQ ID NO: 129 shows the amino acid sequence of the xanthine amide hydrolase expressed by Virgibacillus profundi with the accession number A0A2A2IEE5.

SEQ ID NO: 130 shows the amino acid sequence of the xanthine amide hydrolase expressed by Paenibacillus sp. FSL R7-0331 with the accession number A0A089MDW3.

SEQ ID NO: 131 shows the amino acid sequence of the xanthine amide hydrolase expressed by Bacillus sp. FJAT-21945 with the accession number A0A0M0X8C9.

SEQ ID NO: 132 shows the amino acid sequence of xanthine amide hydrolase expressed by Tissierella praeacuta with the accession number A0A3F3S6I2.

SEQ ID NO: 133 shows the amino acid sequence of the xanthine amide hydrolase expressed by Bacillus oceanisediminis with the accession number A0A1S1YDG4.

SEQ ID NO: 134 shows the amino acid sequence of the xanthine amide hydrolase expressed by Bacillus sp. OV194 with the accession number A0A1I1X526.

SEQ ID NO: 135 shows the amino acid sequence of the xanthine amide hydrolase expressed by Anaeromicrobium sediminis with the accession number A0A267MJF0.

SEQ ID NO: 136 shows the amino acid sequence of the xanthine amide hydrolase expressed by Thermosyntropha lipolytica DSM 11003 with the accession number A0A1M5RDL0.

SEQ ID NO: 137 shows the amino acid sequence of the xanthine amide hydrolase expressed by Alkaliphilus peptidifermentans DSM 18978 with the accession number A0A1G5KXH0.

SEQ ID NO: 138 shows the amino acid sequence of the xanthine amide hydrolase expressed by Aneurinibacillus migulanus with the accession number A0A1G8Q7H9.

SEQ ID NO: 139 shows the amino acid sequence of the xanthine amide hydrolase expressed by Paenibacillus sp. IHB B 3415 with the accession number A0A0B2F3Z2.

SEQ ID NO: 140 shows the amino acid sequence of the xanthine amide hydrolase expressed by Marinisporobacter balticus with the accession number A0A4R2KME8.

SEQ ID NO: 141 shows the amino acid sequence of the xanthine amide hydrolase expressed by Bacillus terrae with the accession number A0A429X0W8.

SEQ ID NO: 142 shows the amino acid sequence of the xanthine amide hydrolase expressed by Tindallia californiensis with the accession number A0A1H3Q6T8.

SEQ ID NO: 143 shows the amino acid sequence of the xanthine amide hydrolase expressed by Clostridium acidurici (strain ATCC 7906/DSM 604/BCRC 14475/CIP 104303/NCIMB 10678/9a) with the accession number KOB360.

SEQ ID NO: 144 shows the amino acid sequence of the xanthine amide hydrolase expressed by Romboutsia lituseburensis DSM 797 with the accession number A0A1G9M635.

SEQ ID NO: 145 shows the amino acid sequence of the xanthine amide hydrolase expressed by Paraclostridium bifermentans ATCC 19299 with the accession number T4V5T0.

SEQ ID NO: 146 shows the amino acid sequence of the xanthine amide hydrolase expressed by Bacillus praedii with the accession number A0A4R1ATL6.

SEQ ID NO: 147 shows the amino acid sequence of the xanthine amide hydrolase expressed by Carbydothermus islandicus with the accession number A0A1L8D5S0.

SEQ ID NO: 148 shows the amino acid sequence of the xanthine amide hydrolase expressed by Caloramator australicus RC3 with the accession number I7KTT3.

SEQ ID NO: 149 shows the amino acid sequence of the xanthine amide hydrolase expressed by Paraclostridium bifermentans ATCC 638 with the accession number T4VRB8.

SEQ ID NO: 150 shows the amino acid sequence of the xanthine amide hydrolase expressed by Tepidimicrobium xylanilyticum with the accession number A0A1H2RLU1.

SEQ ID NO: 151 shows the amino acid sequence of the xanthine amide hydrolase expressed by Aneurinibacillus migulanus with the accession number A0A0K2WJ73.

SEQ ID NO: 152 shows the amino acid sequence of the xanthine amide hydrolase expressed by Bacillus bacterium with the accession number A0A3D0EBN7.

SEQ ID NO: 153 shows the amino acid sequence of the xanthine amide hydrolase expressed by Bacillus notoginsengisoli with the accession number A0A417YW08.

SEQ ID NO: 154 shows the amino acid sequence of the xanthine amide hydrolase expressed by Bacillus oceanisediminis 2691 with the accession number A0A160MBB4.

SEQ ID NO: 155 shows the amino acid sequence of the xanthine amide hydrolase expressed by Paenibacillus sp. FSL R7-0273 with the accession number A0A089LXU3.

SEQ ID NO: 156 shows the amino acid sequence of the xanthine amide hydrolase expressed by [Clostridium] ultunense Esp with the accession number M1ZGS5.

SEQ ID NO: 157 shows the amino acid sequence of the xanthine amide hydrolase expressed by Bacillus freudenreichii with the accession number A0A448FF64.

SEQ ID NO: 158 shows the amino acid sequence of the xanthine amide hydrolase expressed by Caloramator fervidus with the accession number A0A1H5RUL9.

SEQ ID NO: 159 shows the amino acid sequence of the xanthine amide hydrolase expressed by Bacillus oceanisediminis with the accession number A0A4R8GVS7.

SEQ ID NO: 160 shows the amino acid sequence of the xanthine amide hydrolase expressed by Soehngenia saccharolytica with the accession number A0A4T9ZWH0.

SEQ ID NO: 161 shows the amino acid sequence of the xanthine amide hydrolase expressed by Bacillus firmus DS1 with the accession number W7LB72.

SEQ ID NO: 162 shows the amino acid sequence of the xanthine amide hydrolase expressed by Thermotalea metallivorans with the accession number A0A140L3I5.

SEQ ID NO: 163 shows the amino acid sequence of the xanthine amide hydrolase expressed by Ornithinibacillus halophilus with the accession number A0A1M5FNK3.

SEQ ID NO: 164 shows the amino acid sequence of the xanthine amide hydrolase expressed by Thermosyntropha lipolytica DSM 11003 with the accession number A0A1M5LQE8.

SEQ ID NO: 165 shows the amino acid sequence of the xanthine amide hydrolase expressed by Fictibacillus sp. S7 with the accession number A0A4Q2HRQ1.

SEQ ID NO: 166 shows the amino acid sequence of the xanthine amide hydrolase expressed by Bacillus mesonae with the accession number A0A3Q9QZ31.

SEQ ID NO: 167 shows the amino acid sequence of the xanthine amide hydrolase expressed by Clostridium purinilyticum with the accession number A0A0L0W688.

SEQ ID NO: 168 shows the amino acid sequence of the xanthine amide hydrolase expressed by Tindallia magadiensis with the accession number A0A1I3ETA9.

SEQ ID NO: 169 shows the amino acid sequence of the xanthine amide hydrolase expressed by Paenibacillus borealis with the accession number A0A089LHF8.

SEQ ID NO: 170 shows the amino acid sequence of the xanthine amide hydrolase expressed by Paraclostridium benzoelyticum with the accession number A0A0M3DN30.

SEQ ID NO: 171 shows the amino acid sequence of the xanthine amide hydrolase expressed by Bacillus solani with the accession number A0A0Q3QMR7.

SEQ ID NO: 172 shows the amino acid sequence of the xanthine amide hydrolase expressed by Thermohalobacter berrensis with the accession number A0A419T2I0.

SEQ ID NO: 173 shows the amino acid sequence of the xanthine amide hydrolase expressed by Acinetobacter sp. RIT592 with the accession number A0A369PBX6.

SEQ ID NO: 174 shows the amino acid sequence of the xanthine amide hydrolase expressed by Maledivibacter halophilus with the accession number A0A1T5JUM9.

SEQ ID NO: 175 shows the amino acid sequence of the xanthine amide hydrolase expressed by Tissierella creatinini with the accession number A0A4T9WHZ3.

SEQ ID NO: 176 shows the amino acid sequence of the xanthine amide hydrolase expressed by compost metagenome with the accession number A0A3R1HSK2.

SEQ ID NO: 177 shows the amino acid sequence of the xanthine amide hydrolase expressed by Natronincola peptidivorans with the accession number A0A1I0F3P2.

SEQ ID NO: 178 shows the amino acid sequence of the xanthine amide hydrolase expressed by Bacillus firmus with the accession number A0A0J5YTU9.

SEQ ID NO: 179 shows the amino acid sequence of the xanthine amide hydrolase expressed by Anaerovirgula multivorans with the accession number A0A239FV01.

SEQ ID NO: 180 shows the amino acid sequence of the xanthine amide hydrolase expressed by Carbydothermus hydrogenoformans (strain ATCC BAA-161/DSM 6008/Z-2901) with the accession number Q3AEA1.

SEQ ID NO: 181 shows the amino acid sequence of xanthine amide hydrolase expressed by Sporanaerobacter acetigenes DSM 13106 with the accession number A0A1M5YST8.

SEQ ID NO: 182 shows the amino acid sequence of the xanthine amide hydrolase expressed by Proteiniborus sp. DW1 with the accession number A0A1M4MA63.

SEQ ID NO: 183 shows the amino acid sequence of the xanthine amide hydrolase expressed by Bacillus sp. 7894-2 with the accession number A0A268IWM5.

SEQ ID NO: 184 shows the amino acid sequence of the xanthine amide hydrolase expressed by [Clostridium] ultunense Esp with the accession number A0A1M4PLJ1.

SEQ ID NO: 185 shows the amino acid sequence of the xanthine amide hydrolase expressed by Virgibacillus indicus with the accession number A0A265N7L4.

SEQ ID NO: 186 shows the amino acid sequence of the xanthine amide hydrolase expressed by Paenibacillus sp. NFR01 with the accession number A0A1I0KAI3.

SEQ ID NO: 187 shows the amino acid sequence of the xanthine amide hydrolase expressed by Andreesenia angus with the accession number A0A1S1V3Q5.

SEQ ID NO: 188 shows the amino acid sequence of the xanthine amide hydrolase expressed by Paenibacillus sp. DMB5 with the accession number A0A117T0P2.

SEQ ID NO: 189 shows the amino acid sequence of the xanthine amide hydrolase expressed by Paludifilum halophilum with the accession number A0A235B9X8.

SEQ ID NO: 190 shows the amino acid sequence of the xanthine amide hydrolase expressed by Proteiniborus ethanoligenes with the accession number A0A1H3NJW1.

SEQ ID NO: 191 shows the amino acid sequence of the xanthine amide hydrolase expressed by Alkaliphilus metalliredigens (strain QYMF) with the accession number A6TWT7.

SEQ ID NO: 192 shows the amino acid sequence of the xanthine amide hydrolase expressed by Fictibacillus solisalsi with the accession number A0A1G9U6Z5.

SEQ ID NO: 193 shows the amino acid sequence of the xanthine amide hydrolase expressed by Sporosarcina globispora with the accession number A0A0M0GGH4.

SEQ ID NO: 194 shows the amino acid sequence of the xanthine amide hydrolase expressed by Bacillus firmus with the accession number A0A366K523.

SEQ ID NO: 195 shows the amino acid sequence of the xanthine amide hydrolase expressed by Paenibacillus sp. FSL R5-0490 with the accession number A0A1R1FFH4.

SEQ ID NO: 196 shows the amino acid sequence of the xanthine amide hydrolase expressed by Alkaliphilus sp. with the accession number A0A2G2MLE4.

SEQ ID NO: 197 shows the amino acid sequence of the xanthine amide hydrolase expressed by Alkaliphilus oremlandii strain OhILAs with the accession number A8MLA7.

SEQ ID NO: 198 shows the amino acid sequence of the xanthine amide hydrolase expressed by Caloramator mitchellensis with the accession number A0A0R3JVG6.

SEQ ID NO: 199 shows the amino acid sequence of the xanthine amide hydrolase expressed by Clostridium cylindrosporum DSM 605 with the accession number A0A0J8G334.

SEQ ID NO: 200 shows the amino acid sequence of the xanthine amide hydrolase expressed by the Ammoniphilus sp. CFH 90114 with the accession number A0A4Q1SWC2.

SEQ ID NO: 201 shows the amino acid sequence of the xanthine amide hydrolase expressed by Carbydothermus pertinax with the accession number A0A1L8CSM0.

SEQ ID NO: 202 shows the amino acid sequence of the xanthine amide hydrolase expressed by Soehngenia sp. 1933P with the accession number A0A4Z0D783.

SEQ ID NO: 203 shows the amino acid sequence of the xanthine amide hydrolase expressed by Soehngenia saccharolytica with the accession number A0A4T9ZOT2.

SEQ ID NO: 204 shows the amino acid sequence of the xanthine amide hydrolase expressed by Natribacillus halophilus with the accession number A0A1G8N2H8.

SEQ ID NO: 205 shows the amino acid sequence of the xanthine amide hydrolase expressed by compost metagenome with the accession number A0A403WBU6.

SEQ ID NO: 206 shows the amino acid sequence of the xanthine amide hydrolase expressed by compost metagenome with the accession number A0A3R4A6V9

SEQ ID NO: 207 shows the inserted sequence of the nucleic acid molecule containing the promoter.

SEQ ID NO: 208 shows the inserted sequence of the nucleic acid molecule encoding xanthine amide hydrolase and 4-ureido-5-carboxyimidazole amide hydrolase.

### DETAILED DESCRIPTION OF THE INVENTION

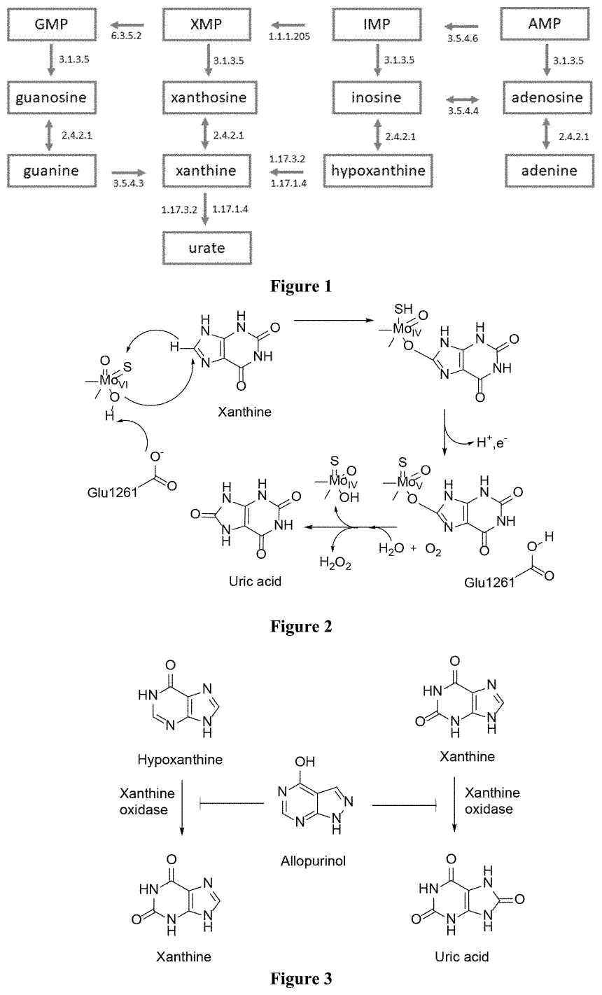

FIG. 1 shows the metabolic pathways of various purines in the human body. The final product of various purine metabolism is urate, and the deposition of urate in joints and kidneys is the cause of gout. FIG. 2 shows the process by which the aerobic protein xanthine oxidase catalyzes the degradation of xanthine to produce uric acid. The gout drug allopurinol inhibits the production of uric acid by inhibiting xanthine oxidase, thereby treating gout (FIG. 3). GDVOGELS et al. (Degradation of Purines and Pyrimidines by Microorganisms, Bacteriological Reviews, June 1976, Vol. 40, No. 2, p. 403-468) reported that xanthine can be degraded to iminomethylglycine by Clostridium cylindrosporum under anaerobic conditions.

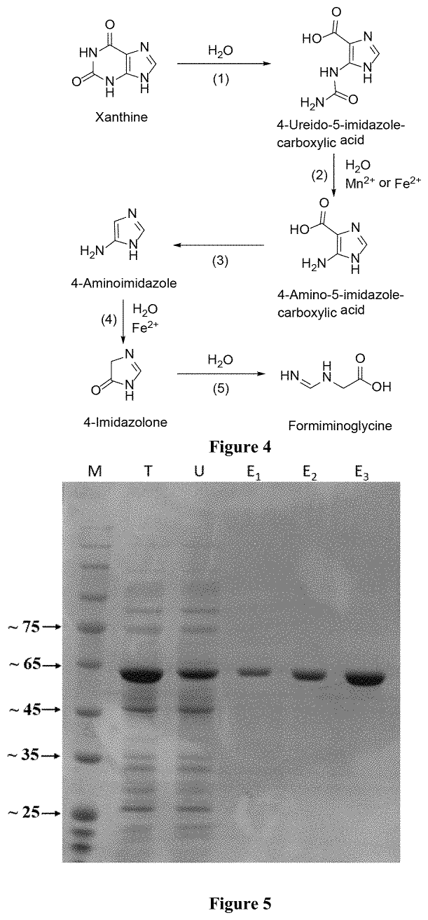

FIG. 4 shows the reaction formulas of xanthine amide hydrolase degrading xanthine under anaerobic conditions and 4-ureido-5-carboxyimidazole amide hydrolase degrading 4-ureido-5-carboxyimidazole under anaerobic conditions. The inventor of the present application realizes that purine degradation under anaerobic conditions is of great significance, because the human intestinal tract is an anaerobic environment, if it can precede the absorption of purines in the intestinal tract and degrade purines in the intestinal tract, this is important for prevention, intervention, alleviation and/or treatment of gout, this is a new idea. The inventor of the present application successfully identified xanthine amide hydrolase that degrades xanthine under anaerobic conditions after doing a lot of work using bioinformatics methods.

Unless otherwise specified, the terms used in this application have the meanings commonly understood by those skilled in the art.

### Definition

Unless otherwise indicated, nucleic acids are written from left to right in the 5′ to 3′ direction, respectively; amino acid sequences are written from left to right in the amino to carboxy direction, respectively. The number range includes the number that defines the range. Amino acids can be represented herein by their commonly known three-letter symbols or the one-letter symbols recommended by the IUPAC-IUB Biochemical Nomenclature Committee. Likewise, the commonly accepted one-letter codes can be used to represent nucleotides. Refer to the specification as a whole to more fully define the terms defined above.

“Polypeptide” and “protein” in this application are used interchangeably herein and refer to polymers of amino acid residues and their variants and synthetic and naturally-occurring analogs. Therefore, these terms apply to naturally-occurring amino acid polymers and their naturally-occurring chemical derivatives, and to non-naturally-occurring amino acids in which one or more amino acid residues are synthetic (such as chemical analogs of the corresponding naturally-occurring amino acids) amino acid polymer. Such derivatives include, for example, post-translational modification and degradation products, including phosphorylated, glycosylated, oxidized, isomerized, carboxylated, and deaminated variants of polypeptide fragments.

The term “enzyme active center” as used herein refers to the part of the enzyme molecule that can directly bind to the substrate molecule and catalyze the chemical reaction of the substrate, and this part becomes the active center of the enzyme. It is generally believed that the active center is mainly composed of two functional sites: the first is the catalytic site, where the bond of the substrate is broken or a new bond is formed to cause certain chemical changes; the second is the binding site, which is the substrate binding to the enzyme molecule. The functional part is composed of a few amino acid residues or some groups on these residues that are relatively close in the three-dimensional structure of the enzyme molecule. They may be far apart in the primary structure, or even located on different peptide chains, but close to each other in spatial conformation through the winding and folding of the peptide chain; for enzymes that require coenzymes, coenzyme molecules (such as metal ions Zn²⁺ and/or Mn²⁺) or a certain part of the structure of the coenzyme molecule is also a part of the function site.

The term “amino acid” as used herein refers to a compound in which a hydrogen atom on a carbon atom of a carboxylic acid is replaced by an amino group, and the amino acid molecule contains two functional groups: an amino group and a carboxyl group. It includes naturally occurring and non-naturally occurring amino acids as well as amino acid analogs and mimetics. Naturally-occurring amino acids include 20 kinds of (L)-amino acids used in protein biosynthesis and other amino acids, such as 4-hydroxyproline, hydroxylysine, carboxylated lysine, catenin, isocatenin, homocysteine, citrulline and ornithine. Non-naturally occurring amino acids include, for example, (D)-amino acids, norleucine, norvaline, p-fluorophenylalanine, ethylthioine, etc., which are known to those skilled in the art. Amino acid analogs include modified forms of naturally occurring and non-naturally occurring amino acids. Such modifications may include, for example, substitution of chemical groups and moieties on amino acids, or derivatization of amino acids. Amino acid mimetics include, for example, organic structures that exhibit functionally similar properties, such as the charge and charge space properties of amino acids. For example, an organic structure that mimics arginine (Arg or R) has a positively charged moiety that is located in a similar molecular space and has the same degree of mobility as the e-amino group of the side chain of a naturally occurring Arg amino acid. Mimics also include constrained structures to maintain optimal space and charge interactions of amino acids or amino acid functional groups. Those skilled in the art can determine what structures constitute functionally equivalent amino acid analogs and amino acid mimetics.

The term “isoenzyme” as used herein refers to enzymes that catalyze the same reaction in an organism but have different molecular structures.

The term “nucleic acid” as used herein refers to mRNA, RNA, cRNA, cDNA or DNA, including single-stranded and double-stranded forms of DNA. The term generally refers to a polymeric form of nucleotides with a length of at least 10 bases, which are ribonucleotides or deoxynucleotides or a modified form of any type of nucleotide.

As used herein, the term “encoding” when used in the context of a specific nucleic acid means that the nucleic acid contains the necessary information to direct the translation of the nucleotide sequence into a specific protein. The use of codons represents information about the encoded protein. A nucleic acid encoding a protein may comprise untranslated sequences (e.g., introns) located within the translation region of the nucleic acid or may lack such intervening untranslated sequences (e.g., as in cDNA).

As used herein, a “full-length sequence” related to a specific polynucleotide or a protein encoded by it refers to the entire nucleic acid sequence or the entire amino acid sequence having a natural (non-synthetic) endogenous sequence. The full-length polynucleotide encodes the full-length, catalytically active form of the specific protein.

The term “isolated” as used herein refers to a polypeptide or nucleic acid or a biologically active portion thereof, which is substantially or essentially free of those normally accompanied or reacted with the protein or nucleic acid components as found in its naturally occurring environment. Therefore, when the isolated polypeptide A or nucleic acid is produced by recombinant technology, the isolated polypeptide A or nucleic acid is substantially free of other cellular substances or culture media, or when the isolated polypeptide A or nucleic acid is chemically synthesized, it is substantially free of chemical precursors or other chemicals.

The term “expression vector” as used herein is a recombinantly or synthetically produced nucleic acid construct that has a series of specific nucleic acid elements that allow specific nucleic acid to be transcribed in a host cell.

The term “host cell” as used herein refers to a cell that receives a foreign gene in transformation and transduction (infection). The host cell may be a eukaryotic cell such as a yeast cell or a prokaryotic cell such as E. coli.

### Specific Implementation Plan

In the first aspect, the present application provides polypeptide A, which comprises the amino acid sequence shown in SEQ ID NO:1 or a functional variant thereof, wherein the functional variant has 4-ureido-5-carboxyimidazole amide hydrolase activity.

In some embodiments of the first aspect, the polypeptide A has a catalytic site A defined as follows in spatial conformation:

The catalytic site A includes catalytic triads close to each other in spatial conformation.

In some embodiments of the first aspect, the catalytic triad is composed of C134, D10, and K101 with reference to SEQ ID NO:1.

In some embodiments of the first aspect, the amino acid residues C134 (affinity attack), D10 and K101 of SEQ ID NO:1 constitute a catalytic triad similar to a proteolytic enzyme.

In some embodiments of the first aspect, the catalytic site A further comprises a divalent metal ion.

In some embodiments of the first aspect, the catalytic site A further includes one divalent metal ion, such as Zn²⁺ or Mn²⁺.

In some embodiments of the first aspect, the catalytic site A further comprises four amino acid residues coordinated with the metal ion, such as H61, H74, E59 and D67.

The polypeptide A also includes a binding site A with the following definition in the spatial conformation: the binding site A includes the amino acid residues F15, R71, V129, H104 and W130 of SEQ ID NO:1 that are close to each other in the spatial conformation.

In some embodiments of the first aspect, H104 forms a hydrogen bond with the 5-carboxyl group of the substrate 4-ureido-5-carboxyimidazole.

In some embodiments of the first aspect, wherein the distance between the catalytic site A and the binding site A is no more than 5 angstroms

In some embodiments of the first aspect, wherein the functional variant is a natural isoenzyme of the amino acid sequence shown in SEQ ID NO:1.

In some embodiments of the first aspect, the functional variant is the natural isoenzyme of the amino acid sequence shown in SEQ ID NO:1 from: Clostridium cylindrosporum, Sporanaerobacter acetigenes, Thermosyntropha lipolytica, Aneurinibacillus migulanus, Alkaliphilus oremlandii, Fictibacillus enclensis, Bacillus sp., Bacillus mesonae, Bacillus oceanisediminis, Bacillus firmus, Marinisporobacter balticus, Alkaliphilus peptidifermentans, Thermoflavimicrobium dichotomicum, Paenibacillus sp., Caloranaerobacter sp., Virgibacillus profundi, Carboxydothermus islandicus, Natronincola peptidivorans, Natronincola peptidivorans, Paenibacillus typhae, Natribacillus halophilus, Caloramator australicus, Carbydothermus hydrogenoformans, Paraclostridium benzoelyticum, Gottschalkia acidurici, Bacillus notoginsengisoli, Romboutsia lituseburensis, Carboxydothermus pertinax, Acidaminobacter hydrogenoformans, Soehngenia saccharolytica, Tindallia californiensis, Sulfobacillus benefaciens, Bacillus solani, Clostridium sp., Caloramator mitchellensis, Sporosarcina globispora, Paraclostridium bifermentans, Proteiniborus sp., Maledivibacter halophilus, Tepidimicrobium xylanilyticum, Tissierella sp., Andreesenia angusta, Tissierella praeacuta, Ammoniphilus sp., Marininema halotolerans, Marininema mesophilum, Clostridiaceae bacterium, compost metagenome, Thermotalea metallivorans, Caloramator fervidus, Paludifilum halophilum, Bacillus fortis, Fictibacillus sp., Bacillus terrae, Tissierella creatinini, Virgibacillus indicus, Bacillus praedii, Fictibacillus solisalsi, Acinetobacter sp., Anaerovirgula multivorans, Alkaliphilus sp., Paenibacillus donghaensis, Paenibacillus borealis, Thermohalobacter berrensis, Tindallia magadiensis, Ornithinibacillus halophilus, Soehngenia sp., Proteiniborus ethanoligenes, Anaeromicrobium sediminis, Bacillus freudenreichii, Alkaliphilus metalliredigens, Thermoflavimicrobium sp., Bacteroidetes bacterium, Gottschalkia purinilytica, Bacillus bacterium and Paraclostridium bifermentans.

In some specific embodiments of the first aspect, the functional variant is the natural isoenzyme of the amino acid sequence shown in SEQ ID NO: 1 comprising the amino acid sequence shown in any one of SEQ ID NO: 2-102.

In some embodiments of the first aspect, wherein the functional variant is resulted from the insertion, substitution and/or deletion of one or more amino acids on the basis of the amino acid sequence shown in SEQ ID NO:1 or its natural isoenzyme.

In some embodiments of the first aspect, the insertion, substitution and/or deletion does not occur at the catalytic site A.

In some embodiments of the first aspect, the insertion, substitution and/or deletion does not occur at the binding site A.

In some embodiments of the first aspect, the insertion, substitution and/or deletion does not occur at the catalytic site A and the binding site A.

In some embodiments of the first aspect, the number of amino acid insertions, substitutions and/or deletions is 1-30, preferably 1-20, more preferably 1-10, wherein the obtained functional variant is basically remained unchanged the activity of 4-ureido-5-carboxyimidazole amide hydrolase.

In some embodiments of the first aspect, the functional variant differs from the amino acid sequence shown in SEQ ID NO:1 by about 1, 2, 3, 4, 5, 6, 7, 8, 9, or 10 amino acids insertions, substitutions and/or deletions.

In some embodiments of the first aspect, the functional variant differs from the amino acid sequence shown in SEQ ID NO:1 by about 1, 2, 3, 4 or 5 amino acid insertions, substitutions and/or deletions.

Certain amino acid substitutions called “conservative amino acid substitutions” can occur frequently in proteins without changing the conformation or function of the protein. This is an established rule in protein chemistry.

Conservative amino acid substitutions in this application include, but are not limited to, substitute any other of these aliphatic amino acids with any one of glycine (G), alanine (A), isoleucine (I), valine (V) and leucine (L); use serine (S) instead of threonine (T), and vice versa; use aspartate (D) instead of glutamate (E), and vice versa; use glutamine (Q) replace asparagine (N) and vice versa; replace arginine (R) with lysine (K) and vice versa; use phenylalanine (F), tyrosine (Y) and Tryptophan (W) substituting any other of these aromatic amino acids; and methionine (M) is substituted for cysteine (C), and vice versa. Other substitutions can also be considered conservative, depending on the specific amino acid environment and its role in the three-dimensional structure of the protein. For example, glycine (G) and alanine (A) are often interchangeable, just as alanine (A) and valine (V) are interchangeable. The relatively hydrophobic methionine (M) can often be interchanged with leucine and isoleucine, and sometimes with valine. Lysine (K) and arginine (R) are often interchanged in the following positions: the important feature of amino acid residues is their charge, and the difference in pK of these two amino acid residues is not obvious. Under certain circumstances, there are still other changes that can be considered “conservative” (see, for example, BIOCHEMISTRY at pp. 13-15, 2^{nd} ed. Lubert Stryer ed. (Stanford University); Henikoff et al. Proc. Nat'l Acad. Sci.

USA (1992) 89: 10915-10919; Lei et al., J. Biol. Chem. (1995) 270(20): 11882-11886).

Hereinafter, the amino acid residues are classified according to the residues that can be substituted, but the amino acid residues that can be substituted are not limited to those described below:

Group A: leucine, isoleucine, norleucine, valine, norvaline, alanine, 2-aminobutyric acid, methionine, O-methylserine, tert-butylglycine, tert-butylglycine and cyclohexylalanine;

Group B: aspartate, glutamate, isoaspartate, isoglutamate, 2-aminoadipate and 2-aminosuberic acid;

Group C: asparagine and glutamine;

Group D: lysine, arginine, ornithine, 2,4-diaminobutyric acid or 2,3-diaminopropionic acid;

Group E: proline, 3-hydroxyproline and 4-hydroxyproline;

Group F: serine, threonine and homoserine;

Group G: phenylalanine and tyrosine.

In some embodiments of the first aspect, the polypeptide is an isolated polypeptide.

In the second aspect, this application provides a nucleic acid molecule that encodes the polypeptide A described in the first aspect, or encodes the polypeptide A and the polypeptide B described in the first aspect,

The polypeptide B comprises the amino acid sequence shown in SEQ ID NO: 103 or a functional variant thereof, and the functional variant has xanthine amide hydrolase activity.

In some embodiments of the second aspect, the nucleic acid molecule of the present application comprises a nucleotide sequence, this sequence can hybridize under stringent conditions to a nucleotide sequence encoding the polypeptide shown in any one of SEQ ID NO: 1 and SEQ ID NO: 2-102. Or the nucleic acid molecule comprises a nucleotide sequence that specifically hybridizes with a nucleotide sequence that encodes the polypeptide shown in any one of SEQ ID NO: 1 and SEQ ID NO: 2-102, and also this nucleotide sequence encodes a polypeptide that is functionally equivalent to the polypeptide shown in any one of SEQ ID NO: 1 and SEQ ID NO: 2-102.

In some embodiments of the second aspect, the nucleic acid molecule of the present application comprises a nucleic acid that hybridizes under stringent conditions to a nucleotide sequence encoding the polypeptide shown in any one of SEQ ID NO: 1 and SEQ ID NO: 2-102, and simultaneously encodes the polypeptide shown in any one of SEQ ID NO: 103 and SEQ ID 106-206. Or the nucleic acid molecule is comprised of a nucleotide sequence that specifically hybridizes with the nucleotide sequence that encodes the polypeptide shown in any one of SEQ ID NO: 1 and SEQ ID NO: 2-102, and the nucleotide sequence simultaneously encodes the polypeptide shown in any one of SEQ ID NO: 103 and SEQ ID NO: 106-206. And the nucleotide sequence encodes a polypeptide that is functionally equivalent to the polypeptide shown in any one of SEQ ID NO: 1 and SEQ ID NO: 2-102, and simultaneously encodes a polypeptide that is functionally equivalent to the polypeptide shown in any one of SEQ ID NO: 103 and SEQ ID 106-206.

Those skilled in the art can routinely select stringent conditions for DNA hybridization. Generally longer probes require higher temperatures for proper annealing, while shorter probes require lower temperatures. Hybridization generally depends on the ability of denatured DNA to reanneal when the complementary strand is in an environment below its melting temperature. The higher the degree of homology between the probe and the hybridizable sequence, the higher the relative temperature that can be used. Therefore, a higher relative temperature tends to make the reaction conditions more stringent, while at a lower temperature, the stringency is lower. For a detailed description of the stringent conditions of the hybridization reaction, please refer to Ausubel et al., Current Protocols in Molecular Biology, Wiley Interscience Publishers, (1995).

In some embodiments of the second aspect, stringent conditions used for DNA hybridization include: 1) Low ionic strength and high temperature are used for washing, for example, 0.015M sodium chloride/0.0015M sodium citrate/0.1% Sodium alkyl sulfate at 50° C.; 2) Use formamide and other denaturants during hybridization, such as 50% (v/v) formamide plus 0.1% bovine serum albumin/0.1% Ficoll/0.1% polydiene pyrrolidone/pH 6.5 50 mM sodium phosphate buffer and 750 mM sodium chloride, 75 mM sodium citrate; or (3) hybridize overnight at 42° C., the hybridization solution contains 50% formamide, 5×SSC (0.75M sodium chloride, 0.075M citric acid Sodium), 50 mM sodium phosphate (pH 6.8), 0.1% sodium pyrophosphate, 5×Denhardt's solution, sonicated salmon sperm DNA (50 mg/mL), 0.1% SDS and 10% dextran sulfate, then use 0.2×SSC (Sodium chloride/sodium citrate) for washing at 42° C. for 10 minutes, and then washed with high stringency at 55° C. with 0.1×SSC containing EDTA. The moderately stringent conditions can be determined as described in Sambrook et al., Molecular Cloning: A Laboratory Manual, New York: Cold Spring Harbor Press, 1989. Moderately stringent conditions include the use of washing solutions and hybridization conditions (such as temperature, ionic strength, and SDS percentage) that are less stringent than those described above. For example, moderately stringent conditions include hybridization with at least about 16% v/v to 30% v/v formamide and at least about 0.5 M to 0.9 M salt at 42° C., and at least about 0.1 M to 0.2M salt washed at 55° C. Moderately stringent conditions can also include hybridization with 1% bovine serum albumin (BSA), 1 mM EDTA, 0.5M NaHPO₄ (pH 7.2), 7% SDS at 65° C., and (i) 2×SSC, 0.1% SDS; Or (ii) 0.5% BSA, 1 mM EDTA, 40 mM NaIPO₄ (pH 7.2), 5% SDS wash at 60-65° C. Professionals will adjust the temperature and ionic strength according to the length of the probe and other factors. The stringency of hybridizing nucleic acids depends on the length and degree of complementarity of the nucleic acid molecules, as well as other variables well known in the art. The greater the similarity or homology between two nucleotide sequences, the greater the Tm of nucleic acid hybrids containing these sequences. The relative stability of nucleic acid hybridization (corresponding to higher Tm) decreases in the following order: RNA:RNA, DNA:RNA, DNA:DNA. Preferably, the minimum length of a hybridizable nucleic acid is at least about 12 nucleotides, preferably at least about 16, more preferably at least about 24, and most preferably at least about 36 nucleotides.

The nucleic acid molecules of the present application can be combined with other DNA sequences, such as promoters, polyadenylation signals, other restriction sites, multiple cloning sites, other coding segments, etc., so that their total length can vary significantly. It is therefore considered that polynucleotide fragments of almost any length can be utilized; the total length is preferably limited by the ease of preparation and use in the intended recombinant DNA protocol.

Any one of a variety of mature technologies known and available in the art can be used to prepare, manipulate, and/or express polynucleotides and their fusions. For example, nucleic acid molecules encoding the polypeptides of the present application or functional variants thereof can be used in recombinant DNA molecules to direct the expression of the polypeptides in appropriate host cells. Due to the inherent degeneracy of the genetic code, other DNA sequences encoding substantially the same or functionally equivalent amino acid sequences can also be used in this application, and these sequences can be used to clone and express a given polypeptide.

In addition, methods known in the art can be used to modify the nucleic acid molecules of the present application, including but not limited to the cloning, processing, expression and/or activity modification of gene products.

In some embodiments of the second aspect, the nucleic acid molecule is produced by artificial synthesis, such as direct chemical synthesis or enzymatic synthesis.

In some embodiments of the second aspect, the nucleic acid molecule is produced by recombinant technology.

In some embodiments of the second aspect, the nucleic acid molecule is an isolated nucleic acid molecule.

In the third aspect, the present application provides an expression cassette, which contains the nucleic acid molecule described in the second aspect.

In some specific embodiments of the third aspect, the expression cassette may additionally include a 5′ leader sequence, which can play a role in enhancing translation.

When preparing the expression cassette, various DNA fragments can be manipulated to provide the DNA sequence in the proper orientation and in the proper reading frame when appropriate. To achieve this goal, adaptors or linkers can be used to connect DNA fragments, or other operations can be involved to provide convenient restriction sites, remove excess DNA, remove restriction sites, and so on. For this purpose, it may involve in vitro mutagenesis, primer repair, restriction, annealing, and replacement, such as transition and transversion.

In the fourth aspect, the present application provides an expression vector, which comprises the nucleic acid molecule described in the second aspect or the expression cassette described in the third aspect.

In some embodiments of the fourth aspect, any suitable expression vector can be used in this application. For example, the expression vector can be any of pET28, pET14, HT-1N-TAG-2691, pRS416 and other vectors.

In some embodiments of the fourth aspect, the nucleic acid molecule encoding the polypeptide shown in any one of SEQ ID NO: 1-102 is cloned into a vector to form a recombinant vector containing the nucleic acid molecule described in the present application.

In some embodiments of the fourth aspect, it encodes the polypeptide shown in any one of SEQ ID NO: 1-102, and simultaneously encodes the polypeptide shown in any one of SEQ ID NO: 103 and SEQ ID NO: 106-206 The nucleic acid molecule is cloned into a vector to form a recombinant vector containing the nucleic acid molecule described in this application.

In some embodiments of the fourth aspect, the expression vector used to clone the polynucleotide is a plasmid vector.

In some embodiments of the fourth aspect, the above-mentioned expression vector further comprises a control sequence for regulating the expression of the nucleic acid molecule, wherein the nucleic acid molecule is operably linked to the control sequence.

The term “regulatory sequence” as used herein refers to a polynucleotide sequence required to achieve expression of a coding sequence linked to it. The nature of such regulatory sequences varies with the host organism. In prokaryotes, such regulatory sequences generally include promoters, ribosome binding sites and terminators; in eukaryotes, such regulatory sequences generally include promoters, terminator, and in some cases enhancers. Therefore, the term “regulatory sequence” includes all sequences whose existence is the minimum necessary for the expression of the target gene, and may also include other sequences whose existence is advantageous for the expression of the target gene, such as leader sequences.

The term “operably linked” as used herein refers to a situation where the involved sequences are in a relationship that allows them to function in a desired manner. Thus, for example, a regulatory sequence “operably linked” to a coding sequence allows the expression of the coding sequence to be achieved under conditions compatible with the regulatory sequence.

In some embodiments of the fourth aspect, methods well known to those skilled in the art are used to construct a nucleotide sequence that encodes the amino acid sequence shown in any one of SEQ ID NO: 1-102 and an expression vector suitable for transcription/translation regulatory elements. In some embodiments of the fourth aspect, a method well-known to those skilled in the art is used to construct a nucleotide sequence that encodes an amino acid sequence shown in any one of SEQ ID NO: 1-102, and at the same time encodes the amino acid sequence shown in any one of SEQ ID NO: 103 and SEQ ID NO: 106-206 and an expression vector suitable for transcription/translation regulatory elements. These methods include in vitro recombinant DNA technology, DNA synthesis technology, in vivo recombination technology, etc. (Sambroook, et al. Molecular Cloning, a Laboratory Manual, Cold Spring Harbor Laboratory. New York, 1989). The nucleotide sequence is operably linked to an appropriate promoter in the expression vector to direct mRNA synthesis. Representative examples of these promoters include: Escherichia coli lac or trp promoter; lambda phage PL promoter; eukaryotic promoters include CMV immediate early promoter, HSV thymidine kinase promoter, early and late SV40 promoter, retrovirus LTRs and some other known promoters that can control gene expression in prokaryotic cells or eukaryotic cells or their viruses. The expression vector also includes a ribosome binding site for translation initiation and a transcription terminator. Inserting an enhancer sequence into the vector will enhance its transcription in higher eukaryotic cells. Enhancers are cis-acting factors of DNA expression, usually about 10 to 300 base pairs, acting on promoters to enhance gene transcription. Examples include SV40 enhancers of 100 to 270 base pairs on the late side of the replication initiation point, polyoma enhancers and adenovirus enhancers on the late side of the replication initiation point, etc.

In addition, the expression vector preferably contains one or more selectable marker genes to provide phenotypic traits for selection of transformed host cells, such as those encoding kanamycin sulfate resistance, ampicillin resistance, etc.

In the fifth aspect, the present application provides a host cell, which comprises the nucleic acid molecule described in the second aspect or the expression cassette described in the third aspect or the expression vector described in the fourth aspect.

In some embodiments of the fifth aspect, the host cell can express and produce: polypeptide A, which comprises the amino acid sequence shown in SEQ ID NO:1 or a functional variant thereof, wherein the functional variant has 4-urea 5-carboxyimidazole amide hydrolase activity.

In some embodiments of the fifth aspect, the host cell can express and produce: polypeptide A, which comprises the amino acid sequence shown in SEQ ID NO:1 or a functional variant thereof, wherein the functional variant has 4-urea 5-carboxyimidazole amide hydrolase activity; and

Polypeptide B, which comprises the amino acid sequence shown in SEQ ID NO: 103 or a functional variant thereof, wherein the functional variant has xanthine amide hydrolase activity.

In some embodiments of the fifth aspect, the polypeptide A has a catalytic site A defined as follows in the spatial conformation:

The catalytic site A includes catalytic triads close to each other in spatial conformation.

In some embodiments of the fifth aspect, the catalytic triad is composed of C134, D10, and K101 with reference to SEQ ID NO:1.

In some embodiments of the fifth aspect, the catalytic site A further comprises one divalent metal ion.

In some embodiments of the fifth aspect, the catalytic site A further includes one divalent metal ion, such as Zn²⁺ or Mn²⁺.

In some embodiments of the fifth aspect, the catalytic site A further comprises four amino acid residues coordinated with the metal ion, such as H61, H74, E59 and D67.

In some embodiments of the fifth aspect, the usable host cell is a cell containing the above-mentioned expression vector, which may be a eukaryotic cell, for example, a yeast cell culture system may be used for the expression of the polypeptide of the present application. The host cell may also be a prokaryotic cell containing the above-mentioned expression vector, for example, may be selected from the genus Escherichia (for example, Escherichia coli), Lactobacillus, Bifidobacterium, Bacteroides, Firmicutes, etc.

In some specific embodiments of the fifth aspect, the host cell is a yeast cell or E. coli.

In some specific embodiments of the fifth aspect, the nucleic acid molecule encoding one or more enzymes of the present application may exist in the host cell in the form of an episomal vector, or may also be integrated into the genome of the host cell.

In some embodiments of any of the above aspects, the isolated nucleic acid is operably linked to a regulatory sequence, which can be recognized by a host cell transformed with the expression vector.

The expression vector can be introduced into the host cell using any technique known in the art, including transformation, transduction, transfection, viral infection, particle bombardment or Ti-mediated gene transfer. Specific methods include calcium phosphate transfection, DEAE-dextran-mediated transfection, lipofection or electroporation (Davis, L., Dibner, M., Battey, I., Basic Methods in Molecular Biology, (1986)). As an example, when the host is a prokaryotic organism such as Escherichia coli, competent cells can be harvested after the exponential growth phase and transformed by the CaCl₂ method well known in the art.

In the sixth aspect, this application provides a pharmaceutical composition or health food, which comprises the polypeptide A and optional polypeptide B described in the first aspect, the nucleic acid molecule described in the second aspect, the expression cassette described in the third aspect, and the expression vector described in the fourth aspect or the host cell and pharmaceutically acceptable carrier or excipient described in the fifth aspect.

The polypeptide B comprises the amino acid sequence shown in SEQ ID NO: 103 or a functional variant thereof, and the functional variant has xanthine amide hydrolase activity.

In some embodiments of the sixth aspect, the pharmaceutical composition or health food comprises the polypeptide A and the polypeptide B described in the first aspect, and a pharmaceutically acceptable carrier or excipient,

The polypeptide B comprises the amino acid sequence shown in SEQ ID NO: 103 or a functional variant thereof, and the functional variant has xanthine amide hydrolase activity.

In some embodiments of the sixth aspect, the pharmaceutical composition or health food is used for the prevention, intervention and/or treatment of gout.

In some embodiments of the sixth aspect, the pharmaceutical composition or health food may further comprise one or more of the following: lubricants, such as talc, magnesium stearate and mineral oil; wetting agents; emulsifiers; suspending agents; preservatives, such as benzoic acid, sorbic acid and calcium propionate; sweeteners and/or flavoring agents, etc.

In some embodiments of the sixth aspect, the pharmaceutical composition or health food in the present application can be formulated into tablets, pills, powders, lozenges, elixirs, suspensions, emulsions, solutions, syrups, suppositories or capsules, etc.

In some embodiments of the sixth aspect, any method that can be administered to the intestinal tract can be used to deliver the pharmaceutical composition or health food of the present application, preferably oral administration.

In some specific embodiments of the sixth aspect, the host cells of the fifth invention are prepared into enteric-coated capsules for oral administration.

In some embodiments of the sixth aspect, the drug for therapeutic use can be formulated in the form of a lyophilized preparation or an aqueous solution by mixing a reagent with the required purity with a pharmaceutically acceptable carrier, excipient, etc. as appropriate. The composition is used for storage.

In the seventh aspect, this application provides the polypeptide A and optional polypeptide B described in the first aspect, the nucleic acid molecule described in the second aspect, the expression cassette described in the third aspect, the expression vector described in the fourth aspect, and the host cell described in the fifth aspect or the pharmaceutical composition or health food described in the sixth aspect in degrading 4-ureido-5-carboxyimidazole.

The polypeptide B comprises the amino acid sequence shown in SEQ ID NO: 103 or a functional variant thereof, and the functional variant has xanthine amide hydrolase activity.

In some embodiments of the seventh aspect, the application provides the use of the polypeptide A and the polypeptide B described in the first aspect in the degradation of 4-ureido-5-carboxyimidazole.

The polypeptide B comprises the amino acid sequence shown in SEQ ID NO: 103 or a functional variant thereof, and the functional variant has xanthine amide hydrolase activity.

In some embodiments of the seventh aspect, 4-ureido-5-carboxyimidazole is the product of polypeptide B degradation of xanthine.

In some embodiments of the seventh aspect, the degradation of 4-ureido-5-carboxyimidazole occurs in vitro.

In some embodiments of the seventh aspect, the degradation of 4-ureido-5-carboxyimidazole is carried out in an oxygen-free environment, such as in the intestinal tract.

In the eighth aspect, this application provides the polypeptide A and optional polypeptide B described in the first aspect, the nucleic acid molecule described in the second aspect, the expression cassette described in the third aspect, the expression vector described in the fourth aspect, and the host cell described in the fifth aspect or the pharmaceutical composition or health food described in the sixth aspect in the preparation of a medicament for the prevention, intervention and/or treatment of gout.

The polypeptide B comprises the amino acid sequence shown in SEQ ID NO: 103 or a functional variant thereof, and the functional variant has xanthine amide hydrolase activity.

In some embodiments of the eighth aspect, the application provides the use of the polypeptide A and the polypeptide B described in the first aspect in the preparation of a medicament for the prevention, intervention and/or treatment of gout.

The polypeptide B comprises the amino acid sequence shown in SEQ ID NO: 103 or a functional variant thereof, and the functional variant has xanthine amide hydrolase activity.

In the ninth aspect, the present application provides a method to an individual in need for preventing, intervening and/or treating gout, which comprises administering the polypeptide A and optional polypeptide B of the first aspect, the nucleic acid molecule of the second aspect, the expression cassette of the third aspect, the expression vector of the fourth aspect, the host cell of the fifth aspect, or the pharmaceutical composition or health food of the sixth aspect.

The polypeptide B comprises the amino acid sequence shown in SEQ ID NO: 103 or a functional variant thereof, and the functional variant has xanthine amide hydrolase activity.

In some embodiments of the ninth aspect, the present application provides a method for preventing, intervening and/or treating gout to an individual in need, which comprises administering the polypeptide A and the polypeptide B of the first aspect.

The polypeptide B comprises the amino acid sequence shown in SEQ ID NO: 103 or a functional variant thereof, and the functional variant has xanthine amide hydrolase activity.

In some embodiments of any of the above aspects, wherein the polypeptide B has a catalytic site B defined as follows in a spatial conformation:

The catalytic site B comprises amino acid residues close to each other in spatial conformation, referring to the amino acid residues of H59, H61, K151, H186, H242 and D316 of SEQ ID NO:103.

In some embodiments of any of the above aspects, the catalytic site B further comprises one divalent metal ion.

In some embodiments of any of the above aspects, the catalytic site B further includes two divalent metal ions (for example, Zn²⁺ and/or Mn²⁺).

In some embodiments of any of the above-mentioned aspects, wherein the polypeptide B further comprises a binding site B having the following definition in a spatial conformation:

The binding site B includes amino acid residues I288, A289, P338 and G339 of SEQ ID NO: 103 that are close to each other in spatial conformation.

In some embodiments of any of the above aspects, wherein the distance between the catalytic site B and the binding site B is not more than 5 angstroms.

In some embodiments of any of the above aspects, wherein the functional variant having xanthine amide hydrolase activity is a natural isoenzyme of the amino acid sequence shown in SEQ ID NO: 103.

In some embodiments of any of the above aspects, the natural isoenzyme of the amino acid sequence shown in SEQ ID NO: 103 comes from: Bacillus firmus, compost metagenome, Clostridium purinilyticum, Thermoflavimicrobium sp., Marininema halotolerans, Clostridiaceae bacterium, Bacillus sp., Paenibacillus sp., Thermoflavimicrobium dichotomicum, Fictibacillus enclensis, Marininema mesophilum, Paenibacillus typhae, Tissierella praeacuta, Bradyrhizobium japonicum, Acidaminobacter hydrogenoformans, Caloranaerobacter sp., Tissierella sp., Paenibacillus donghaensis, Gottschalkia acidurici, Clostridium acidurici, Bacillus fortis, Bacillus oceanisediminis, Virgibacillus profundi, Anaeromicrobium sediminis, Thermosyntropha lipolytica, Alkaliphilus peptidifermentans, Aneurinibacillus migulanus, Bacillus bacterium, Marinisporobacter balticus, Bacillus terrae, Tindallia calforniensis, Romboutsia lituseburensis, Paraclostridium bifermentans, Bacillus praedii, Carbydothermus islandicus, Caloramator australicus, Paraclostridium bifermentans, Tepidimicrobium xylanilyticum, Bacillus notoginsengisoli, [Clostridium] ultunense Esp, Bacillus freudenreichii, Caloramator fervidus, Soehngenia saccharolytica, Thermotalea metallivorans, Ornithinibacillus halophilus, Fictibacillus sp., Bacillus mesonae, Tindallia magadiensis, Paenibacillus borealis, Paraclostridium benzoelyticum, Bacillus solani, Thermohalobacter berrensis, Acinetobacter sp., Maledivibacter halophilus, Tissierella creatinine, Natronincola peptidivorans, Anaerovirgula multivorans, Carbydothermus hydrogenoformans, Sporanaerobacter acetigenes, Proteiniborus sp., Virgibacillus indicus, Andreesenia angusta, Paludifilum, halophilum, Proteiniborus ethanoligenes, Alkaliphilus metalliredigens, Fictibacillus solisalsi, Sporosarcina globispora, Alkaliphilus sp., Alkaliphilus oremlandii, Caloramator mitchellensis, Clostridium cylindrosporum, Ammoniphilus sp., Carbydothermus pertinax, Soehngenia sp. and Natribacillus halophilus.

In some specific embodiments of any of the above aspects, the natural isoenzyme of the amino acid sequence shown in SEQ ID NO: 103 includes the amino acid sequence shown in any one of SEQ ID NO: 106-206.

In some embodiments of any of the above aspects, wherein the functional variant with xanthine amide hydrolase activity has insertion, substitution and/or deletion of one or more amino acids of the amino acid sequences shown in SEQ ID NO: 103 or its natural isoenzymes.

In some embodiments of any of the above aspects, the insertion, substitution and/or deletion does not occur at the catalytic site B.

In some embodiments of any of the above aspects, the insertion, substitution and/or deletion does not occur at binding site B.

In some embodiments of any of the above aspects, the insertion, substitution and/or deletion does not occur at the catalytic site B and the binding site B.

In some specific embodiments, the host cell can express and produce: polypeptide A, which comprises the amino acid sequence shown in SEQ ID NO:1 or a functional variant thereof, wherein the functional variant has 4-ureido-5-carboxyimidazole amide hydrolase activity, and the polypeptide A has a catalytic site A and a binding site A defined as follows in the spatial conformation:

The catalytic site A comprises the catalytic triads C134, D10, K101 and one divalent metal ion (e.g. Zn²⁺ or Mn²⁺) that are close to each other in spatial conformation, with reference to SEQ ID NO:1, and the H61, H74, E59, D67 amino acid residues coordinated by divalent metal ions; and

The binding site A comprises amino acid residues F15, R71, V129, H104 and W130 that are close to each other in spatial conformation refer to SEQ ID NO:1.

In some specific embodiments, the host cell is capable of expressing and producing:

Polypeptide A, which comprises the amino acid sequence shown in SEQ ID NO:1 or a functional variant thereof, wherein the functional variant has 4-ureido-5-carboxyimidazole amide hydrolase activity and has the following definition of the catalytic site A and binding site A in spatial conformation:

The catalytic site A comprises the catalytic triads C134, D10, K101 and one divalent metal ion (e.g. Zn²⁺ or Mn²⁺) that are close to each other in spatial conformation, with reference to SEQ ID NO:1, and the H61, H74, E59, D67 amino acid residues coordinated by divalent metal ions; and

The binding site A comprises amino acid residues F15, R71, V129, H104 and W130 that are close to each other in spatial conformation refer to SEQ ID NO:1;

as well as

Polypeptide B, which comprises the amino acid sequence shown in SEQ ID NO: 103 or a functional variant thereof, wherein the functional variant has xanthine amide hydrolase activity, and has a catalytic site B and a binding site B defined as follows in the spatial conformation:

The catalytic site B comprises amino acid residues H59, H61, K151, H186, H242, and D316 that are close to each other in spatial conformation with reference to SEQ ID NO: 103 and two divalent metal ions (e.g., Zn²⁺ and/or Mn²⁺); and

The binding site B includes amino acid residues I288, A289, P338 and G339 of SEQ ID NO: 103 that are close to each other in spatial conformation.

As an exemplary embodiment, in the anaerobic degradation of xanthine, the xanthine amide hydrolase shown in SEQ ID NO: 103 degrades xanthine into 4-ureido-5-carboxyimidazole, the 4-ureido-5-carboxyimidazole amide hydrolase shown in SEQ ID NO: 1 degrades 4-ureido-5-carboxyimidazole into 4-amino-5-carboxyimidazole, thereby reducing human cells' acquisition of purines and converting them into uric acid. The pathway degrades xanthines in the intestine to prevent, intervene and/or treat gout.

### EXAMPLE

The following examples are only illustrative, and are not intended to limit the scope of the embodiments of the present application or the scope of the appended claims.

### Example 1. Preparation of Gene Clone Encoding Xanthine Amide Hydrolase

Using the genome of Bacillus firmus (CGMCC 1.2010) as a template, using the upstream primers and downstream primers shown in SEQ ID NO: 104 and 105 to PCR amplify the xanthine amide hydrolase (SEQ ID NO: 103) nucleic acid molecules to obtain the gene sequence encoding xanthine amide hydrolase. Using Gibson's method, the gene sequence encoding xanthine amide hydrolase was inserted into the SspI restriction site of the HT-1N-TAG-2691 vector (which contains His₆ tag and TEV protease cleavage site) to obtain an expression vector containing a clone encoding xanthine amide hydrolase.

### Example 2. Recombinant Expression and Purification of Xanthine Amide Hydrolase

2.1 Recombinant Expression

The expression vector containing gene 1 encoding xanthine amide hydrolase constructed in Example 1 was transformed into Escherichia coli BL21 cells, and spread on LB agar plates containing 50 μg/mL kanamycin sulfate, incubate overnight at 37° C. Pick a single colony and culture it overnight in 5 mL LB liquid medium containing kanamycin sulfate at 37° C. and 220 rpm, and transfer it to 800 mL LB medium for expansion the next day. When the OD 600 value reaches about 0.8, change the temperature to 18° C., and IPTG was added at a final concentration of 0.3 mM to induce protein expression. After 16 hours of induction of expression, the cells were collected by centrifugation at 4000×g for 10 min at 4° C. Resuspend the bacterial precipitate in 35 mL lysis buffer [50 mM Tris/HCl, pH 8.0, 1 mM phenylmethylsulfonyl fluoride (PMSF), 0.2 mg/mL lysozyme, 0.03% Triton X-100 and 1μL DNase I], and frozen and preserved at −80° C.

2.2 Purification

The cells were taken out from −80° C. and incubated in a water bath at 25° C. for 40 minutes to lyse the cells. Add 6 mL of 6% streptomycin sulfate aqueous solution, mix gently, and centrifuge at 20,000×g for 5 minutes at 4° C. The supernatant was filtered with a 0.22 m filter membrane and bound to a 5 mL TALON cobalt column equilibrated with buffer A (20 mM Tris-HCl 7.5, 0.2M KCl), and the column was washed with 10 times the column volume of buffer A. Then, the target protein was eluted with 5 column volumes of buffer B containing 150 mM imidazole (20 mM Tris-HCl 7.5, 0.2 M KCl, 150 mM imidazole, 5 mM 0-mercaptoethanol). Add ammonium sulfate to the eluted protein solution to 70% saturated precipitated protein, centrifuge at 10,000×g for 10 minutes, discard the supernatant, and use 5 mL buffer C (20 mM Tris-HCl 7.5, 0.2M KCl, 10% (V/v) glycerol) resuspending the precipitate, and a G25 column was used to remove the salt. After removing the salt, the target protein is divided into aliquots, quick-frozen with liquid nitrogen, and stored at −80° C. for freezer storage. The final concentration of xanthine amide hydrolase is detected by a spectrophotometer. According to the protein sequence, the extinction coefficient of xanthine amide hydrolase is 55,810M⁻¹ cm⁻¹. On average, about 35 mg of protein can be purified per liter. FIG. 5 shows the process of expression and purification of xanthine amide hydrolase, and its migration rate on gel electrophoresis conforms to the actual molecular weight (55.7 kDa).

### Example 3. Detection of Xanthine Amide Hydrolase Activity

Incubate 1 mL of a solution containing 1 mM xanthine, 4 μM xanthine amide hydrolase obtained in Example 2, 0.1 mM Mn², 200 mM Tris-HCl 7.5 in a water bath at 30° C. for 0.5 h to allow the system to fully react, and then add the equal volume of methanol solution, mixed and centrifuged at 14,000×g for 10 minutes, and the protein precipitate was filtered through a 0.22 m filter membrane, which was recorded as the experimental group. The control group was a reaction without addition of xanthine amide hydrolase and divalent metal ions, and the control group had the same other experimental conditions as the experimental group (Table 1). The experiment was carried out in an anaerobic glove box. All solutions including protein solution, reaction buffer, etc. were deoxygenated through Schlenk line before entering the glove box.

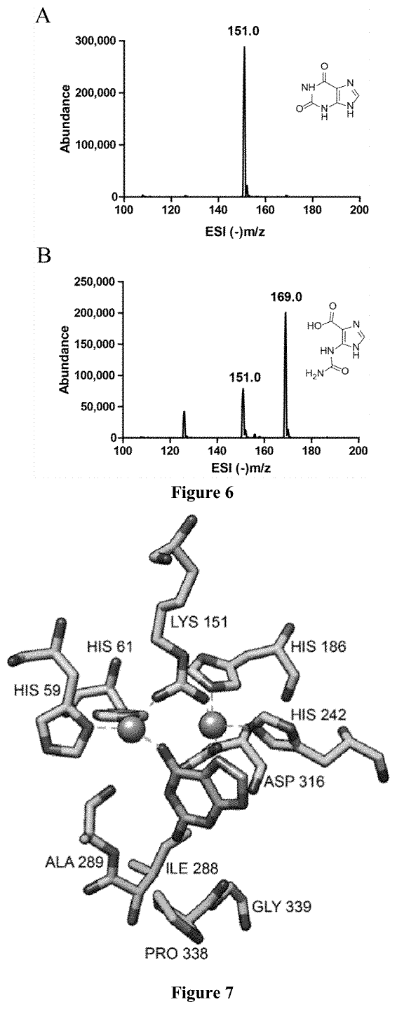

Using LC-MS to detect xanthine amide hydrolase catalyzed the ring opening of xanthine hydrolysis, no product peak was detected in the control group, while the product peak with a molecular weight of 169 Da was detected in the experimental group, which proved that xanthine amide hydrolase can degrade xanthine (FIG. 6).

Table 1

### Example 4. Identification of the Active Center of Xanthine Amide Hydrolase

After confirming in vitro that the polypeptide containing the amino acid sequence shown in SEQ ID NO: 103 has xanthine amide hydrolase activity, the PHYRE2 server was used to simulate the structure of the enzyme (FIG. 7), which showed that it contained four histidines and one histidine. A catalytic site composed of a lysine, an aspartic acid, and two divalent metal ions (Zn²⁺ and/or Mn²⁺). Furthermore, computer software was used to show the anchoring of the substrate xanthine to the simulated xanthine amide hydrolase. The active center also includes an isoleucine, an alanine, a proline and a glycine in the spatial conformation of each other. The binding site close to the composition interacts with the substrate xanthine (FIG. 7).

### Example 5. Isoenzyme Identification of Xanthine Amide Hydrolase

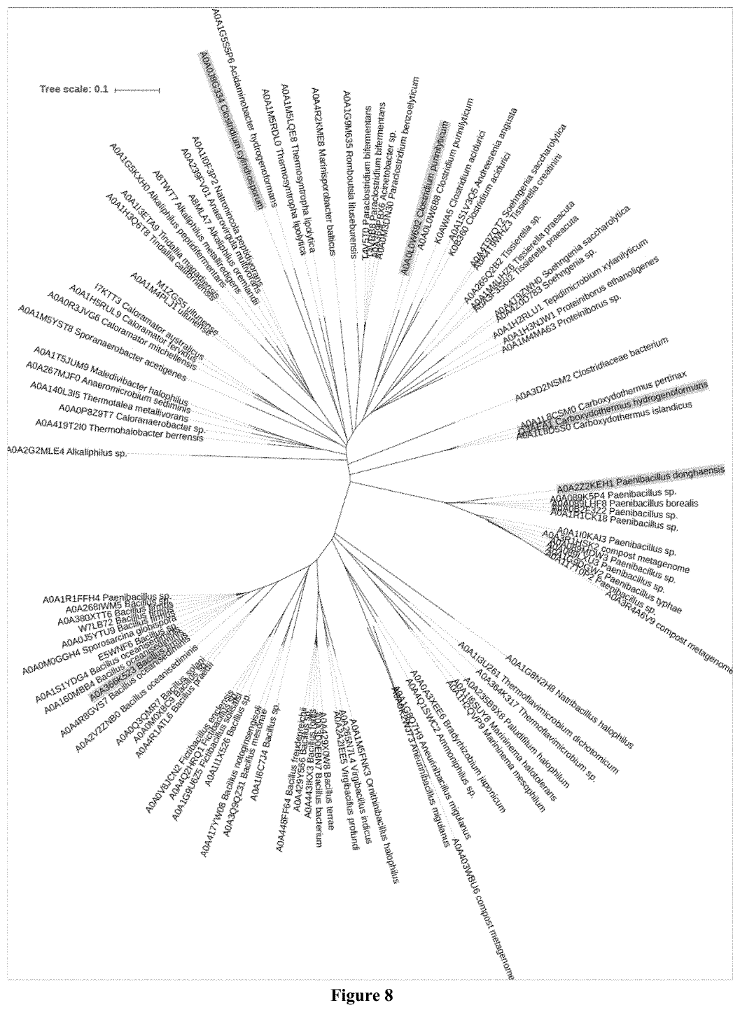

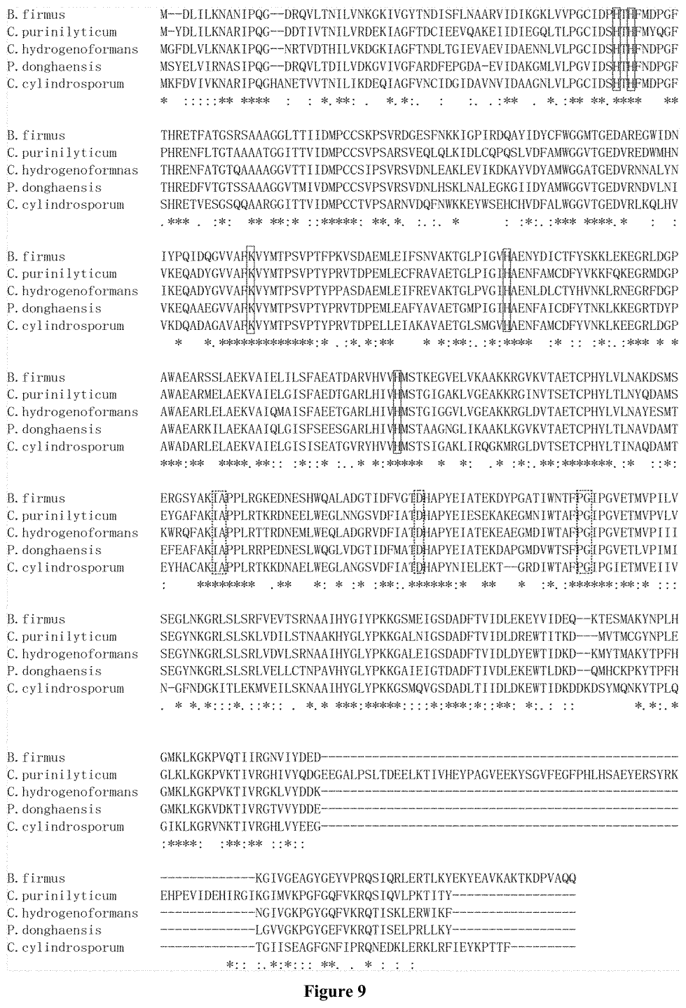

The inventor of the present application further found the sequence of this xanthine amide hydrolase isoenzyme UniRef50 from the Uniprot database (covering 50% sequence identity, and at the same time more than 80% of the xanthine amide hydrolase length of the protein sequence), the above sequences are all in The UniRef50_Q3AEA1 cluster includes a total of 101 protein sequences. The phylogenetic tree analysis of these 101 proteins from different species was carried out (FIG. 8). A representative protein is selected from each evolutionary branch, and there are five proteins in total, which are derived from Bacillus firmus, Clostridium cylindrosporum DSM 605, Clostridium purinilyticum, Carbydothermus hydrogenoformans, strain ATCC BAA-161/DSM 6008/Z-2901 and Paenibacillus donghaensis, the accession numbers encoded by them are A0A366K523, A0A0J8G334, A0A0L0W692, Q3AEA1, and A0A2Z2KEH1. The sequence comparison results show that the amino acid residues at the above catalytic site and binding site are highly conserved, and all five sequences contain the above amino acid residues, proving that these enzymes are xanthine amide hydrolases (FIG. 9). Table 2 shows the accession numbers, sources and amino acid sequence numbers of 101 xanthine amide hydrolase enzymes found in the Uniprot database.

Table 2

### Example 6. Cloning, Expression, Purification and Activity Detection of 4-Ureido-5-Carboxyimidazole Amide Hydrolase

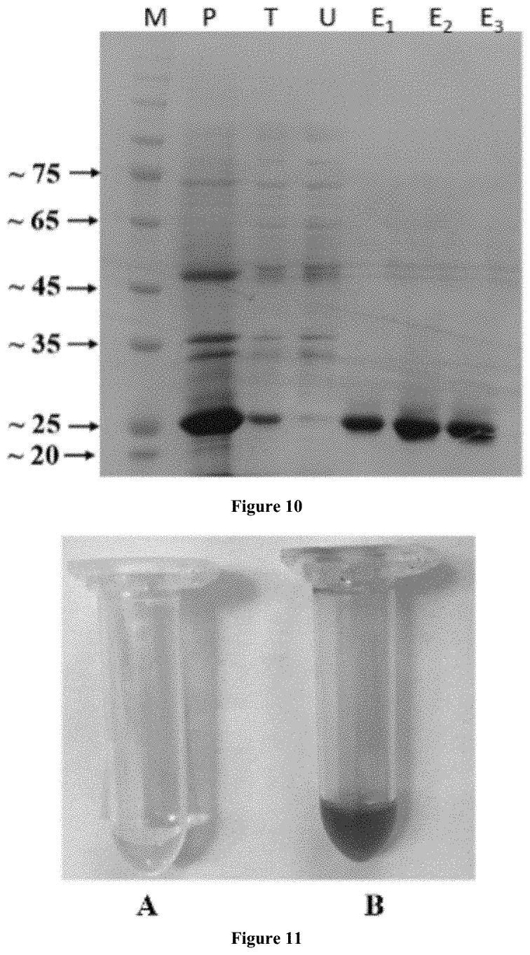

The DNA fragment encoding the 4-ureido-5-carboxyimidazole amide hydrolase of SEQ ID NO:1 was synthesized by Anhui General Company and inserted into the SspI site of the HT-1 vector by the Gibson method, and the vector was transformed into E. coli BL21(DE3)), the induced expression of gene 2 encoding 4-ureido-5-carboxyimidazole amide hydrolase is the same as the induced expression of gene 1 encoding xanthine amide hydrolase. The purification of 4-ureido-5-carboxyimidazole amide hydrolase also uses TALON cobalt affinity column, and the concentration of 4-ureido-5-carboxyimidazole amide hydrolase obtained is 371 μM. The purified 4-ureido-5-carboxyimidazole amide hydrolase (FIG. 10) undergoes a coupling reaction with xanthine amide hydrolase under anaerobic conditions to produce 4-amino-5-carboxyimidazole (the reaction in FIG. 4). 2). Incubate 200 μL of a solution containing 1 mM xanthine, 4 μM xanthine amide hydrolase obtained in Example 2, 0.1 mM Mn²⁺, and 20 mM Tris-HCl 7.5 in a water bath at 30° C. for 0.5 h to fully react. Add 92 μL of H₂O and 8 μL of 4-ureido-5-carboxyimidazole amide hydrolase (final concentration ˜10 μM) to the system, continue to react in a 30° C. water bath for 0.5 h, then add an equal volume of methanol solution to mix, and after centrifugation at 14,000×g for 10 minutes, the protein precipitate was filtered through a 0.22 μm filter membrane, and it was recorded as the experimental group. The control group was a reaction without addition of 4-ureido-5-carboxyimidazole amide hydrolase and divalent metal ions, and the control group had the same other experimental conditions as the experimental group (see Table 3). The experimental group produced the reaction product 4-amino-5-carboxyimidazole, which spontaneously multimerized under aerobic conditions and showed a light purple color, while the control group did not show color (FIG. 11). Such multimerization properties of aminoimidazole compounds are known, see literature: Structural and functional characteristics of various forms of red pigment of yeast Saccharomyces cerevisiae and its synthetic analog. Cell &amp; Tissue Biology 7(1), 86-94.

table 3

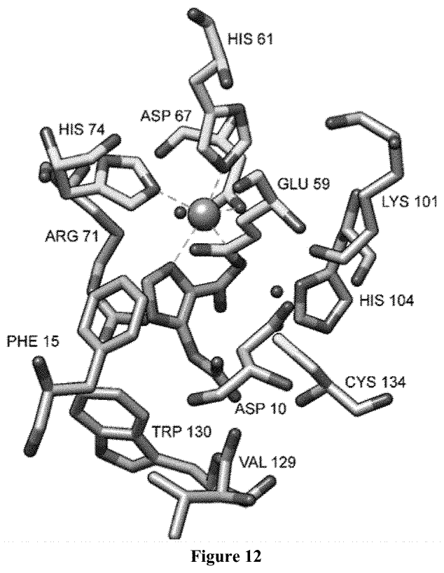

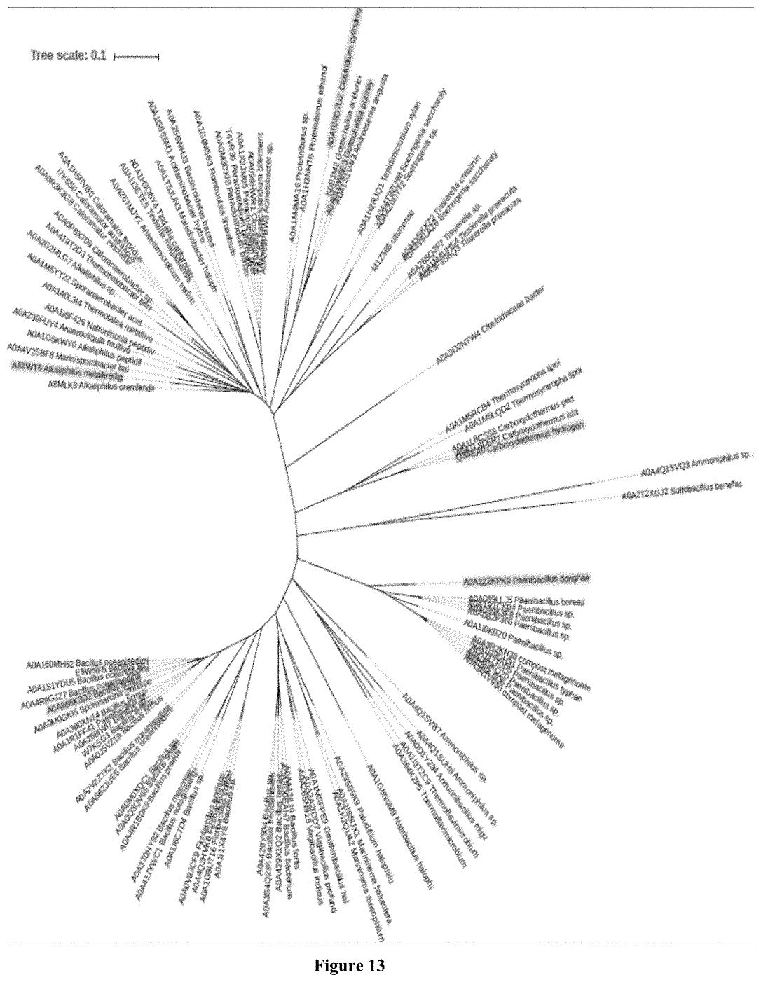

After confirming in vitro that the polypeptide of the amino acid sequence shown in SEQ ID NO:1 has 4-ureido-5-carboxyimidazole amide hydrolase activity, the PHYRE2 server is used to simulate the structure of the enzyme, and the computer software is further used to structure the enzyme simulate and anchor the substrate, and the results show that the active center contains a cysteine (nucleophilic attack), an aspartic acid, a lysine, and a proteolytic enzyme-like catalytic triad, and a bivalent metal ion (Such as Zn²⁺ or Mn²⁺), two histidines, one glutamic acid and one aspartic acid constitute a catalytic site, and an arginine containing 4-ureido-5-carboxyimidazole interaction, a phenylalanine, a histidine, a tryptophan and a valine constitute the binding site (FIG. 12).

### Example 7. Activity Detection of Xanthine Amide Hydrolase and 4-Ureido-5-Carboxyimidazole Amide Hydrolase

7.1 Activity Detection of Xanthine Amide Hydrolase

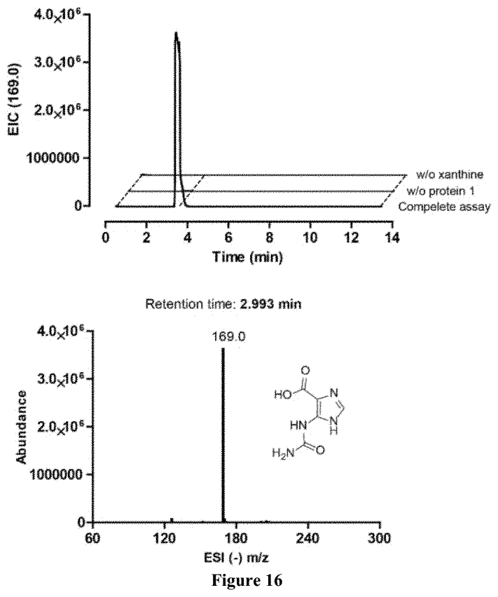

The reaction system of the experimental group contained 10 mM xanthine, 1 mM Mn²⁺, 20 mM Tris-HCl 7.5, 100 mM KCl, and 10 μM xanthine amide hydrolase. The experimental group was reacted in a 30° C. water bath for 30 minutes, and the protein was removed with a 3 kDa concentrated tube. Control groups 1 and 2 were without addition of xanthine amide hydrolase and without addition of substrate xanthine, and the rest of the reaction conditions were the same as those in the experimental group. The product was separated by LC-MS and the molecular weight of the product was detected. Using a C18 chromatographic column, mobile phase A was water, mobile phase B was acetonitrile, 5%-10% acetonitrile eluted for 12 minutes, and 10%-95% acetonitrile eluted 1 minute. Extracted ion chromatogram (EIC) results show that: only the experimental group has the product 4-ureido-5-carboxyimidazole peak with a retention time of 2.993 min, and control groups 1 and 2 have no product 4-ureido-5-carboxyimidazole peak. (See A in FIG. 16). The ESI chart of the mass spectrum of the product 4-ureido-5-carboxyimidazole at a retention time of 2.993 minutes in the negative ion mode of the experimental group showed that its molecular weight was 169 Da (B in FIG. 16).

7.2. Activity Detection of 4-Ureido-5-Carboxyimidazole Amide Hydrolase

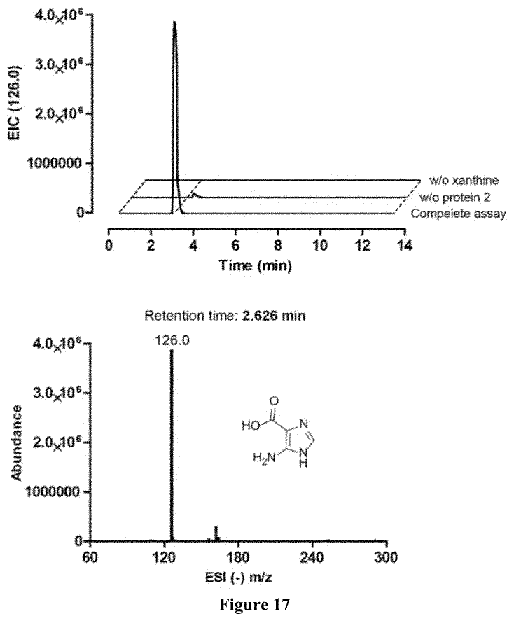

The reaction system of the experimental group contained 10 mM xanthine, 1 mM Mn²⁺, 20 mM Tris-HCl 7.5, 100 mM KCl, 10 μM xanthine amide hydrolase, and 8 μM 4-ureido-5-carboxyimidazole amide hydrolase. The experimental group was reacted in a water bath at 30° C. for 30 minutes, and the protein was removed with a 3 kDa concentrated tube. The control groups 1 and 2 did not add 4-ureido-5-carboxyimidazole amide hydrolase or did not add the substrate xanthine, and the other reaction conditions were the same as the experimental group. The product was separated by LC-MS and the molecular weight of the product was detected. Using a C18 chromatographic column, mobile phase A was water, mobile phase B was acetonitrile, 5%-10% acetonitrile eluted for 12 minutes, and 10%-95% acetonitrile eluted 1 minute. Extracted ion chromatogram (EIC) results show that: only the experimental group has the product 4-amino-5-carboxyimidazole peak with a retention time of 2.626 min, and control groups 1 and 2 have no product 4-amino-5-carboxyimidazole peak (see FIG. 17A). The ESI chart of the mass spectrum of the product 4-amino-5-carboxyimidazole at the retention time of 2.626 min in the negative ion mode of the experimental group showed that its molecular weight was 126 Da (B in FIG. 17).

### Example 8. Isoenzyme Identification of 4-Ureido-5-Carboxyimidazole Amide Hydrolase

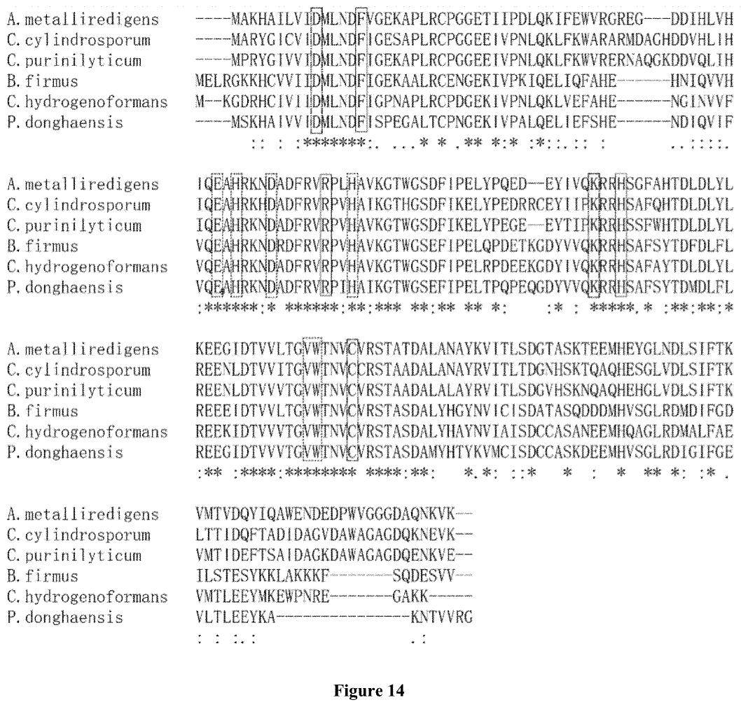

The inventor of the present application further found the sequence of the 4-ureido-5-carboxyimidazole amide hydrolase UniRef50 from the Uniprot database (covering 50% sequence identity, while exceeding 80% length of the 4-ureido-5-carboxyimidazole amide hydrolase protein sequence), the above sequence is classified into five clusters: UniRef50_A6TWT6, UniRef50 A0A1H2QU42, UniRef50 Q3AEA0, UniRef50_A0A3R2KN38 and UniRef50_A0A0R3K3G8, including 102 protein sequences in total (13). A representative protein is selected from each evolutionary branch, which are derived from Alkaliphilus metalliredigens, Bacillus firmus, Paenibacillus donghaensis, Carbydothermus hydrogenoformans (strain ATCC BAA-161/DSM 6008/Z-2901), Gottschalkia purinilytica, and Clostridium cylindrosporum, the 4-ureido-5-carboxyimidazole amide hydrolase accession numbers encoded by them are A6TWT6, A0A366K3D2, A0A2Z2KPK9, Q3AEA0, A0A0L0W6E8 and A0A0L0W6E8—The amino acid sequence of ureido-5-carboxyimidazole amide hydrolase is aligned. The sequence comparison results show that the amino acid residues of the above catalytic site and the binding site are highly conserved, and all six sequences contain the above amino acid residues, proving that these enzymes are 4-ureido-5-carboxyimidazole amide hydrolase (FIG. 14). Table 4 shows the accession numbers, sources, and amino acid sequence numbers of 101 4-ureido-5-carboxyimidazole amide hydrolase isoenzymes found in the Uniprot database.

Table 4

### Example 9. Construction of Escherichia coli Expressing Different Enzyme Combinations

In this example, an Escherichia co/i capable of simultaneously expressing xanthine amide hydrolase (SEQ ID NO: 103) and 4-ureido-5-carboxyimidazole amide hydrolase (SEQ ID NO: 1) was constructed.

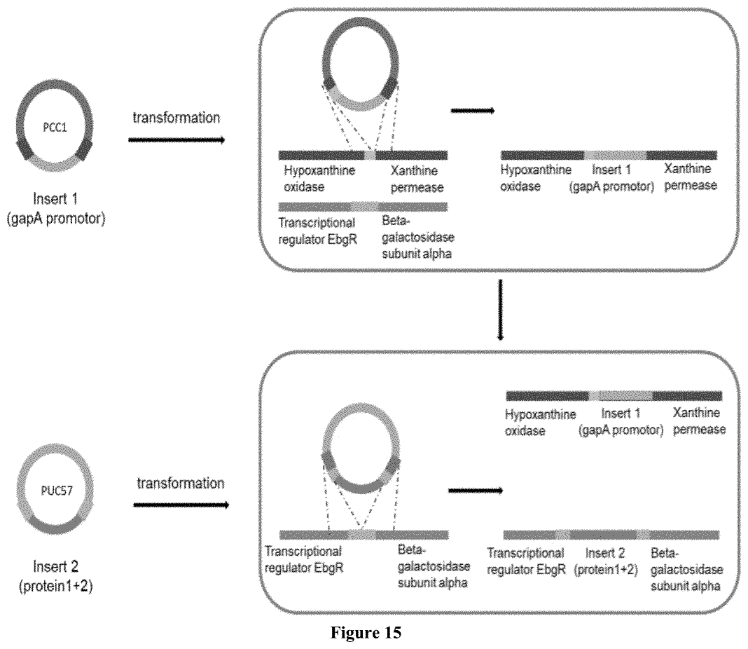

The key operation of this example is to tandem the genes encoding xanthine amide hydrolase and 4-ureido-5-carboxyimidazole amide hydrolase (gene 1 and gene 2) into the E. co/i genome, such as transcriptional regulator EbgR and Beta-galactosidase subunit alpha gene, let it be stably expressed, and at the same time replace the promoter that controls the guanine transporter and guanine deaminase with the gapA promoter, so that the two genes are continuously and stably expressed (FIG. 15).

In this embodiment, it is preferable to replace the promoters encoding guanine transporter and guanine deaminase with the constitutive and continuous and stable expression promoter gapA. The original promoter usually lacks nitrogen sources in the environment and needs to use guanine nitrogen. When the nitrogen source is sufficient, the gene controlled by it is not expressed. In order to make the guanine in the food degraded by the xanthine amide hydrolase of the present invention, it is necessary to replace the control coded guanine transporter and guanine deamination. The promoter of the enzyme gene allows it to be expressed at any time, thereby effectively transporting guanine into the engineered E. co/i cells, and passing guanine deaminase, xanthine amide hydrolase, 4- Urea-5-carboxyimidazole amide hydrolase acts sequentially to degrade, thereby effectively preventing the absorption of guanine in the human intestine and converting it into uric acid.

### Example 10. Escherichia coli Expressing Xanthine Amide Hydrolase and 4-Ureido-5-Carboxyimidazole Amide Hydrolase Simultaneously for the Treatment of Gout

High uric acid rats or uricase knockout transgenic mice induced by oxonic acid were used as gout animal models, and various Escherichia coli prepared in Example 9 were made into enteric-coated capsules. Under the same feeding conditions, test whether various E. coli enteric-coated capsules can reduce the blood uric acid content of gout rats and mouse models.

### Example 11. Escherichia coli Expressing Xanthine Amide Hydrolase and 4-Ureido-5-Carboxyimidazole Amide Hydrolase Simultaneously for the Treatment of Gout

11.1 Rat Experiment

Select SD male rats (body weight 120 g-150 g), 6 rats in each group, under the same feeding conditions, they are divided into experimental group, hyperuricemia group and control group. The specific experimental process is as follows:

1. Animal pre-adaptation: ≥7 days of pre-adaptation process, feeding with ordinary feed and ordinary drinking water.

2. Antibiotic pretreatment: feed with ordinary feed, add 2 mg/mL streptomycin+1 mg/mL ampicillin to the drinking water after feeding rats for 3 days, stop drinking water for more than 6 hours, and then give the subsequent gavage treatment.

3. Experimental group: rats were fed every day with a feed containing 1% (w/w) adenine, and 200 μL containing 2×10\hat{ }10 E. coli suspension prepared in Example 9 was used for gavage treatment for two weeks.

Hyperuricemia group: The rats were fed a diet containing 1% (w/w) adenine every day for two weeks.

Control group: rats were fed with ordinary diet daily for two weeks.

After two weeks of feeding, blood was taken from the eye socket, and the serum uric acid content was detected with a uric acid kit.

11.2 Mouse Experiment

Choose Balb/c mice (8 weeks old), 5 mice in each group, and divide them into experimental group, hyperuricemia group and control group under the same feeding conditions.

The specific experimental process is as follows:

1. Animal pre-adaptation: ≥7 days of pre-adaptation process, feeding with ordinary feed and ordinary drinking water.

2. Antibiotic pretreatment: feed with ordinary feed, add 2 mg/mL streptomycin+1 mg/mL ampicillin to the drinking water. After feeding the mice for 3 days, stop drinking water for more than 6 hours, then give the follow-up gavage treatment.

3. Experimental group: mice were fed every day with 0.1% (w/w) adenine feed and 200 mg/kg potassium oxycyanate by gavage, and 100 μL containing 1×10\hat{ }10 E. coli suspension prepared in Example 9 was used for gavage treatment for two weeks.

Hyperuricemia group: mice were fed 0.1% (w/w) adenine feed and 200 mg/kg potassium oxycyanate by gavage every day for two weeks.

Control group: normal feed was fed every day for two weeks.

After two weeks of feeding, blood was taken from the eye socket, and the serum uric acid content was detected with a uric acid kit.

The exemplary embodiments of the various inventions of the present application are described above. However, without departing from the essence and scope of the present application, those skilled in the art can modify or improve the exemplary embodiments described in the present application. The resulting variants or equivalent solutions also belong to the scope of this application.

1. Polypeptide A, which comprises the amino acid sequence shown in SEQ ID NO:1 or a functional variant thereof, wherein the functional variant has 4-ureido-5-carboxyimidazole amide hydrolase activity.

2. The polypeptide A of claim 1, which has a catalytic site A defined as follows in its spatial conformation: the catalytic site A includes catalytic triads that are close to each other in spatial conformation; optionally, the catalytic triad is composed of C134, D10, and K101 with reference to SEQ ID NO:1.

3. The polypeptide A of claim 2, wherein the catalytic site A further comprises a divalent metal ion, optionally one divalent metal ion (such as Zn²⁺ or Mn²⁺).

4. The polypeptide A of claim 3, wherein the catalytic site A further comprises four amino acid residues coordinated with the metal ion, such as H61, H74, E59 and D67.

5. The polypeptide A according to claim 1, which further comprises a binding site A defined as follows in spatial conformation: the binding site A comprises amino acid residues F15, R71, V129, H104 and W130 that are close to each other in spatial conformation and refer to SEQ ID NO:1.

6. The polypeptide A of claim 5, wherein the distance between the catalytic site A and the binding site A is not more than 5 angstroms.

7. The polypeptide A of claim 1, wherein the functional variant is a natural isoenzyme of the amino acid sequence shown in SEQ ID NO:1; preferably, the natural isoenzymes are from: Clostridium cylindrosporum, Sporanaerobacter acetigenes, Thermosyntropha lipolytica, Aneurinibacillus migulanus, Alkaliphilus oremlandii, Fictibacillus enclensis, Bacillus sp., Bacillus mesonae, Bacillus oceanisediminis, Bacillus firmus, Marinisporobacter balticus, Alkaliphilus peptidifermentans, Thermoflavimicrobium dichotomicum, Paenibacillus sp., Caloranaerobacter sp., Virgibacillus profundi, Carboxydothermus islandicus, Natronincola peptidivorans, Paenibacillus typhae, Natribacillus halophilus, Caloramator australicus, Carbydothermus hydrogenoformans, Paraclostridium benzoelyticum, Gottschalkia acidurici, Bacillus notoginsengisoli, Romboutsia lituseburensis, Carboxydothermus pertinax, Acidaminobacter hydrogenoformans, Soehngenia saccharolytica, Tindallia californiensis, Sulfobacillus benefaciens, Bacillus solani, Clostridium sp., Caloramator mitchellensis, Sporosarcina globispora, Paraclostridium bifermentans, Proteiniborus sp., Maledivibacter halophilus, Tepidimicrobium xylanilyticum, Tissierella sp., Andreesenia angusta, Ammoniphilus sp., Marininema halotolerans, Marininema mesophilum, Clostridiaceae bacterium, compost metagenome, Thermotalea metallivorans, Caloramator fervidus, Paludifilum halophilum, Bacillus fortis, Fictibacillus sp., Bacillus terrae, Tissierella creatinini, Virgibacillus indicus, Bacillus praedii, Fictibacillus solisalsi, Acinetobacter sp., Anaerovirgula multivorans, Alkaliphilus sp., Paenibacillus donghaensis, Paenibacillus borealis, Thermohalobacter berrensis, Tindallia magadiensis, Ornithinibacillus halophilus, Soehngenia sp., Proteiniborus ethanoligenes, Anaeromicrobium sediminis, Bacillus freudenreichii, Alkaliphilus metalliredigens, Thermoflavimicrobium sp., Bacteroidetes bacterium, Gottschalkia purinilytica, Bacillus bacterium and Paraclostridium bifermentans; more preferably, the natural isoenzyme comprises the amino acid sequence shown in any one of SEQ ID NO: 2-102.

8. The polypeptide A according to claim 1, wherein the functional variant is shown in SEQ ID NO:1, one or more amino acids insertion, substitution, and/or deletion occurs based on the amino acid sequence or its natural isoenzyme, optionally, the insertion, substitution, and/or deletion does not occur in the catalytic site A and/or the binding site A.

9. A nucleic acid molecule that encodes the polypeptide A according to claim 1.

10. An expression cassette comprising the nucleic acid molecule of claim 9.

11. An expression vector comprising the nucleic acid molecule of claim 9.

12. A host cell comprising the nucleic acid molecule of claim 9.

13. The host cell of claim 12, which is a eukaryotic cell or a prokaryotic cell; preferably, the eukaryotic cell is a yeast cell; preferably, the prokaryotic cell is selected from Escherichia, Lactobacillus, Bifidobacterium, Bacteroides and Firmicutes; more preferably, the Escherichia is Escherichia coli.

14. A pharmaceutical composition or health food, which comprises the polypeptide A and optional polypeptide B according to claim 1.

15. The pharmaceutical composition or health food according to claim 14, which is used for the prevention, intervention and/or treatment of gout.

16. The polypeptide A and optional polypeptide B according to claim 1, wherein the polypeptide B comprises the amino acid sequence shown in SEQ ID NO: 103 or a functional variant thereof, and the functional variant has xanthine amide hydrolase activity.

17. The usage according to claim 16, wherein the degradation of 4-ureido-5-carboxyimidazole occurs in vitro.

18. The polypeptide A and optional polypeptide B according to claim 1, wherein the polypeptide B comprises the amino acid sequence shown in SEQ ID NO: 103 or a functional variant thereof, and the functional variant has xanthine amide hydrolase activity.

19. A method for preventing, intervening and/or treating gout, comprising providing an individual in need the polypeptide A according to claim 1.

20. The nucleic acid molecule of claim 9, wherein polypeptide B has a catalytic site B defined as follows in spatial conformation: the catalytic site B comprises amino acid residues close to each other in spatial conformation, referring to the amino acid residues H59, H61, K151, H186, H242 and D316 of SEQ ID NO:103.

21. The nucleic acid molecule, the pharmaceutical composition or health food, the usage or the method according to claim 20, wherein the catalytic site B further comprises a divalent metal ion, optionally two divalent metals ions (e.g. Zn²⁺ and/or Mn²⁺).

22. The nucleic acid molecule according to claim 9, wherein the polypeptide B further comprises a binding site B having the following definition in a spatial conformation: the binding site B includes amino acid residues I288, A289, P338 and G339 of SEQ ID NO: 103 that are close to each other in spatial conformation.

23. The nucleic acid molecule, the pharmaceutical composition or health food, the usage or the method according to claim 22, wherein the distance between the catalytic site B and the binding site B is not more than 5 angstroms.

24. The nucleic acid molecule according to claim 9, wherein the functional variant with xanthine amide hydrolase activity is a natural amino acid sequence shown in SEQ ID NO: 103 isoenzyme-Preferably, the natural isoenzyme is from: Bacillus firmus, compost metagenome, Clostridium purinilyticum, Thermoflavimicrobium sp., Marininema halotolerans, Clostridiaceae bacterium, Bacillus sp., Paenibacillus sp., Thermoflavimicrobium dichotomicum, Fictibacillus enclensis, Marininema mesophilum, Paenibacillus typhae, Tissierella praeacuta, Bradyrhizobium japonicum, Acidaminobacter hydrogenoformans, Caloranaerobacter sp., Tissierella sp., Paenibacillus donghaensis, Gottschalkia acidurici, Clostridium acidurici, Bacillus fortis, Bacillus oceanisediminis, Virgibacillus profundi, Anaeromicrobium sediminis, Thermosyntropha lipolytica, Alkaliphilus peptidifermentans, Aneurinibacillus migulanus, Bacillus bacterium, Marinasporobacter balticus, Bacillus terrae, Tindallia calforniensis, Romboutsia lituseburensis, Paraclostridium bifermentans, Bacillus praedii, Carbydothermus islandicus, Caloramator australicus, Paraclostridium bifermentans, Tepidimicrobium xylanilyticum, Bacillus notoginsengisoli, [Clostridium] ultunense Esp, Bacillus freudenreichii, Caloramator fervidus, Soehngenia saccharinilytica, Thermotalea metallivorans, Ornithinibacillus halophilus, Fictibacillus sp., Bacillus mesonae, Tindallia magadiensis, Paenibacillus borealis, Paraclostridium benzoelyticum, Bacillus solani, Thermohalobacter berrensis, Acinetobacter sp., Maledivibacter halophilus, Tissierella creatinine, Natronincola peptidivorans, Anaerovirgula multiredigens, Carbydothermus hydrogenoformans, Sporanaerobacter acetigenes, Proteiniborus sp., Virgibacillus indicus, Andreesenia angusta, Paludifilum halophilum, Proteiniborus ethanoligenes, Alkaliphilus metalliredigens, Fictibacillus solisalsi, Sporosarcina globispora, Alkaliphilus sp., Alkaliphilus oremlandii, Caloramator mitchellensis, Clostridium cylindrosporum, Ammoniphilus sp., Carbydothermus pertinax, Soehngenia sp. and Natribacillus halophilus.

25. The nucleic acid molecule according to claim 9, wherein the functional variant with xanthine amide hydrolase activity is the amino acid sequence shown in SEQ ID NO: 103 or its natural isoform and is produced by the insertion, substitution and/or deletion of one or more amino acids based on the enzyme. Optionally, the insertion, substitution and/or deletion does not occur at the catalytic site B and/or the binding site B.
